(19) United States
(12) Patent Application Publication
(10) Pub. No.: US 2025/0042895 A1
(43) Pub. Date: Feb. 6, 2025

(54) INHIBITORS OF ULK1 AND METHODS OF USE

(71) Applicant: H. LEE MOFFITT CANCER CENTER AND RESEARCH INSTITUTE, INC., Tampa, FL (US)

(72) Inventors: Andrii MONASTYRSKYI, Lutz, FL (US); Derek DUCKETT, Land O Lakes, FL (US); Alisa MOROZOVA, Tampa, FL (US); Sean CHIN CHAN, Tampa, FL (US)

(21) Appl. No.: 18/697,835

(22) PCT Filed: Oct. 6, 2022
(86) PCT No.: PCT/US2022/045876

§ 371 Date: Apr. 2, 2024

Related U.S. Application Data
(60) Provisional application No. 63/252,739, filed on Oct. 6, 2021.

(51) Int. Cl. C07D 471/04; A61K 31/437; A61K 31/444; A61K 31/4545; A61K 31/496; A61K 31/5377; A61K 31/55; C07D 519/00

(52) U.S. Cl. CPC C07D 471/04; A61K 31/437; A61K 31/444; A61K 31/4545; A61K 31/496; A61K 31/5377; A61K 31/55; C07D 519/00

(57) ABSTRACT

The present disclosure provides compounds which find use as ULK1 inhibitors and their use in the treatment of medical disorders, in particular disorders associated with abnormal cellular proliferation such as cancers.

### CROSS-REFERENCE TO RELATED APPLICATIONS

This application claims the benefit of priority to 63/252,739, filed Oct. 6, 2021, the disclosure of which is incorporated herein by reference in its entirety.

### TECHNICAL FIELD

This disclosure relates to compounds for the treatment of medical disorders, and more particularly to ULK1 inhibitors which may be used in the treatment of proliferative disorders such as cancer.

### BACKGROUND

Autophagy is an ancient, cannibalistic (literally “self-eating”) pathway in which building blocks of the cell are recycled via the lysosome, both as a homeostatic mechanism and as a survival strategy during nutrient limitation or times of stress.^{[1-3]} The pathway is initiated by the formation of isolation membranes (phagophores), which are derived from the outer membrane of the mitochondria and/or the ER. These then fuse to form double-membrane (neutral pH) vesicles coined autophagosomes that engulf their cargo, which typically includes long-lived proteins, bulk cytoplasmic material and aged or damaged organelles (e.g., mitochondria). Autophagosomes then fuse with the lysosome, which degrades the delivered cargo to recoup essential building blocks and ATP necessary for cell survival.^{[4]} Aberrant regulation of autophagy is associated with a variety of human conditions, including cancer.^{[5, 6]} The observed role of the autophagy pathway in cancer is complex, where it can act as a tumor suppressor to prevent initiation of cancer^{[7]} or as an essential mediator of tumor progression.^{[8, 9]} Flux through the autophagy pathway is markedly elevated in tumors driven by oncoproteins such as mutant KRAS, and in tumor cells exposed to noxious cues, for example to hypoxia, reactive oxygen species, chemotherapeutic agents or radiation. Accordingly, the induction of autophagy can drive tumor progression and contributes to chemotherapy resistance associated with relapsed disease. Further, it has been shown that inhibiting autophagy augments the efficacy of both conventional and targeted anti-cancer agents, including otherwise resistant malignancies.^{[10-12]}

Notably, while several activators and inhibitors of autophagy are known (i.e. chloroquine and hydroxychloroquine), these all are non-specific, and none target the enzymes that execute the pathway. A key regulator of the autophagy pathway and tractable target is the serine/threonine kinase, which is coined ATG1 in yeast. There are three homologs of ATG1 in vertebrates, ULK1, ULK2, and ULK3 (uncoordinated family member [Unc]-51-like kinases 1-3), yet only ULK1 is widely expressed.^{[13]} The autophagy pathway is suppressed by PI3K-AKT-mTOR and is activated by AMP kinase (AMPK), where both ULK1 and ATG13 are substrates of mTOR and AMPK.^{[14-18]}

There is a clear need for new inhibitors of ULK1 which may be used in the treatment of proliferative disease and conditions, including cancers and other autophagy-mediated diseases. The compositions and methods disclosed herein address these and other needs.

### SUMMARY

In accordance with the purposes of the disclosed materials and methods, as embodied and broadly described herein, the disclosed subject matter, in one aspect, relates to compounds, compositions, and methods of making and use compounds and compositions.

In particular, the present disclosure provides a compound of Formula I or Formula II:

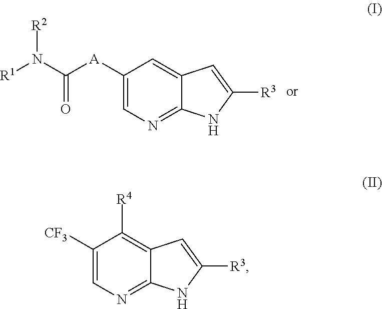

or a pharmaceutically acceptable salt, solvate, prodrug, or derivative thereof, wherein all variables are as further defined herein. The present disclosure also provides pharmaceutical compositions comprising a compound of Formula I or Formula II, or a pharmaceutically acceptable salt, solvate, prodrug, or derivative thereof, and a pharmaceutically acceptable carrier.

The present disclosure further provides methods of treating a proliferative disorder, for example a cancer, in a subject in need thereof comprising administering to the subject a therapeutically effective amount of a compound of Formula I or Formula II, or a pharmaceutically acceptable salt, solvate, prodrug, or derivative thereof, optionally as a pharmaceutical composition. In some embodiments, the proliferative disorder comprises a cancer selected from lung cancer, pancreatic cancer, colorectal cancer, leukemias, lymphoma, melanoma, multiple myeloma, Ewing's sarcoma, osteosarcoma, gastric adenocarcinoma, cervical adenocarcinoma, and head and neck squamous cell carcinoma.

The details of one or more embodiments of the disclosure are set forth in the description below. Other features, objects, and advantages of the disclosure will be apparent from the description and the claims.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a compound”, “a composition”, or “a tumor”, includes, but is not limited to, two or more such compounds, compositions, or tumors, and the like.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose.

The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound or composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a proliferative disorder. The effect can be therapeutic in terms of a partial or complete cure of a disease, condition, symptom or adverse effect attributed to the disease, disorder, or condition. The term “treatment” as used herein can include any treatment of a disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

Chemical Definitions Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R—) or (S—) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R—) form is equivalent, for compounds that undergo epimerization in vivo, to administration of the compound in its (S—) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine.

Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙ alkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. “Cycloalkenyl” is a mono- or multi-cyclic hydrocarbon ring system having one or more double bonds. In one embodiment, the cycloalkyl or cycloalkenyl group is optionally substituted as described herein. “Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Halo” or “halogen” indicates, independently, any of fluoro, chloro, bromo or iodo.

“Haloalkyl” is an alkyl group as defined above substituted with at least one halo substituent.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl.

Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl.

Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 3, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfone, ethane disulfonic, oxalic, isethionic, HOOC—(CH₂)₁₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### Compounds

The present disclosure provides compounds which find use as inhibitors of Unc-51 Like Autophagy Activity Kinase 1 (ULK1) and which are useful in the treatment of disorders associated with abnormal cell proliferation and/or mediated by autophagy.

In one aspect, a compound is provided of Formula I:

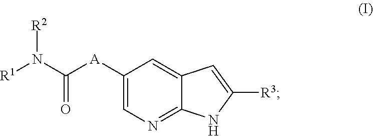

or a pharmaceutically acceptable salt, solvate, prodrug, or derivative thereof;
wherein:
A is selected from: 6- to 10-membered monocyclic or bicyclic aryl; and 5- to 10-membered monocyclic or bicyclic heteroaryl; wherein A is optionally substituted with one or more groups (for example, 1, 2, 3, or 4 groups) selected from X;
R¹ is selected from C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
R² is selected from hydrogen, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
or R¹ and R² are brought together with the nitrogen atom to which they are attached to form a 3- to 8-membered monocyclic or bicyclic heterocycle or a 5- to 10-membered monocyclic or bicyclic heteroaryl, each of which is optionally substituted with one or more groups (for example, 1, 2, 3, or 4 groups) selected from X;
R³ is selected from: C₃-C₈ monocyclic or bicyclic cycloalkyl; C₃-C₆ cycloalkenyl; 3- to 8-membered monocyclic or bicyclic heterocycle; 6- to 10-membered monocyclic or bicyclic aryl; and 5- to 10-membered monocyclic or bicyclic heteroaryl; wherein R³ is optionally substituted with one or more groups (for example, 1, 2, 3, or 4 groups) selected from X; or
R³ is —B—C;
B is selected from: C₃-C₈ monocyclic or bicyclic cycloalkyl; C₃-C₆ cycloalkenyl; 3- to 8-membered monocyclic or bicyclic heterocycle; wherein B is optionally substituted with one or more groups (for example, 1, 2, 3, or 4 groups) selected from X; C is 3- to 8-membered monocyclic or bicyclic heterocycle which is optionally substituted with one or more groups (for example, 1, 2, 3, or 4 groups) selected from X;
X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which is optionally substituted with one or more groups (for example, 1, 2, 3, or 4 groups) selected from Y;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some embodiments of Formula I, A is 6- to 10-membered monocyclic or bicyclic aryl optionally substituted with 1, 2, 3, or 4 groups selected from X. In some embodiments of Formula I, A is 5- to 10-membered monocyclic or bicyclic heteroaryl optionally substituted with 1, 2, 3, or 4 groups selected from X.

In some embodiments of Formula I, A is selected from:

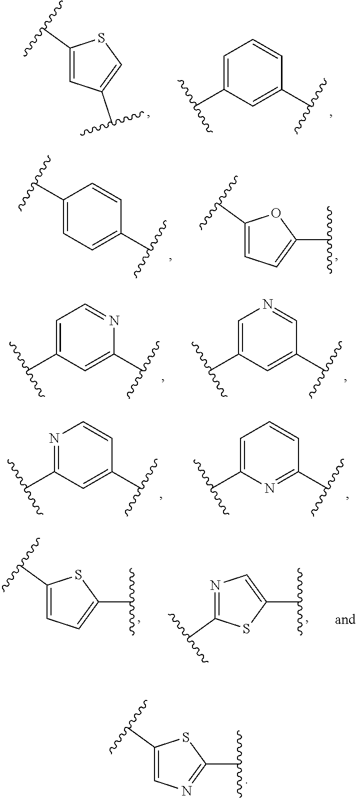

In some embodiments of Formula I, R³ is C₃-C₈ monocyclic or bicyclic cycloalkyl optionally substituted with 1, 2, 3, or 4 groups selected from X. In some embodiments of Formula I, R³ is C₃-C₆ cycloalkenyl optionally substituted with 1, 2, 3, or 4 groups selected from X. In some embodiments of Formula I, R³ is 3- to 8-membered monocyclic or bicyclic heterocycle optionally substituted with 1, 2, 3, or 4 groups selected from X. In some embodiments of Formula I, R³ is 6- to 10-membered monocyclic or bicyclic aryl optionally substituted with 1, 2, 3, or 4 groups selected from X.

In some embodiments of Formula I, R³ is selected from:

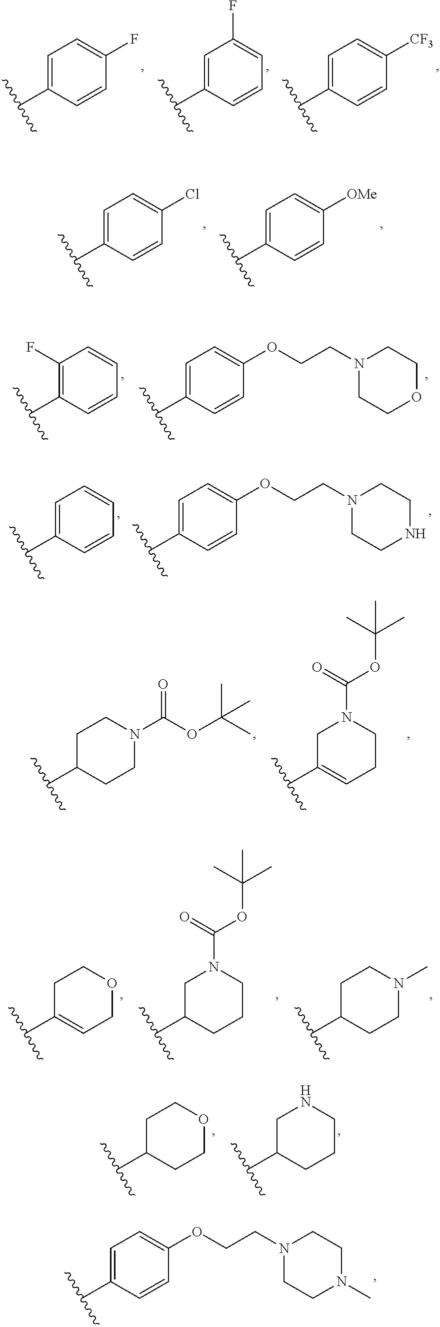

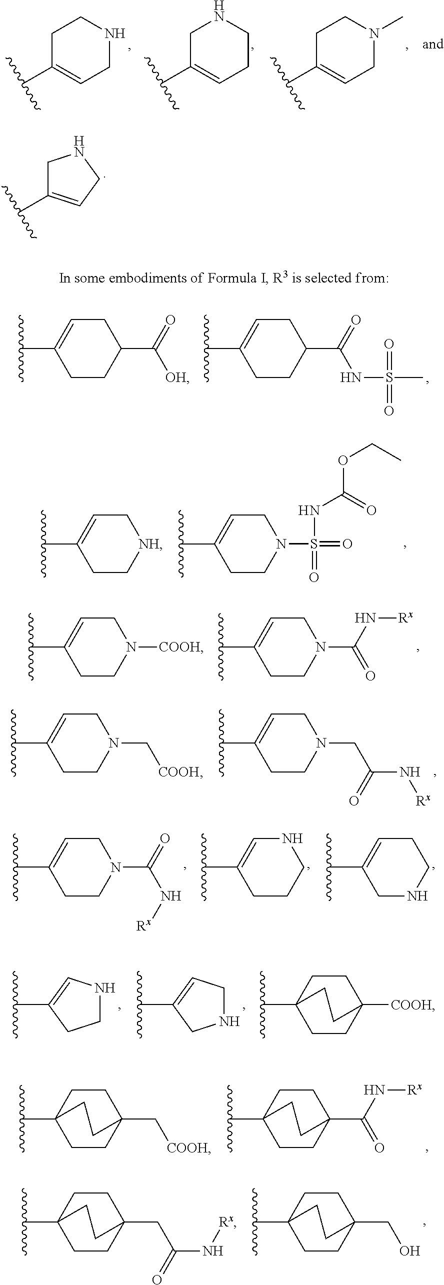

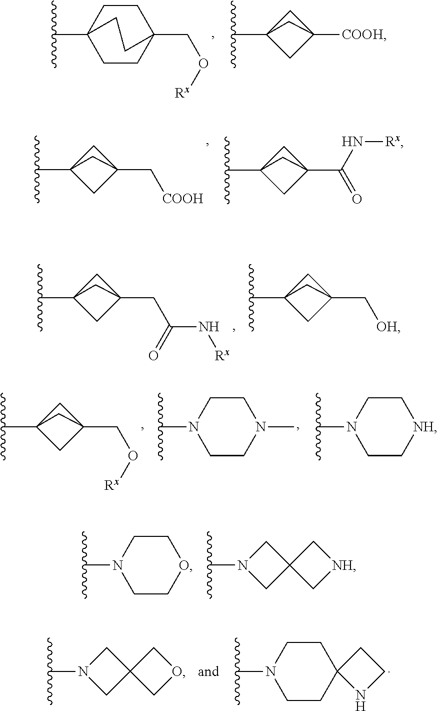

In some embodiments of Formula I, R³ is —B—C.

In some embodiments of Formula I, B is C₃-C₈ monocyclic or bicyclic cycloalkyl optionally substituted with 1, 2, 3, or 4 groups selected from X. In some embodiments of Formula I, B is C₃-C₆ cycloalkenyl optionally substituted with 1, 2, 3, or 4 groups selected from X. In some embodiments of Formula I, B is 3- to 8-membered monocyclic or bicyclic heterocycle optionally substituted with 1, 2, 3, or 4 groups selected from X.

In some embodiments of Formulas I, C is 3- to 8-membered monocyclic or bicyclic heterocycle optionally substituted with 1, 2, 3, or 4 groups selected from X.

In some embodiments of Formula I, R³ is selected from:

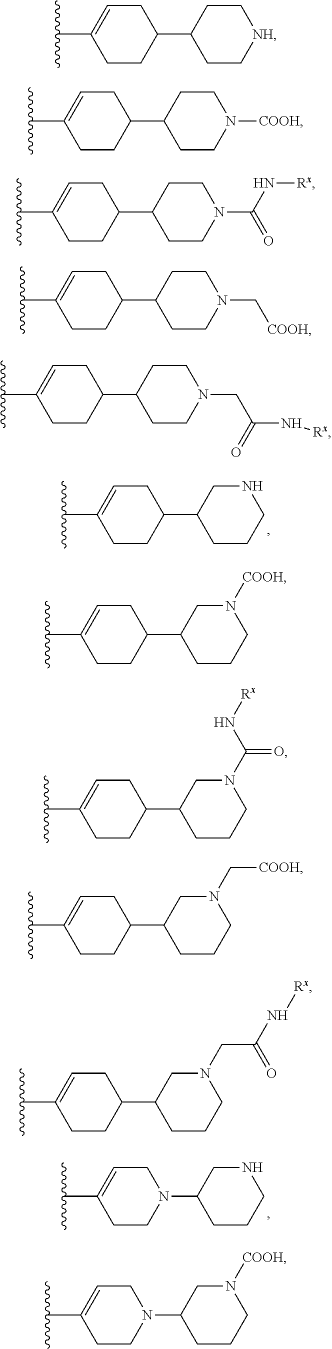

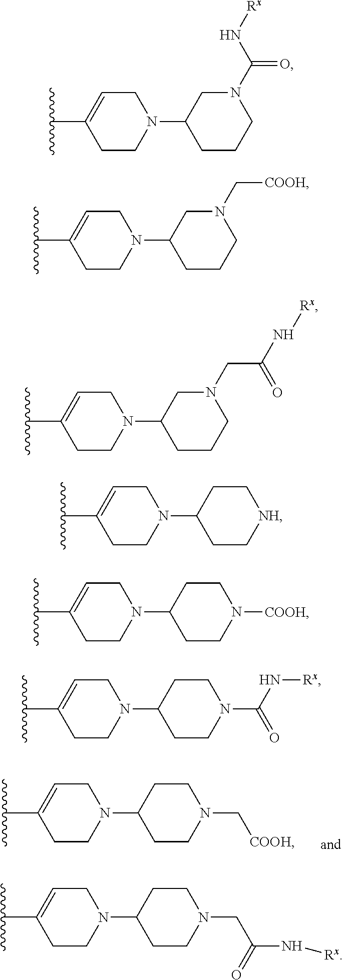

In some embodiments of Formula I, R² is hydrogen.

In some embodiments of Formula I, R¹ is selected from:

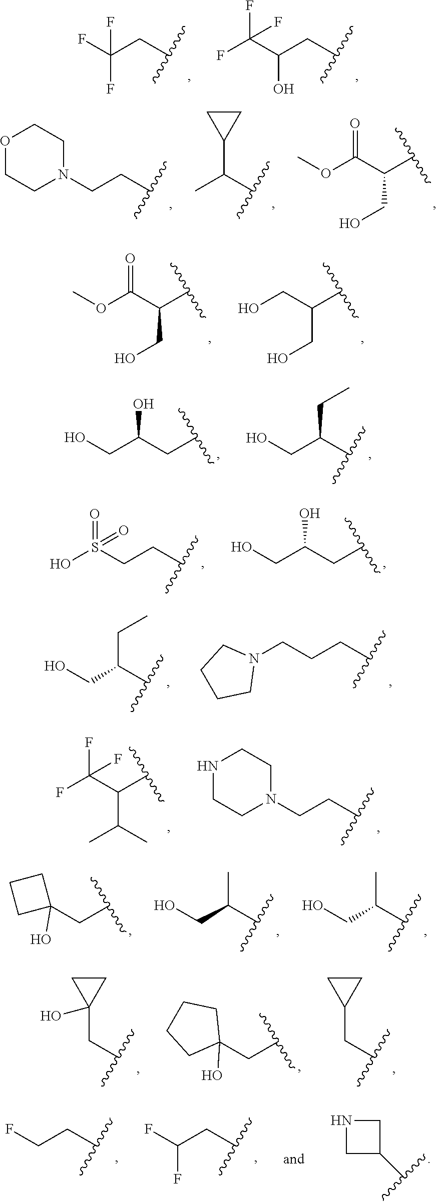

In some embodiments of Formula I, R¹ and R² are brought together with the nitrogen to which they are attached to form a group selected from:

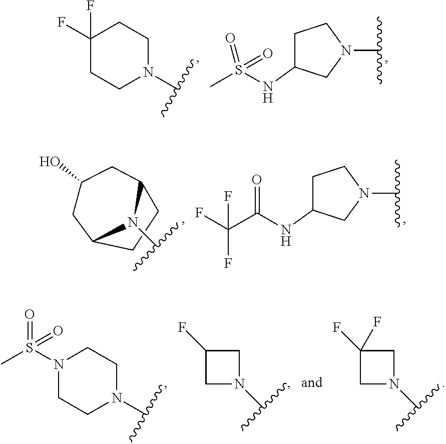

Representative compounds of Formula I include, but are not limited to, the compounds provided in Table 1 below, or pharmaceutically acceptable salts, prodrugs, or derivatives thereof.

TABLE 1
Exemplary Compounds of Formula I

| Structure | | |
|---|---|---|
| Cmpd # | Name | MW |
| 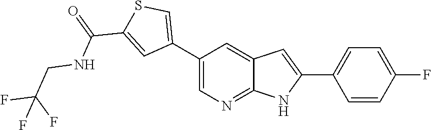 [SMILES: O=C(NCC(F)(F)F)c1cc(-c2cnc3[nH]c(-c4ccc(F)cc4)cc3c2)cs1] | 4-(2-(4-fluorophenyl)-1H-pyrrolo- [2,3-b]pyridin-5-yl)-N-(2,2,2- trifluoroethyl)thiophene-2- carboxamide | 419.4 |
| 1 | | |
| 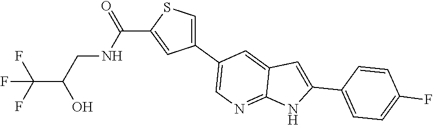 [SMILES: O=C(NCC(O)C(F)(F)F)c1cc(-c2cnc3[nH]c(-c4ccc(F)cc4)cc3c2)cs1] | 4-(2-(4-fluorophenyl)-1H-pyrrolo- [2,3-b]pyridin-5-yl)-N-(3,3,3- trifluoro-2- hydroxypropyl)thiophene-2- carboxamide | 449.4 |
| 2 | | |
| 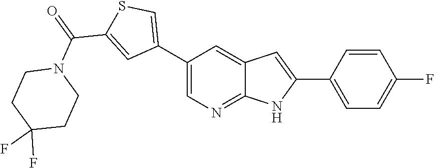 [SMILES: O=C(c1cc(-c2cnc3[nH]c(-c4ccc(F)cc4)cc3c2)cs1)N1CCC(F)(F)CC1] | (4,4-difluoropiperidin-1-yl)(4-(2- (4-fluorophenyl)-1H-pyrrolo[2,3- b]pyridin-5-yl)thiophen-2- yl)methanone | 441.5 |
| 3 | | |
| 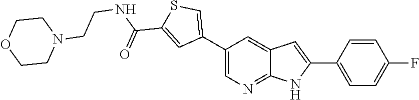 [SMILES: O=C(NCCN1CCOCC1)c1cc(-c2cnc3[nH]c(-c4ccc(F)cc4)cc3c2)cs1] | 4-(2-(4-fluorophenyl)-1H-pyrrolo- [2,3-b]pyridin-5-yl)-N-(2- morpholinoethyl)thiophene-2- carboxamide | 450.5 |
| 4 | | |
| 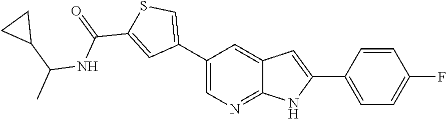 [SMILES: CC(NC(=O)c1cc(-c2cnc3[nH]c(-c4ccc(F)cc4)cc3c2)cs1)C1CC1] | N-(1-cyclopropylethyl)-4-(2-(4- fluorophenyl)-1H-pyrrolo[2,3-b]- pyridin-5-yl)thiophene-2- carboxamide | 405.5 |
| 5 | | |
| 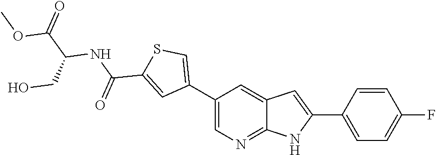 [SMILES: COC(=O)[C@@H](CO)NC(=O)c1cc(-c2cnc3[nH]c(-c4ccc(F)cc4)cc3c2)cs1] | methyl (4-(2-(4-fluorophenyl)-1H- pyrrolo[2,3-b]pyridin-5-yl)- thiophene-2-carbonyl)-D-serinate | 439.5 |
| 6 | | |
| 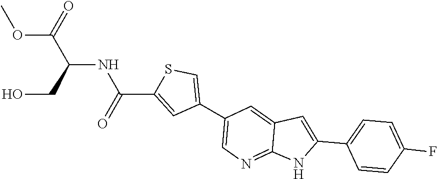 [SMILES: COC(=O)[C@H](CO)NC(=O)c1cc(-c2cnc3[nH]c(-c4ccc(F)cc4)cc3c2)cs1] | methyl (4-(2-(4-fluorophenyl)-1H- pyrrolo[2,3-b]pyridin-5-yl)- thiophene-2-carbonyl)-L-serinate | 439.5 |
| 7 | | |
| 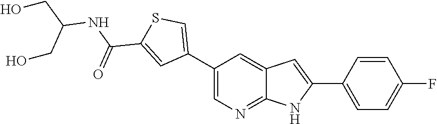 [SMILES: O=C(NC(CO)CO)c1cc(-c2cnc3[nH]c(-c4ccc(F)cc4)cc3c2)cs1] | N-(1,3-dihydroxypropan-2-yl)-4-(2- (4-fluorophenyl)-1H-pyrrolo[2,3- b]pyridin-5-yl)thiophene-2- carboxamide | 411.5 |
| 8 | | |
| 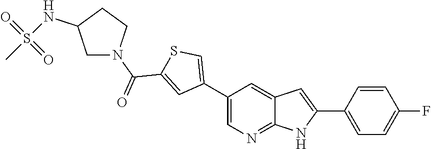 [SMILES: CS(=O)(=O)NC1CCN(C(=O)c2cc(-c3cnc4[nH]c(-c5ccc(F)cc5)cc4c3)cs2)C1] | N-(1-(4-(2-(4-fluorophenyl)-1H- pyrrolo[2,3-b]pyridin-5-yl)- thiophene-2-carbonyl)pyrrolidin-3- yl)methanesulfonamide | 484.6 |
| 9 | | |
| 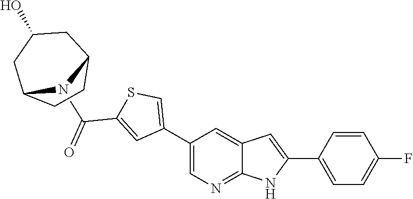 [SMILES: O=C(c1cc(-c2cnc3[nH]c(-c4ccc(F)cc4)cc3c2)cs1)N1[C@@H]2CC[C@H]1C[C@@H](O)C2] | (4-(2-(4-fluorophenyl)-1H-pyrrolo- [2,3-b]pyridin-5-yl)thiophen-2-yl)- ((1R,3r,5S)-3-hydroxy-8- azabicyclo[3.2.1]octan-8- yl)methanone | 447.5 |
| 10 | | |
| 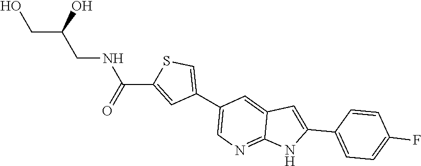 [SMILES: O=C(NC[C@H](O)CO)c1cc(-c2cnc3[nH]c(-c4ccc(F)cc4)cc3c2)cs1] | (S)-N-(2,3-dihydroxypropyl)-4-(2- (4-fluorophenyl)-1H-pyrrolo[2,3- b]pyridin-5-yl)thiophene-2- carboxamide | 411.5 |
| 11 | | |
| 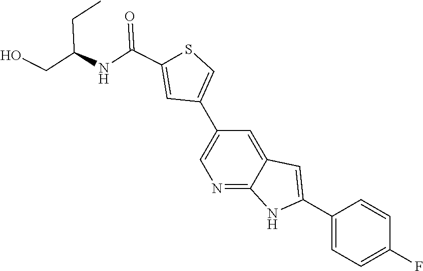 [SMILES: CC[C@H](CO)NC(=O)c1cc(-c2cnc3[nH]c(-c4ccc(F)cc4)cc3c2)cs1] | (R)-4-(2-(4-fluorophenyl)-1H- pyrrolo[2,3-b]pyridin-5-yl)-N-(1- hydroxybutan-2-yl)thiophene-2- carboxamide | 409.5 |
| 12 | | |
| 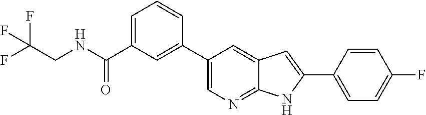 [SMILES: O=C(NCC(F)(F)F)c1cccc(-c2cnc3[nH]c(-c4ccc(F)cc4)cc3c2)c1] | 3-(2-(4-fluorophenyl)-1H-pyrrolo- [2,3-b]pyridin-5-yl)-N-(2,2,2- trifluoroethyl)benzamide | 413.4 |
| 13 | | |
| 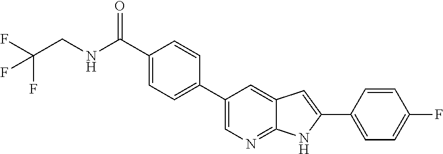 [SMILES: O=C(NCC(F)(F)F)c1ccc(-c2cnc3[nH]c(-c4ccc(F)cc4)cc3c2)cc1] | 4-(2-(4-fluorophenyl)-1H-pyrrolo- [2,3-b]pyridin-5-yl)-N-(2,2,2- trifluoroethyl)benzamide | 413.4 |
| 14 | | |
| 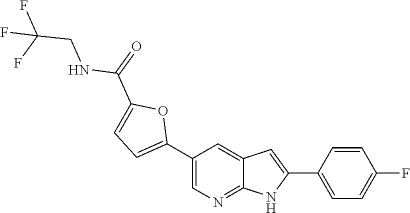 [SMILES: O=C(NCC(F)(F)F)c1ccc(-c2cnc3[nH]c(-c4ccc(F)cc4)cc3c2)o1] | 5-(2-(4-fluorophenyl)-1H-pyrrolo- [2,3-b]pyridin-5-yl)-N-(2,2,2- trifluoroethyl)furan-2-carboxamide | 403.3 |
| 15 | | |
| 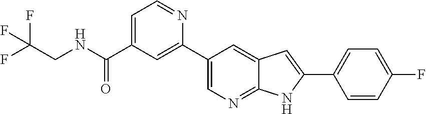 [SMILES: O=C(NCC(F)(F)F)c1ccnc(-c2cnc3[nH]c(-c4ccc(F)cc4)cc3c2)c1] | 2-(2-(4-fluorophenyl)-1H-pyrrolo- [2,3-b]pyridin-5-yl)-N-(2,2,2- trifluoroethyl)isonicotinamide | 414.4 |
| 16 | | |
| 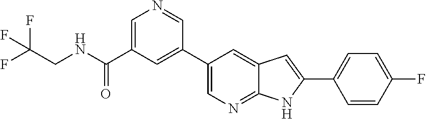 [SMILES: O=C(NCC(F)(F)F)c1cncc(-c2cnc3[nH]c(-c4ccc(F)cc4)cc3c2)c1] | 5-(2-(4-fluorophenyl)-1H-pyrrolo- [2,3-b]pyridin-5-yl)-N-(2,2,2- trifluoroethyl)nicotinamide | 414.4 |
| 17 | | |
| 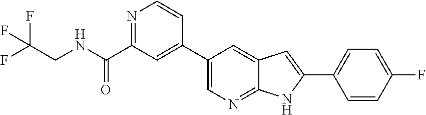 [SMILES: O=C(NCC(F)(F)F)c1cc(-c2cnc3[nH]c(-c4ccc(F)cc4)cc3c2)ccn1] | 4-(2-(4-fluorophenyl)-1H-pyrrolo- [2,3-b]pyridin-5-yl)-N-(2,2,2- trifluoroethyl)picolinamide | 414.4 |
| 18 | | |
| 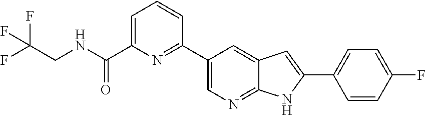 [SMILES: O=C(NCC(F)(F)F)c1cccc(-c2cnc3[nH]c(-c4ccc(F)cc4)cc3c2)n1] | 6-(2-(4-fluorophenyl)-1H-pyrrolo- [2,3-b]pyridin-5-yl)-N-(2,2,2- trifluoroethyl)picolinamide | 414.4 |
| 19 | | |
| 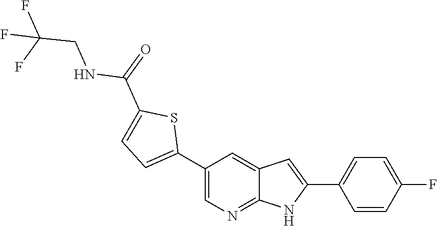 [SMILES: O=C(NCC(F)(F)F)c1ccc(-c2cnc3[nH]c(-c4ccc(F)cc4)cc3c2)s1] | 5-(2-(4-fluorophenyl)-1H-pyrrolo- [2,3-b]pyridin-5-yl)-N-(2,2,2- trifluoroethyl)thiophene-2- carboxamide | 419.4 |
| 20 | | |
| 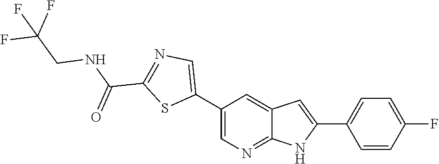 [SMILES: O=C(NCC(F)(F)F)c1ncc(-c2cnc3[nH]c(-c4ccc(F)cc4)cc3c2)s1] | 5-(2-(4-fluorophenyl)-1H-pyrrolo- [2,3-b]pyridin-5-yl)-N-(2,2,2- trifluoroethyl)thiazole-2- carboxamide | 420.4 |
| 21 | | |
| 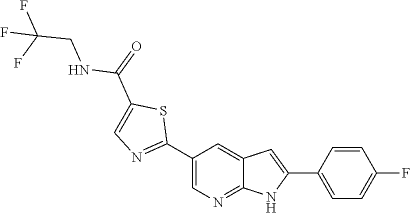 [SMILES: O=C(NCC(F)(F)F)c1cnc(-c2cnc3[nH]c(-c4ccc(F)cc4)cc3c2)s1] | 2-(2-(4-fluorophenyl)-1H-pyrrolo- [2,3-b]pyridin-5-yl)-N-(2,2,2- trifluoroethyl)thiazole-5- carboxamide | 420.4 |
| 22 | | |
| 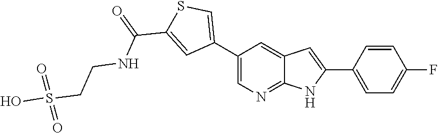 [SMILES: O=C(NCCS(=O)(=O)O)c1cc(-c2cnc3[nH]c(-c4ccc(F)cc4)cc3c2)cs1] | 2-(4-(2-(4-fluorophenyl)-1H- pyrrolo[2,3-b]pyridin-5- yl)thiophene-2- carboxamido)ethane-1-sulfonic acid | 445.5 |
| 23 | | |
| 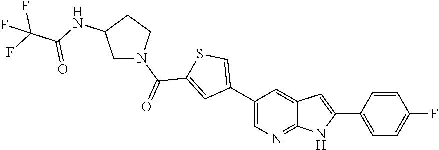 [SMILES: O=C(c1cc(-c2cnc3[nH]c(-c4ccc(F)cc4)cc3c2)cs1)N1CCC(NC(=O)C(F)(F)F)C1] | 2,2,2-trifluoro-N-(1-(4-(2-(4- fluorophenyl)-1H-pyrrolo[2,3-b]- pyridin-5-yl)thiophene-2-carbonyl)- pyrrolidin-3-yl)acetamide | 502.5 |
| 24 | | |
| 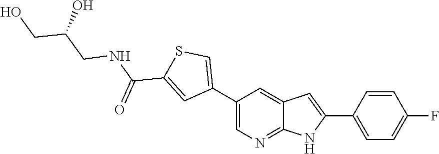 [SMILES: O=C(NC[C@@H](O)CO)c1cc(-c2cnc3[nH]c(-c4ccc(F)cc4)cc3c2)cs1] | (R)-N-(2,3-dihydroxypropyl)-4-(2- (4-fluorophenyl)-1H-pyrrolo[2,3- b]pyridin-5-yl)thiophene-2- carboxamide | 411.5 |
| 25 | | |
| 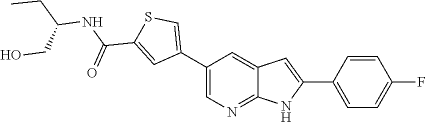 [SMILES: CC[C@@H](CO)NC(=O)c1cc(-c2cnc3[nH]c(-c4ccc(F)cc4)cc3c2)cs1] | (S)-4-(2-(4-fluorophenyl)-1H- pyrrolo[2,3-b]pyridin-5-yl)-N-(1- hydroxybutan-2-yl)thiophene-2- carboxamide | 409.5 |
| 26 | | |
| 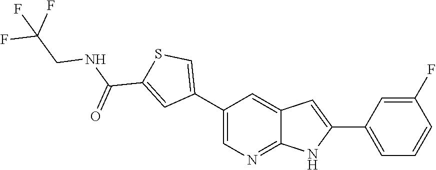 [SMILES: O=C(NCC(F)(F)F)c1cc(-c2cnc3[nH]c(-c4cccc(F)c4)cc3c2)cs1] | 4-(2-(3-fluorophenyl)-1H-pyrrolo- [2,3-b]pyridin-5-yl)-N-(2,2,2- trifluoroethyl)thiophene-2- carboxamide | 419.4 |
| 27 | | |
| 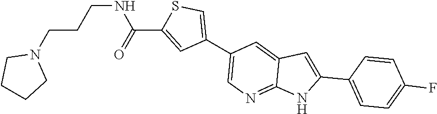 [SMILES: O=C(NCCCN1CCCC1)c1cc(-c2cnc3[nH]c(-c4ccc(F)cc4)cc3c2)cs1] | 4-(2-(4-fluorophenyl)-1H-pyrrolo- [2,3-b]pyridin-5-yl)-N-(3- (pyrrolidin-1-yl)propyl)thiophene- 2-carboxamide | 448.6 |
| 28 | | |
| 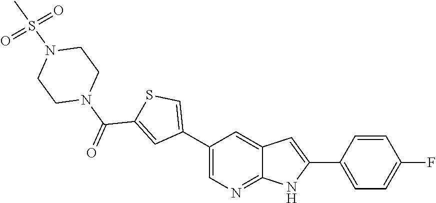 [SMILES: CS(=O)(=O)N1CCN(C(=O)c2cc(-c3cnc4[nH]c(-c5ccc(F)cc5)cc4c3)cs2)CC1] | (4-(2-(4-fluorophenyl)-1H-pyrrolo- [2,3-b]pyridin-5-yl)thiophen-2- yl)(4-(methylsulfonyl)piperazin-1- yl)methanone | 484.6 |
| 29 | | |
| 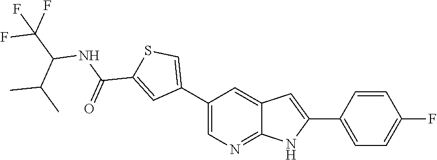 [SMILES: CC(C)C(NC(=O)c1cc(-c2cnc3[nH]c(-c4ccc(F)cc4)cc3c2)cs1)C(F)(F)F] | 4-(2-(4-fluorophenyl)-1H- pyrrolo[2,3-b]pyridin-5-yl)-N- (1,1,1-trifluoro-3-methylbutan-2- yl)thiophene-2-carboxamide | 461.5 |
| 30 | | |
| 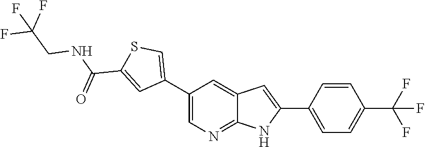 [SMILES: O=C(NCC(F)(F)F)c1cc(-c2cnc3[nH]c(-c4ccc(C(F)(F)F)cc4)cc3c2)cs1] | N-(2,2,2-trifluoroethyl)-4-(2-(4- (trifluoromethyl)phenyl)-1H- pyrrolo[2,3-b]pyridin-5- yl)thiophene-2-carboxamide | 469.4 |
| 31 | | |
| 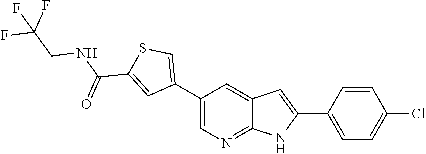 [SMILES: O=C(NCC(F)(F)F)c1cc(-c2cnc3[nH]c(-c4ccc(Cl)cc4)cc3c2)cs1] | 4-(2-(4-chlorophenyl)-1H-pyrrolo- [2,3-b]pyridin-5-yl)-N-(2,2,2- trifluoroethyl)thiophene-2- carboxamide | 435.8 |
| 32 | | |
| 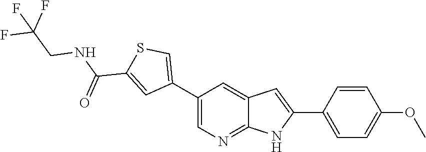 [SMILES: COc1ccc(-c2cc3cc(-c4csc(C(=O)NCC(F)(F)F)c4)cnc3[nH]2)cc1] | 4-(2-(4-methoxyphenyl)-1H- pyrrolo[2,3-b]pyridin-5-yl)-N- (2,2,2-trifluoroethyl)thiophene-2- carboxamide | 431.4 |
| 33 | | |
| 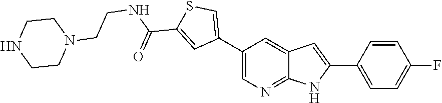 [SMILES: O=C(NCCN1CCNCC1)c1cc(-c2cnc3[nH]c(-c4ccc(F)cc4)cc3c2)cs1] | 4-(2-(4-fluorophenyl)-1H-pyrrolo- [2,3-b]pyridin-5-yl)-N-(2- (piperazin-1-yl)ethyl)thiophene-2- carboxamide | 449.5 |
| 34 | | |
| 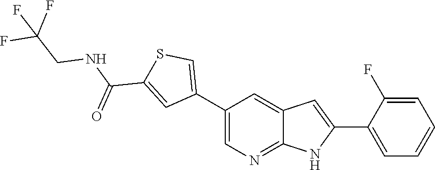 [SMILES: O=C(NCC(F)(F)F)c1cc(-c2cnc3[nH]c(-c4ccccc4F)cc3c2)cs1] | 4-(2-(2-fluorophenyl)-1H-pyrrolo- [2,3-b]pyridin-5-yl)-N-(2,2,2- trifluoroethyl)thiophene-2- carboxamide | 419.4 |
| 35 | | |
| 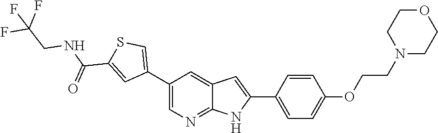 [SMILES: O=C(NCC(F)(F)F)c1cc(-c2cnc3[nH]c(-c4ccc(OCCN5CCOCC5)cc4)cc3c2)cs1] | 4-(2-(4-(2-morpholinoethoxy)- phenyl)-1H-pyrrolo[2,3-b]pyridin- 5-yl)-N-(2,2,2-trifluoroethyl)- thiophene-2-carboxamide | 530.6 |
| 36 | | |
| 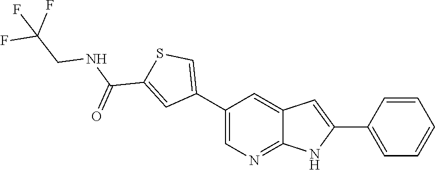 [SMILES: O=C(NCC(F)(F)F)c1cc(-c2cnc3[nH]c(-c4ccccc4)cc3c2)cs1] | 4-(2-phenyl-1H-pyrrolo[2,3-b]- pyridin-5-yl)-N-(2,2,2- trifluoroethyl)thiophene-2- carboxamide | 401.4 |
| 37 | | |
| 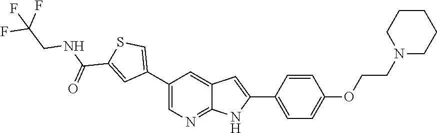 [SMILES: O=C(NCC(F)(F)F)c1cc(-c2cnc3[nH]c(-c4ccc(OCCN5CCCCC5)cc4)cc3c2)cs1] | 4-(2-(4-(2-(piperidin-1-yl)ethoxy)- phenyl)-1H-pyrrolo[2,3-b]pyridin- 5-yl)-N-(2,2,2-trifluoroethyl)- thiophene-2-carboxamide | 528.6 |
| 38 | | |
| 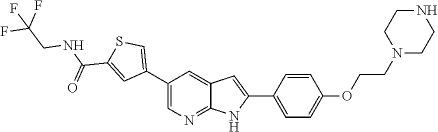 [SMILES: O=C(NCC(F)(F)F)c1cc(-c2cnc3[nH]c(-c4ccc(OCCN5CCNCC5)cc4)cc3c2)cs1] | 4-(2-(4-(2-(piperazin-1-yl)ethoxy)- phenyl)-1H-pyrrolo[2,3-b]pyridin- 5-yl)-N-(2,2,2-trifluoroethyl)- thiophene-2-carboxamide | 529.6 |
| 39 | | |
| 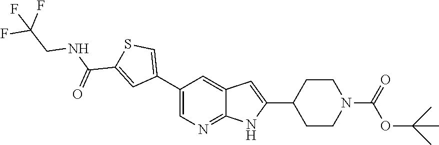 [SMILES: CC(C)(C)OC(=O)N1CCC(c2cc3cc(-c4csc(C(=O)NCC(F)(F)F)c4)cnc3[nH]2)CC1] | tert-butyl 4-(5-(5-((2,2,2-trifluoro- ethyl)carbamoyl)thiophen-3-yl)- 1H-pyrrolo[2,3-b]pyridin-2-yl)- piperidine-1-carboxylate | 508.6 |
| 40 | | |
| 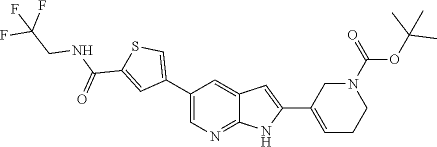 [SMILES: CC(C)(C)OC(=O)N1CCC=C(c2cc3cc(-c4csc(C(=O)NCC(F)(F)F)c4)cnc3[nH]2)C1] | tert-butyl 5-(5-(5-((2,2,2-trifluoro- ethyl)carbamoyl)thiophen-3-yl)- 1H-pyrrolo[2,3-b]pyridin-2-yl)-3,6- dihydropyridine-1(2H)-carboxylate | 506.5 |
| 41 | | |
| 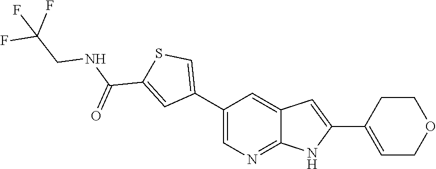 [SMILES: O=C(NCC(F)(F)F)c1cc(-c2cnc3[nH]c(C4=CCOCC4)cc3c2)cs1] | 4-(2-(3,6-dihydro-2H-pyran-4-yl)- 1H-pyrrolo[2,3-b]pyridin-5-yl)-N- (2,2,2-trifluoroethyl)thiophene-2- carboxamide | 407.4 |
| 42 | | |
| 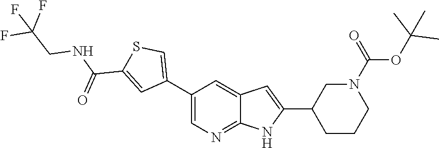 [SMILES: CC(C)(C)OC(=O)N1CCCC(c2cc3cc(-c4csc(C(=O)NCC(F)(F)F)c4)cnc3[nH]2)C1] | tert-butyl 3-(5-(5-((2,2,2-trifluoro- ethyl)carbamoyl)thiophen-3-yl)- 1H-pyrrolo[2,3-b]pyridin-2-yl)- piperidine-1-carboxylate | 508.6 |
| 43 | | |
| 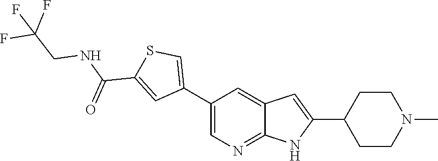 [SMILES: CN1CCC(c2cc3cc(-c4csc(C(=O)NCC(F)(F)F)c4)cnc3[nH]2)CC1] | 4-(2-(1-methylpiperidin-4-yl)-1H- pyrrolo[2,3-b]pyridin-5-yl)-N- (2,2,2-trifluoroethyl)thiophene-2- carboxamide | 422.5 |
| 44 | | |
| 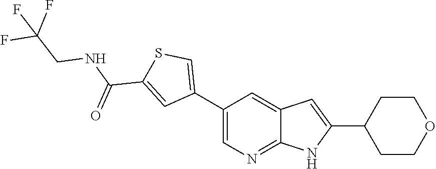 [SMILES: O=C(NCC(F)(F)F)c1cc(-c2cnc3[nH]c(C4CCOCC4)cc3c2)cs1] | 4-(2-(tetrahydro-2H-pyran-4-yl)- 1H-pyrrolo[2,3-b]pyridin-5-yl)-N- (2,2,2-trifluoroethyl)thiophene-2- carboxamide | 409.4 |
| 45 | | |
| 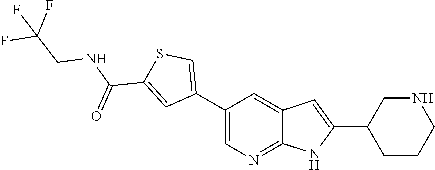 [SMILES: O=C(NCC(F)(F)F)c1cc(-c2cnc3[nH]c(C4CCCNC4)cc3c2)cs1] | 4-(2-(piperidin-3-yl)-1H- pyrrolo[2,3-b]pyridin-5-yl)-N- (2,2,2-trifluoroethyl)thiophene-2- carboxamide | 408.4 |
| 46 | | |
| 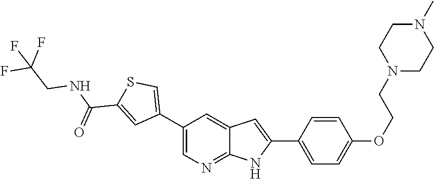 [SMILES: CN1CCN(CCOc2ccc(-c3cc4cc(-c5csc(C(=O)NCC(F)(F)F)c5)cnc4[nH]3)cc2)CC1] | 4-(2-(4-(2-(4-methylpiperazin-1- yl)ethoxy)phenyl)-1H-pyrrolo[2,3- b]pyridin-5-yl)-N-(2,2,2- trifluoroethyl)thiophene-2- carboxamide | 543.6 |
| 47 | | |
| 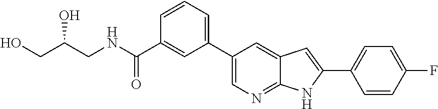 [SMILES: O=C(NC[C@@H](O)CO)c1cccc(-c2cnc3[nH]c(-c4ccc(F)cc4)cc3c2)c1] | (R)-N-(2,3-dihydroxypropyl)-3-(2- (4-fluorophenyl)-1H-pyrrolo[2,3- b]pyridin-5-yl)benzamide | 405.4 |
| 48 | | |
| 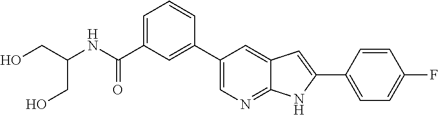 [SMILES: O=C(NC(CO)CO)c1cccc(-c2cnc3[nH]c(-c4ccc(F)cc4)cc3c2)c1] | N-(1,3-dihydroxypropan-2-yl)-3-(2- (4-fluorophenyl)-1H-pyrrolo[2,3- b]pyridin-5-yl)benzamide | 405.4 |
| 49 | | |
| 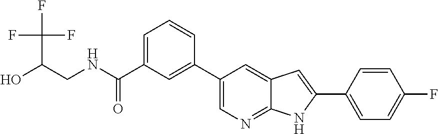 [SMILES: O=C(NCC(O)C(F)(F)F)c1cccc(-c2cnc3[nH]c(-c4ccc(F)cc4)cc3c2)c1] | 3-(2-(4-fluorophenyl)-1H-pyrrolo- [2,3-b]pyridin-5-yl)-N-(3,3,3-tri- fluoro-2-hydroxypropyl)benzamide | 443.4 |
| 50 | | |
| 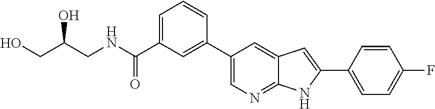 [SMILES: O=C(NC[C@H](O)CO)c1cccc(-c2cnc3[nH]c(-c4ccc(F)cc4)cc3c2)c1] | (S)-N-(2,3-dihydroxypropyl)-3-(2- (4-fluorophenyl)-1H-pyrrolo[2,3- b]pyridin-5-yl)benzamide | 405.4 |
| 51 | | |
| 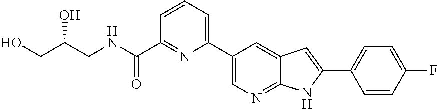 [SMILES: O=C(NC[C@@H](O)CO)c1cccc(-c2cnc3[nH]c(-c4ccc(F)cc4)cc3c2)n1] | (R)-N-(2,3-dihydroxypropyl)-6-(2- (4-fluorophenyl)-1H-pyrrolo[2,3- b]pyridin-5-yl)picolinamide | 406.4 |
| 52 | | |
| 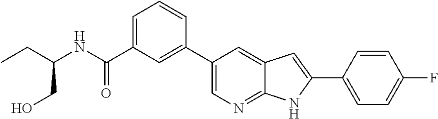 [SMILES: CC[C@H](CO)NC(=O)c1cccc(-c2cnc3[nH]c(-c4ccc(F)cc4)cc3c2)c1] | (R)-3-(2-(4-fluorophenyl)-1H- pyrrolo[2,3-b]pyridin-5-yl)-N-(1- hydroxybutan-2-yl)benzamide | 403.5 |
| 53 | | |
| 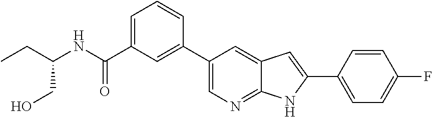 [SMILES: CC[C@@H](CO)NC(=O)c1cccc(-c2cnc3[nH]c(-c4ccc(F)cc4)cc3c2)c1] | (S)-3-(2-(4-fluorophenyl)-1H- pyrrolo[2,3-b]pyridin-5-yl)-N-(1- hydroxybutan-2-yl)benzamide | 403.5 |
| 54 | | |
| 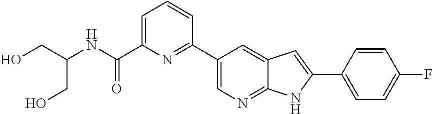 [SMILES: O=C(NC(CO)CO)c1cccc(-c2cnc3[nH]c(-c4ccc(F)cc4)cc3c2)n1] | N-(1,3-dihydroxypropan-2-yl)-6-(2- (4-fluorophenyl)-1H-pyrrolo[2,3- b]pyridin-5-yl)picolinamide | 406.4 |
| 55 | | |
| 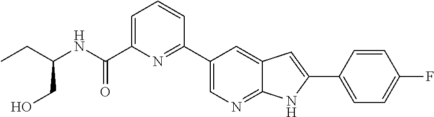 [SMILES: CC[C@H](CO)NC(=O)c1cccc(-c2cnc3[nH]c(-c4ccc(F)cc4)cc3c2)n1] | (R)-6-(2-(4-fluorophenyl)-1H- pyrrolo[2,3-b]pyridin-5-yl)-N-(1- hydroxybutan-2-yl)picolinamide | 404.4 |
| 56 | | |
| 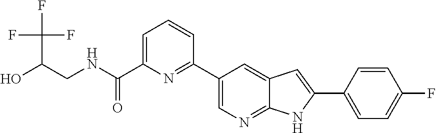 [SMILES: O=C(NCC(O)C(F)(F)F)c1cccc(-c2cnc3[nH]c(-c4ccc(F)cc4)cc3c2)n1] | 6-(2-(4-fluorophenyl)-1H-pyrrolo- [2,3-b]pyridin-5-yl)-N-(3,3,3- trifluoro-2-hydroxypropyl)- picolinamide | 444.4 |
| 57 | | |
| 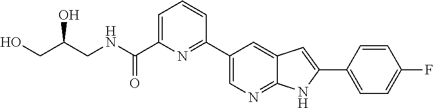 [SMILES: O=C(NC[C@H](O)CO)c1cccc(-c2cnc3[nH]c(-c4ccc(F)cc4)cc3c2)n1] | (S)-N-(2,3-dihydroxypropyl)-6-(2- (4-fluorophenyl)-1H-pyrrolo[2,3- b]pyridin-5-yl)picolinamide | 406.4 |
| 58 | | |
| 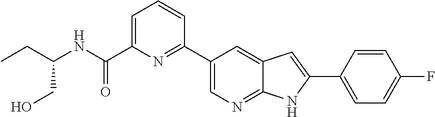 [SMILES: CC[C@@H](CO)NC(=O)c1cccc(-c2cnc3[nH]c(-c4ccc(F)cc4)cc3c2)n1] | (S)-6-(2-(4-fluorophenyl)-1H- pyrrolo[2,3-b]pyridin-5-yl)-N-(1- hydroxybutan-2-yl)picolinamide | 404.4 |
| 59 | | |
| 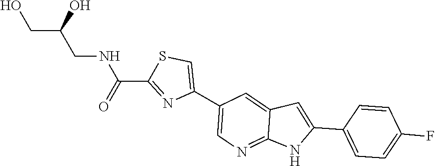 [SMILES: O=C(NC[C@H](O)CO)c1nc(-c2cnc3[nH]c(-c4ccc(F)cc4)cc3c2)cs1] | (S)-N-(2,3-dihydroxypropyl)-4-(2- (4-fluorophenyl)-1H-pyrrolo[2,3- b]pyridin-5-yl)thiazole-2- carboxamide | 412.4 |
| 60 | | |
| 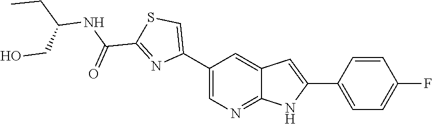 [SMILES: CC[C@@H](CO)NC(=O)c1nc(-c2cnc3[nH]c(-c4ccc(F)cc4)cc3c2)cs1] | (S)-4-(2-(4-fluorophenyl)-1H- pyrrolo[2,3-b]pyridin-5-yl)-N-(1- hydroxybutan-2-yl)thiazole-2- carboxamide | 410.5 |
| 61 | | |
| 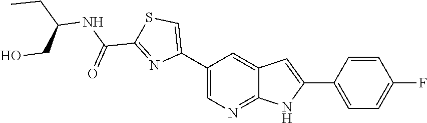 [SMILES: CC[C@H](CO)NC(=O)c1nc(-c2cnc3[nH]c(-c4ccc(F)cc4)cc3c2)cs1] | (R)-4-(2-(4-fluorophenyl)-1H- pyrrolo[2,3-b]pyridin-5-yl)-N-(1- hydroxybutan-2-yl)thiazole-2- carboxamide | 410.5 |
| 62 | | |
| 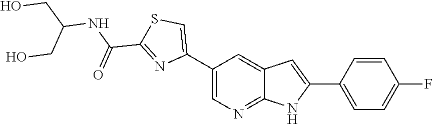 [SMILES: O=C(NC(CO)CO)c1nc(-c2cnc3[nH]c(-c4ccc(F)cc4)cc3c2)cs1] | N-(1,3-dihydroxypropan-2-yl)-4-(2- (4-fluorophenyl)-1H-pyrrolo[2,3- b]pyridin-5-yl)thiazole-2- carboxamide | 412.4 |
| 63 | | |
| 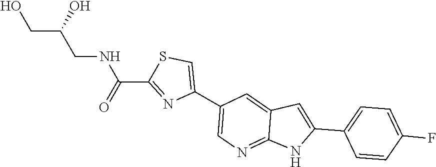 [SMILES: O=C(NC[C@@H](O)CO)c1nc(-c2cnc3[nH]c(-c4ccc(F)cc4)cc3c2)cs1] | (R)-N-(2,3-dihydroxypropyl)-4-(2- (4-fluorophenyl)-1H-pyrrolo[2,3- b]pyridin-5-yl)thiazole-2- carboxamide | 412.4 |
| 64 | | |
| 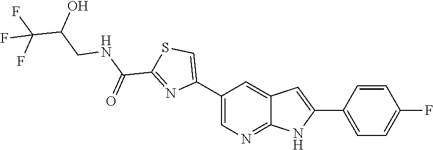 [SMILES: O=C(NCC(O)C(F)(F)F)c1nc(-c2cnc3[nH]c(-c4ccc(F)cc4)cc3c2)cs1] | 4-(2-(4-fluorophenyl)-1H-pyrrolo- [2,3-b]pyridin-5-yl)-N-(3,3,3- trifluoro-2-hydroxypropyl)thiazole- 2-carboxamide | 450.4 |
| 65 | | |
| 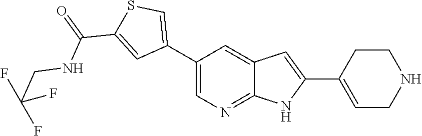 [SMILES: O=C(NCC(F)(F)F)c1cc(-c2cnc3[nH]c(C4=CCNCC4)cc3c2)cs1] | 4-(2-(1,2,3,6-tetrahydropyridin-4- yl)-1H-pyrrolo[2,3-b]pyridin-5-yl)- N-(2,2,2-trifluoroethyl)thiophene-2- carboxamide | 406.4 |
| 66 | | |
| 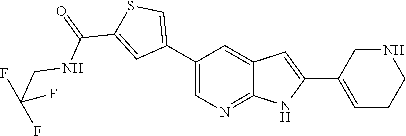 [SMILES: O=C(NCC(F)(F)F)c1cc(-c2cnc3[nH]c(C4=CCCNC4)cc3c2)cs1] | 4-(2-(1,2,5,6-tetrahydropyridin-3- yl)-1H-pyrrolo[2,3-b]pyridin-5-yl)- N-(2,2,2-trifluoroethyl)thiophene-2- carboxamide | 406.4 |
| 67 | | |
| 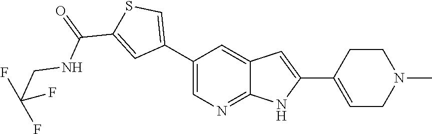 [SMILES: CN1CC=C(c2cc3cc(-c4csc(C(=O)NCC(F)(F)F)c4)cnc3[nH]2)CC1] | 4-(2-(1-methyl-1,2,3,6-tetrahydro- pyridin-4-yl)-1H-pyrrolo[2,3-b]- pyridin-5-yl)-N-(2,2,2-trifluoro- ethyl)thiophene-2-carboxamide | 420.5 |
| 68 | | |
| 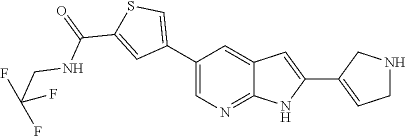 [SMILES: O=C(NCC(F)(F)F)c1cc(-c2cnc3[nH]c(C4=CCNC4)cc3c2)cs1] | 4-(2-(2,5-dihydro-1H-pyrrol-3-yl)- 1H-pyrrolo[2,3-b]pyridin-5-yl)-N- (2,2,2-trifluoroethyl)thiophene-2- carboxamide | 392.4 |
| 69 | | |
| 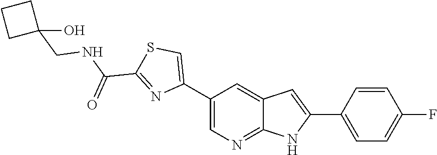 [SMILES: O=C(NCC1(O)CCC1)c1nc(-c2cnc3[nH]c(-c4ccc(F)cc4)cc3c2)cs1] | 4-(2-(4-fluorophenyl)-1H- pyrrolo[2,3-b]pyridin-5-yl)-N-((1- hydroxycyclobutyl)methyl)thiazole- 2-carboxamide | 422.5 |
| 70 | | |
| 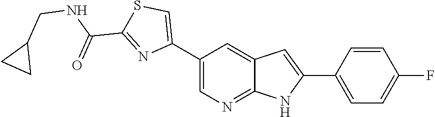 [SMILES: O=C(NCC1CC1)c1nc(-c2cnc3[nH]c(-c4ccc(F)cc4)cc3c2)cs1] | N-(cyclopropylmethyl)-4-(2-(4- fluorophenyl)-1H-pyrrolo[2,3-b]- pyridin-5-yl)thiazole-2- carboxamide | 392.5 |
| 71 | | |
| 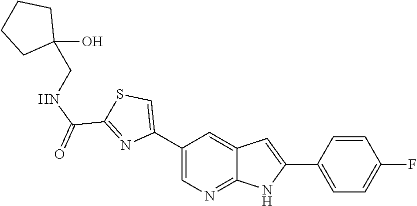 [SMILES: O=C(NCC1(O)CCCC1)c1nc(-c2cnc3[nH]c(-c4ccc(F)cc4)cc3c2)cs1] | 4-(2-(4-fluorophenyl)-1H-pyrrolo- [2,3-b]pyridin-5-yl)-N-((1-hydroxy- cyclopentyl)methyl)thiazole-2- carboxamide | 436.5 |
| 72 | | |
| 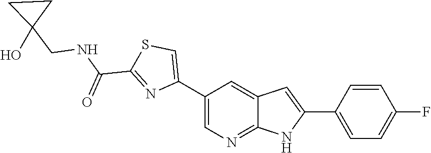 [SMILES: O=C(NCC1(O)CC1)c1nc(-c2cnc3[nH]c(-c4ccc(F)cc4)cc3c2)cs1] | 4-(2-(4-fluorophenyl)-1H-pyrrolo- [2,3-b]pyridin-5-yl)-N-((1-hydroxy- cyclopropyl)methyl)thiazole-2- carboxamide | 408.5 |
| 73 | | |
| 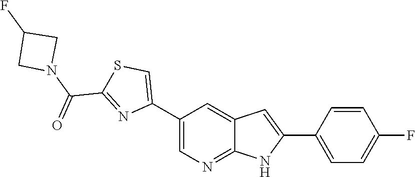 [SMILES: O=C(c1nc(-c2cnc3[nH]c(-c4ccc(F)cc4)cc3c2)cs1)N1CC(F)C1] | (3-fluoroazetidin-1-yl)(4-(2-(4- fluorophenyl)-1H-pyrrolo[2,3-b]- pyridin-5-yl)thiazol-2- yl)methanone | 396.4 |
| 74 | | |
| 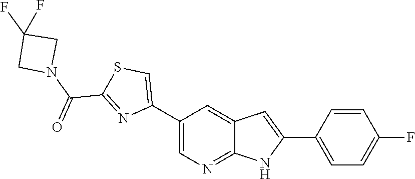 [SMILES: O=C(c1nc(-c2cnc3[nH]c(-c4ccc(F)cc4)cc3c2)cs1)N1CC(F)(F)C1] | (3,3-difluoroazetidin-1-yl)(4-(2-(4- fluorophenyl)-1H-pyrrolo[2,3-b]- pyridin-5-yl)thiazol-2-yl)- methanone | 414.4 |
| 75 | | |
| 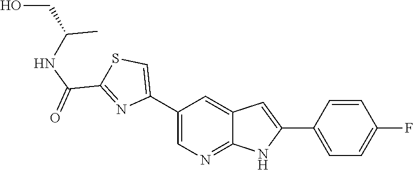 [SMILES: C[C@@H](CO)NC(=O)c1nc(-c2cnc3[nH]c(-c4ccc(F)cc4)cc3c2)cs1] | (S)-4-(2-(4-fluorophenyl)-1H- pyrrolo[2,3-b]pyridin-5-yl)-N-(1- hydroxypropan-2-yl)thiazole-2- carboxamide | 396.4 |
| 76 | | |
| 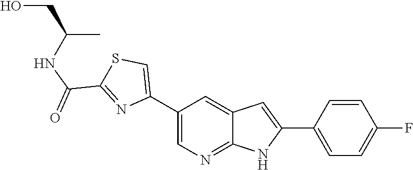 [SMILES: C[C@H](CO)NC(=O)c1nc(-c2cnc3[nH]c(-c4ccc(F)cc4)cc3c2)cs1] | (R)-4-(2-(4-fluorophenyl)-1H- pyrrolo[2,3-b]pyridin-5-yl)-N-(1- hydroxypropan-2-yl)thiazole-2- carboxamide | 396.4 |
| 77 | | |
| 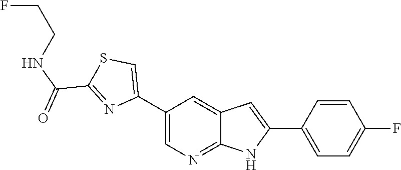 [SMILES: O=C(NCCF)c1nc(-c2cnc3[nH]c(-c4ccc(F)cc4)cc3c2)cs1] | N-(2-fluoroethyl)-4-(2-(4-fluoro- phenyl)-1H-pyrrolo[2,3-b]pyridin- 5-yl)thiazole-2-carboxamide | 384.4 |
| 78 | | |
| 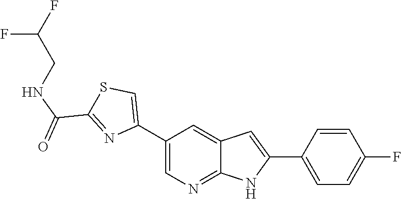 [SMILES: O=C(NCC(F)F)c1nc(-c2cnc3[nH]c(-c4ccc(F)cc4)cc3c2)cs1] | N-(2,2-difluoroethyl)-4-(2-(4- fluorophenyl)-1H-pyrrolo[2,3-b]- pyridin-5-yl)thiazole-2- carboxamide | 402.4 |
| 79 | | |
| 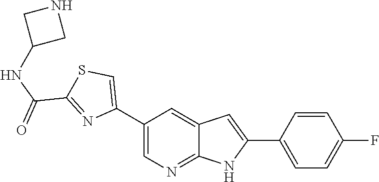 [SMILES: O=C(NC1CNC1)c1nc(-c2cnc3[nH]c(-c4ccc(F)cc4)cc3c2)cs1] | N-(azetidin-3-yl)-4-(2-(4-fluoro- phenyl)-1H-pyrrolo[2,3-b]pyridin- 5-yl)thiazole-2-carboxamide | 393.4 |
| 80 | | |

In another aspect, a compound is provided of Formula II:

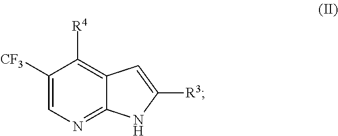

or a pharmaceutically acceptable salt, solvate, prodrug, or derivative thereof;
wherein:
R⁴ is selected from OR⁵ and NR⁶R⁷;
R⁵ is selected from C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
R⁶ is selected from hydrogen, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
R⁷ is selected from C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
or R⁶ and R⁷ are brought together with the nitrogen atom to which they are attached to form a 3- to 8-membered monocyclic or bicyclic heterocycle or a 5- to 10-membered monocyclic or bicyclic heteroaryl, each of which is optionally substituted with one or more groups (for example, 1, 2, 3, or 4 groups) selected from X;
and all other variables are as defined herein.

In some embodiments of Formula II, R⁴ is NR⁶R⁷.

In some embodiments of Formula II, R⁶ is hydrogen.

In some embodiments of Formula II, R⁷ is C₁-C₆ alkyl optionally substituted with 1, 2, 3, or 4 groups selected from X. In some embodiments of Formula II, R⁷ is C₁-C₆ haloalkyl optionally substituted with 1, 2, 3, or 4 groups selected from X. In some embodiments of Formula II, R⁷ is (C₃-C₆ cycloalkyl)(C₀-C₃ alkyl)- optionally substituted with 1, 2, 3, or 4 groups selected from X. In some embodiments of Formula II, R⁷ is (3- to 8-membered monocyclic or bicyclic heterocycle)-(C₀-C₃ alkyl)- optionally substituted with 1, 2, 3, or 4 groups selected from X.

In some embodiments of Formula II, R⁶ and R⁷ are brought together with the nitrogen atom to which they are attached to form a 3- to 8-membered monocyclic or bicyclic heterocycle optionally substituted with 1, 2, 3, or 4 groups selected from X.

In some embodiments of Formula II, R⁴ is selected from:

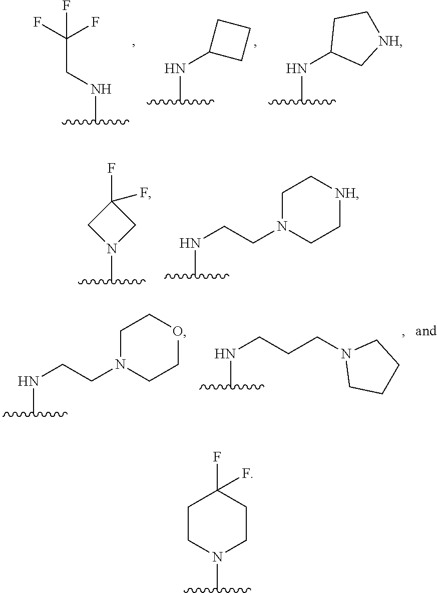

In some embodiments of Formula II, R³ is C₃-C₈ monocyclic or bicyclic cycloalkyl optionally substituted with 1, 2, 3, or 4 groups selected from X. In some embodiments of Formula II, R³ is C₃-C₆ cycloalkenyl optionally substituted with 1, 2, 3, or 4 groups selected from X. In some embodiments of Formula II, R³ is 3- to 8-membered monocyclic or bicyclic heterocycle optionally substituted with 1, 2, 3, or 4 groups selected from X. In some embodiments of Formula II, R³ is 6- to 10-membered monocyclic or bicyclic aryl optionally substituted with 1, 2, 3, or 4 groups selected from X.

In some embodiments of Formula II, R³ is selected from:

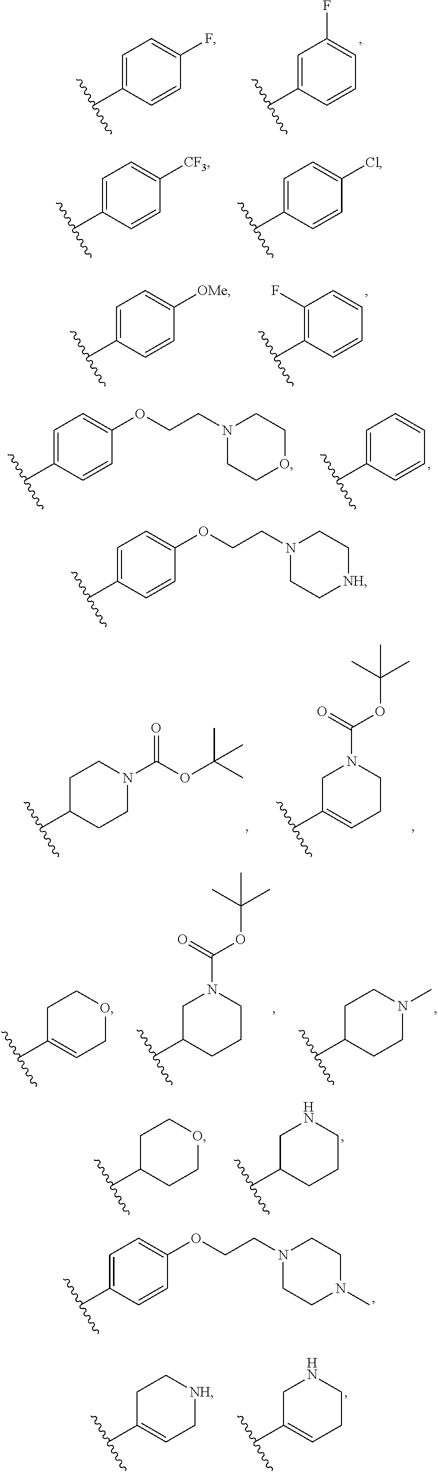

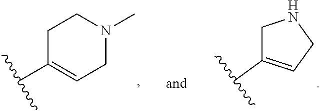

In some embodiments of Formula II, R³ is selected from:

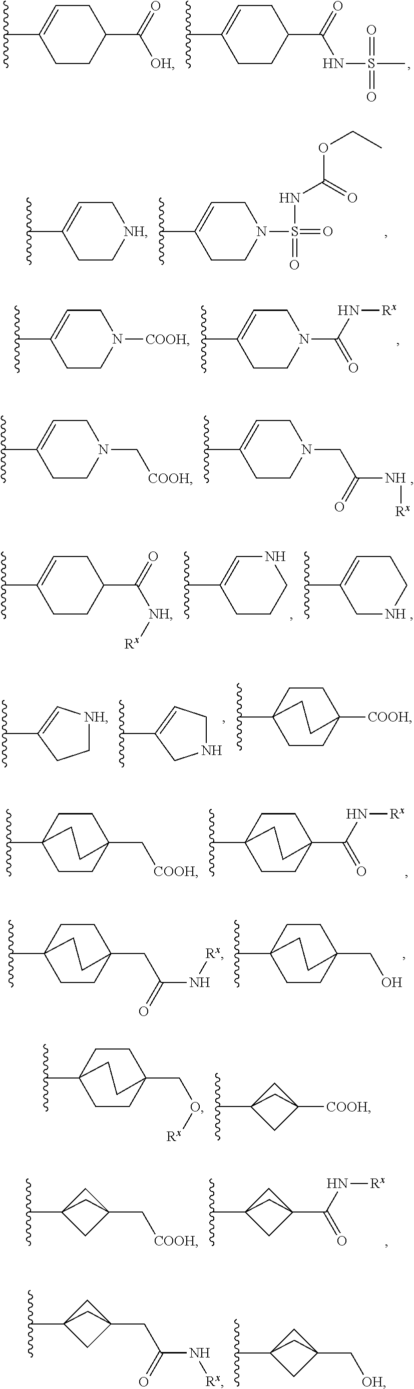

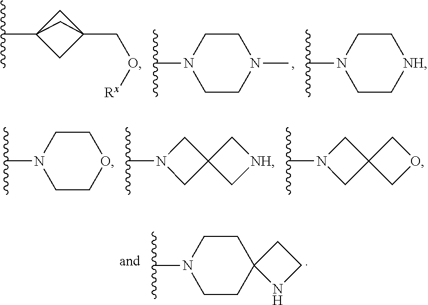

In some embodiments of Formula II, R³ is —B—C.

In some embodiments of Formula II, B is C₃-C₈ monocyclic or bicyclic cycloalkyl optionally substituted with 1, 2, 3, or 4 groups selected from X. In some embodiments of Formula II, B is C₃-C₆ cycloalkenyl optionally substituted with 1, 2, 3, or 4 groups selected from X. In some embodiments of Formula II, B is 3- to 8-membered monocyclic or bicyclic heterocycle optionally substituted with 1, 2, 3, or 4 groups selected from X.

In some embodiments of Formulas II, C is 3- to 8-membered monocyclic or bicyclic heterocycle optionally substituted with 1, 2, 3, or 4 groups selected from X.

In some embodiments of Formula II, R³ is selected from:

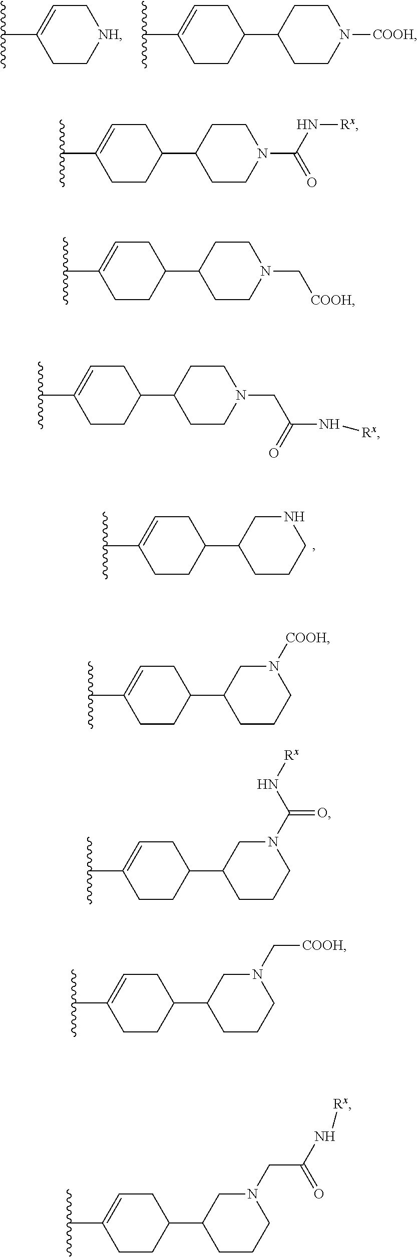

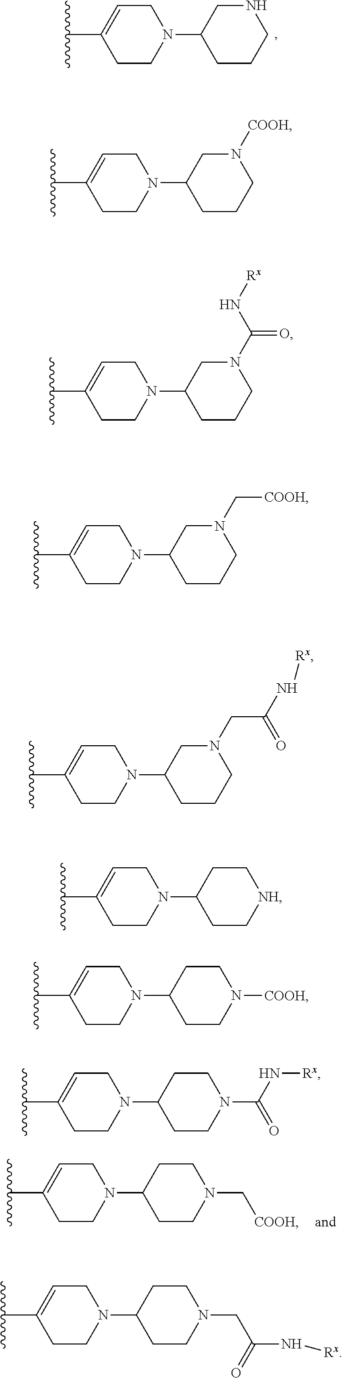

Representative compounds of Formula II include, but are not limited to, the compounds provided in Table 2 below, or pharmaceutically acceptable salts, prodrugs, or derivatives thereof.

TABLE 2
Exemplary Compounds of Formula II

| Structure | | |
|---|---|---|
| Cmpd # | Name | MW |
| 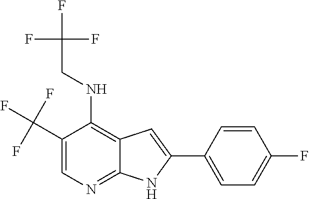 [SMILES: Fc1ccc(-c2cc3c(NCC(F)(F)F)c(C(F)(F)F)cnc3[nH]2)cc1] | 2-(4-fluorophenyl)-N-(2,2,2-trifluoroethyl)- 5-(trifluoromethyl)-1H-pyrrolo[2,3- b]pyridin-4-amine | 377.3 |
| 81 | | |
| 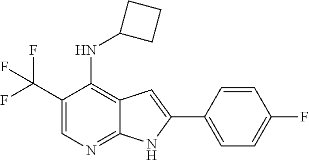 [SMILES: Fc1ccc(-c2cc3c(NC4CCC4)c(C(F)(F)F)cnc3[nH]2)cc1] | N-cyclobutyl-2-(4-fluorophenyl)-5-(trifluoro- methyl)-1H-pyrrolo[2,3-b]pyridin-4-amine | 349.3 |
| 82 | | |
| 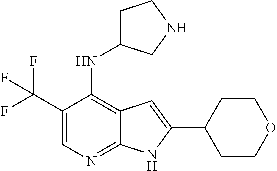 [SMILES: FC(F)(F)c1cnc2[nH]c(C3CCOCC3)cc2c1NC1CCNC1] | N-(pyrrolidin-3-yl)-2-(tetrahydro-2H-pyran- 4-yl)-5-(trifluoromethyl)-1H-pyrrolo[2,3-b]- pyridin-4-amine | 354.4 |
| 83 | | |
| 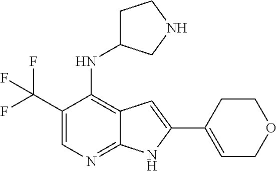 [SMILES: FC(F)(F)c1cnc2[nH]c(C3=CCOCC3)cc2c1NC1CCNC1] | 2-(3,6-dihydro-2H-pyran-4-yl)-N- (pyrrolidin-3-yl)-5-(trifluoromethyl)-1H- pyrrolo[2,3-b]pyridin-4-amine | 352.4 |
| 84 | | |
| 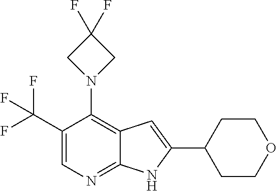 [SMILES: FC1(F)CN(c2c(C(F)(F)F)cnc3[nH]c(C4CCOCC4)cc23)C1] | 4-(3,3-difluoroazetidin-1-yl)-2-(tetrahydro- 2H-pyran-4-yl)-5-(trifluoromethyl)-1H- pyrrolo[2,3-b]pyridine | 361.3 |
| 85 | | |
| 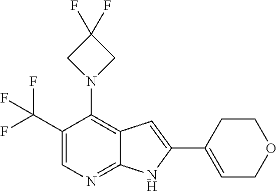 [SMILES: FC1(F)CN(c2c(C(F)(F)F)cnc3[nH]c(C4=CCOCC4)cc23)C1] | 4-(3,3-difluoroazetidin-1-yl)-2-(3,6-dihydro- 2H-pyran-4-yl)-5-(trifluoromethyl)-1H- pyrrolo[2,3-b]pyridine | 359.3 |
| 86 | | |
| 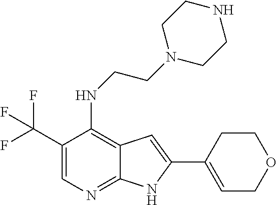 [SMILES: FC(F)(F)c1cnc2[nH]c(C3=CCOCC3)cc2c1NCCN1CCNCC1] | 2-(3,6-dihydro-2H-pyran-4-yl)-N-(2- (piperazin-1-yl)ethyl)-5-(trifluoromethyl)- 1H-pyrrolo[2,3-b]pyridin-4-amine | 395.4 |
| 87 | | |
| 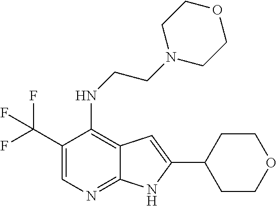 [SMILES: FC(F)(F)c1cnc2[nH]c(C3CCOCC3)cc2c1NCCN1CCOCC1] | N-(2-morpholinoethyl)-2-(tetrahydro-2H- pyran-4-yl)-5-(trifluoromethyl)-1H-pyrrolo- [2,3-b]pyridin-4-amine | 398.4 |
| 88 | | |
| 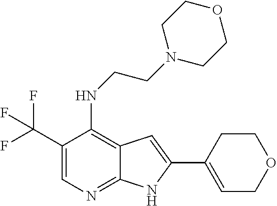 [SMILES: FC(F)(F)c1cnc2[nH]c(C3=CCOCC3)cc2c1NCCN1CCOCC1] | 2-(3,6-dihydro-2H-pyran-4-yl)-N-(2- morpholinoethyl)-5-(trifluoromethyl)-1H- pyrrolo[2,3-b]pyridin-4-amine | 396.4 |
| 89 | | |
| 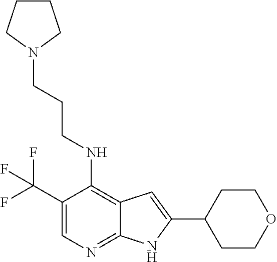 [SMILES: FC(F)(F)c1cnc2[nH]c(C3CCOCC3)cc2c1NCCCN1CCCC1] | N-(3-(pyrrolidin-1-yl)propyl)-2-(tetrahydro- 2H-pyran-4-yl)-5-(trifluoromethyl)-1H- pyrrolo[2,3-b]pyridin-4-amine | 396.5 |
| 90 | | |
| 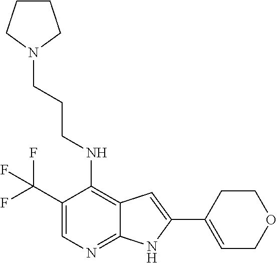 [SMILES: FC(F)(F)c1cnc2[nH]c(C3=CCOCC3)cc2c1NCCCN1CCCC1] | 2-(3,6-dihydro-2H-pyran-4-yl)-N-(3- (pyrrolidin-1-yl)propyl)-5-(trifluoromethyl)- 1H-pyrrolo[2,3-b]pyridin-4-amine | 394.4 |
| 91 | | |
| 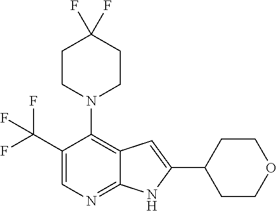 [SMILES: FC1(F)CCN(c2c(C(F)(F)F)cnc3[nH]c(C4CCOCC4)cc23)CC1] | 4-(4,4-difluoropiperidin-1-yl)-2-(tetrahydro- 2H-pyran-4-yl)-5-(trifluoromethyl)-1H- pyrrolo[2,3-b]pyridine | 389.4 |
| 92 | | |
| 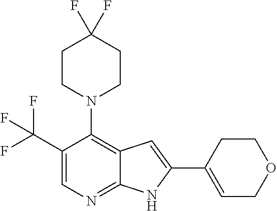 [SMILES: FC1(F)CCN(c2c(C(F)(F)F)cnc3[nH]c(C4=CCOCC4)cc23)CC1] | 4-(4,4-difluoropiperidin-1-yl)-2-(3,6- dihydro-2H-pyran-4-yl)-5-(trifluoromethyl)- 1H-pyrrolo[2,3-b]pyridine | 387.4 |
| 93 | | |
| 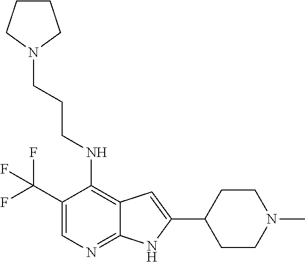 [SMILES: CN1CCC(c2cc3c(NCCCN4CCCC4)c(C(F)(F)F)cnc3[nH]2)CC1] | 2-(1-methylpiperidin-4-yl)-N-(3-(pyrrolidin- 1-yl)propyl)-5-(trifluoromethyl)-1H- pyrrolo[2,3-b]pyridin-4-amine | 409.5 |
| 94 | | |
| 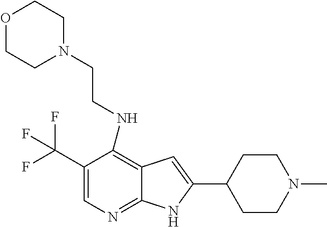 [SMILES: CN1CCC(c2cc3c(NCCN4CCOCC4)c(C(F)(F)F)cnc3[nH]2)CC1] | 2-(1-methylpiperidin-4-yl)-N-(2- morpholinoethyl)-5-(trifluoromethyl)-1H- pyrrolo[2,3-b]pyridin-4-amine | 411.5 |
| 95 | | |
| 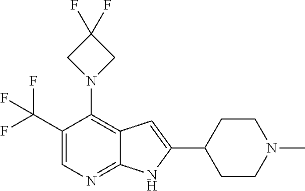 [SMILES: CN1CCC(c2cc3c(N4CC(F)(F)C4)c(C(F)(F)F)cnc3[nH]2)CC1] | 4-(3,3-difluoroazetidin-1-yl)-2-(1- methylpiperidin-4-yl)-5-(trifluoromethyl)- 1H-pyrrolo[2,3-b]pyridine | 374.4 |
| 96 | | |
| 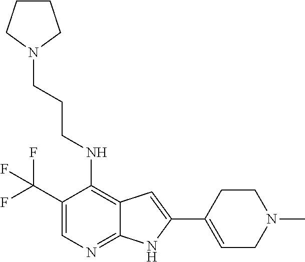 [SMILES: CN1CC=C(c2cc3c(NCCCN4CCCC4)c(C(F)(F)F)cnc3[nH]2)CC1] | 2-(1-methyl-1,2,3,6-tetrahydropyridin-4-yl)- N-(3-(pyrrolidin-1-yl)propyl)-5-(trifluoro- methyl)-1H-pyrrolo[2,3-b]pyridin-4-amine | 407.5 |
| 97 | | |
| 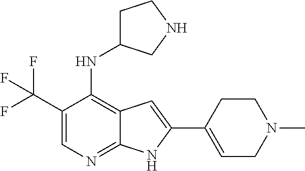 [SMILES: CN1CC=C(c2cc3c(NC4CCNC4)c(C(F)(F)F)cnc3[nH]2)CC1] | 2-(1-methyl-1,2,3,6-tetrahydropyridin-4-yl)- N-(pyrrolidin-3-yl)-5-(trifluoromethyl)-1H- pyrrolo[2,3-b]pyridin-4-amine | 365.4 |
| 98 | | |
| 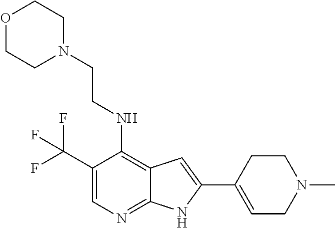 [SMILES: CN1CC=C(c2cc3c(NCCN4CCOCC4)c(C(F)(F)F)cnc3[nH]2)CC1] | 2-(1-methyl-1,2,3,6-tetrahydropyridin-4-yl)- N-(2-morpholinoethyl)-5-(trifluoromethyl)- 1H-pyrrolo[2,3-b]pyridin-4-amine | 409.5 |
| 99 | | |
| 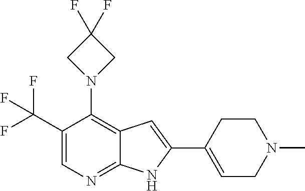 [SMILES: CN1CC=C(c2cc3c(N4CC(F)(F)C4)c(C(F)(F)F)cnc3[nH]2)CC1] | 4-(3,3-difluoroazetidin-1-yl)-2-(1-methyl- 1,2,3,6-tetrahydropyridin-4-yl)-5- (trifluoromethyl)-1H-pyrrolo[2,3-b]pyridine | 372.3 |
| 100 | | |
| 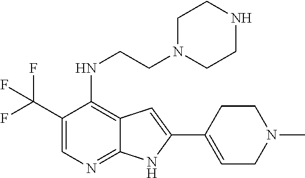 [SMILES: CN1CC=C(c2cc3c(NCCN4CCNCC4)c(C(F)(F)F)cnc3[nH]2)CC1] | 2-(1-methyl-1,2,3,6-tetrahydropyridin-4-yl)- N-(2-(piperazin-1-yl)ethyl)-5- (trifluoromethyl)-1H-pyrrolo[2,3-b]pyridin- 4-amine | 408.5 |
| 101 | | |

Further exemplary compounds of Formula I and Formula II are provided in Table 3 and Table 4 below:

TABLE 3
Further Exemplary Compounds of Formula I

| Structure | Compound # |
|---|---|
| 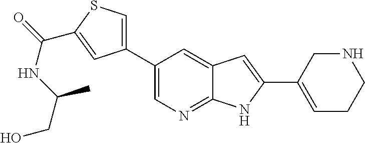 [SMILES: C[C@@H](CO)NC(=O)c1cc(-c2cnc3[nH]c(C4=CCCNC4)cc3c2)cs1] | 102 |
| 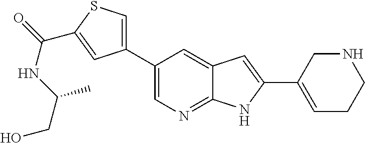 [SMILES: C[C@H](CO)NC(=O)c1cc(-c2cnc3[nH]c(C4=CCCNC4)cc3c2)cs1] | 103 |
| 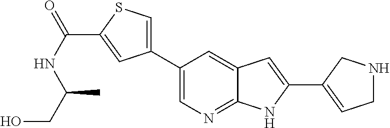 [SMILES: C[C@@H](CO)NC(=O)c1cc(-c2cnc3[nH]c(C4=CCNC4)cc3c2)cs1] | 104 |
| 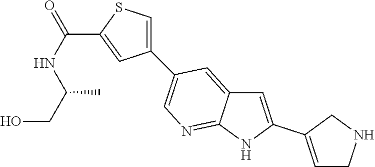 [SMILES: C[C@H](CO)NC(=O)c1cc(-c2cnc3[nH]c(C4=CCNC4)cc3c2)cs1] | 105 |
| 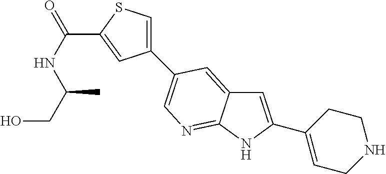 [SMILES: C[C@@H](CO)NC(=O)c1cc(-c2cnc3[nH]c(C4=CCNCC4)cc3c2)cs1] | 106 |
| 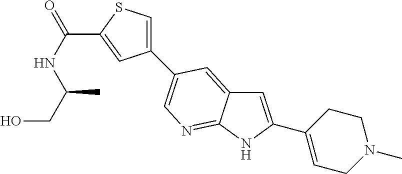 [SMILES: C[C@@H](CO)NC(=O)c1cc(-c2cnc3[nH]c(C4=CCN(C)CC4)cc3c2)cs1] | 107 |
| 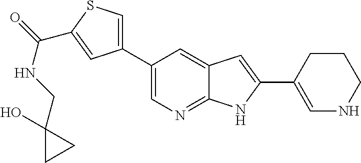 [SMILES: O=C(NCC1(O)CC1)c1cc(-c2cnc3[nH]c(C4=CNCCC4)cc3c2)cs1] | 108 |
| 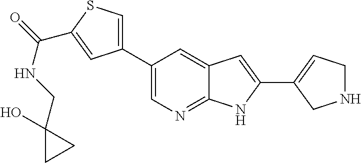 [SMILES: O=C(NCC1(O)CC1)c1cc(-c2cnc3[nH]c(C4=CCNC4)cc3c2)cs1] | 109 |
| 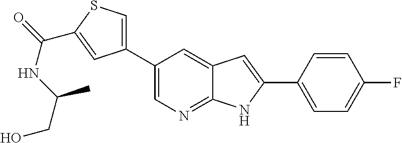 [SMILES: C[C@@H](CO)NC(=O)c1cc(-c2cnc3[nH]c(-c4ccc(F)cc4)cc3c2)cs1] | 110 |
| 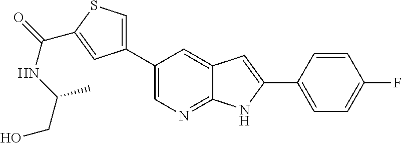 [SMILES: C[C@H](CO)NC(=O)c1cc(-c2cnc3[nH]c(-c4ccc(F)cc4)cc3c2)cs1] | 111 |

TABLE 4
Further Exemplary Compounds of Formula II

| Structure | Compound # |
|---|---|
| 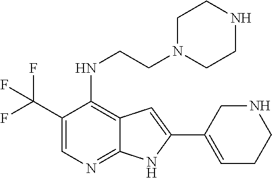 [SMILES: FC(F)(F)c1cnc2[nH]c(C3=CCCNC3)cc2c1NCCN1CCNCC1] | 112 |
| 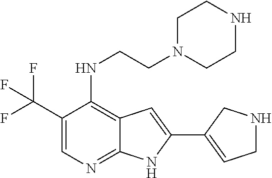 [SMILES: FC(F)(F)c1cnc2[nH]c(C3=CCNC3)cc2c1NCCN1CCNCC1] | 113 |
| 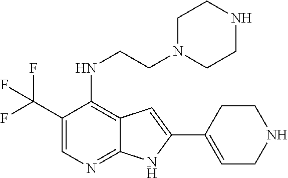 [SMILES: FC(F)(F)c1cnc2[nH]c(C3=CCNCC3)cc2c1NCCN1CCNCC1] | 114 |
| 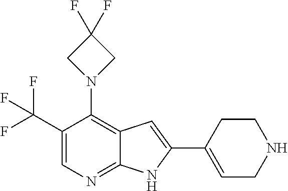 [SMILES: FC1(F)CN(c2c(C(F)(F)F)cnc3[nH]c(C4=CCNCC4)cc23)C1] | 115 |
| 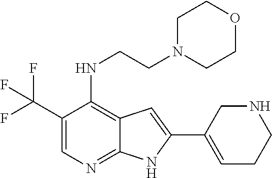 [SMILES: FC(F)(F)c1cnc2[nH]c(C3=CCCNC3)cc2c1NCCN1CCOCC1] | 116 |
| 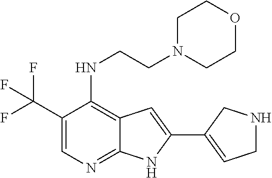 [SMILES: FC(F)(F)c1cnc2[nH]c(C3=CCNC3)cc2c1NCCN1CCOCC1] | 117 |
| 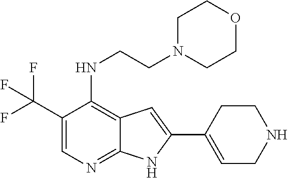 [SMILES: FC(F)(F)c1cnc2[nH]c(C3=CCNCC3)cc2c1NCCN1CCOCC1] | 118 |
| 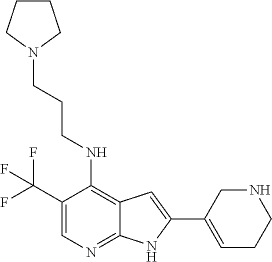 [SMILES: FC(F)(F)c1cnc2[nH]c(C3=CCCNC3)cc2c1NCCCN1CCCC1] | 119 |
| 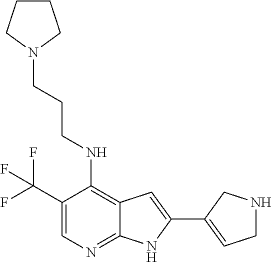 [SMILES: FC(F)(F)c1cnc2[nH]c(C3=CCNC3)cc2c1NCCCN1CCCC1] | 120 |
| 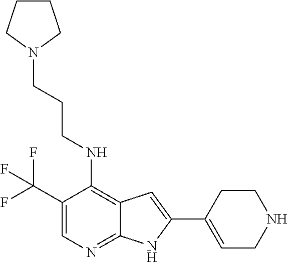 [SMILES: FC(F)(F)c1cnc2[nH]c(C3=CCNCC3)cc2c1NCCCN1CCCC1] | 121 |
| 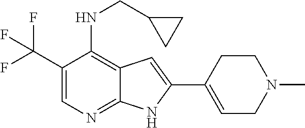 [SMILES: CN1CC=C(c2cc3c(NCC4CC4)c(C(F)(F)F)cnc3[nH]2)CC1] | 122 |
| 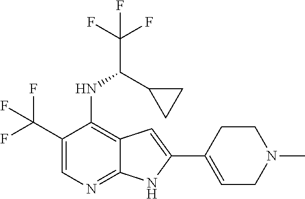 [SMILES: CN1CC=C(c2cc3c(N[C@@H](C4CC4)C(F)(F)F)c(C(F)(F)F)cnc3[nH]2)CC1] | 123 |
| 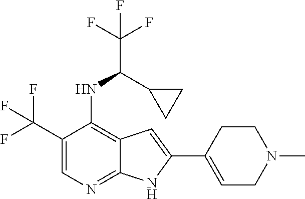 [SMILES: CN1CC=C(c2cc3c(N[C@H](C4CC4)C(F)(F)F)c(C(F)(F)F)cnc3[nH]2)CC1] | 124 |
| 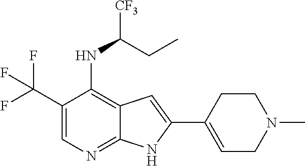 [SMILES: CC[C@@H](Nc1c(C(F)(F)F)cnc2[nH]c(C3=CCN(C)CC3)cc12)C(F)(F)F] | 125 |
| 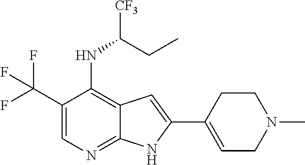 [SMILES: CC[C@H](Nc1c(C(F)(F)F)cnc2[nH]c(C3=CCN(C)CC3)cc12)C(F)(F)F] | 126 |
| 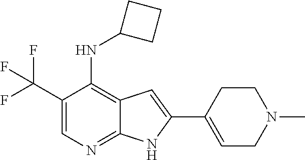 [SMILES: CN1CC=C(c2cc3c(NC4CCC4)c(C(F)(F)F)cnc3[nH]2)CC1] | 127 |
| 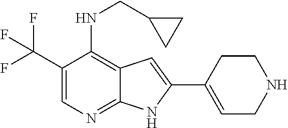 [SMILES: FC(F)(F)c1cnc2[nH]c(C3=CCNCC3)cc2c1NCC1CC1] | 128 |
| 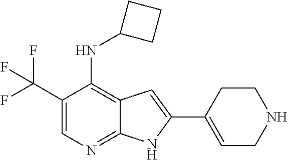 [SMILES: FC(F)(F)c1cnc2[nH]c(C3=CCNCC3)cc2c1NC1CCC1] | 129 |
| 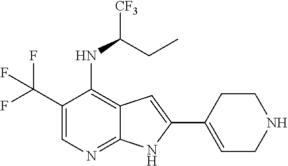 [SMILES: CC[C@@H](Nc1c(C(F)(F)F)cnc2[nH]c(C3=CCNCC3)cc12)C(F)(F)F] | 130 |
| 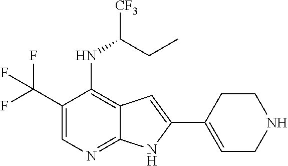 [SMILES: CC[C@H](Nc1c(C(F)(F)F)cnc2[nH]c(C3=CCNCC3)cc12)C(F)(F)F] | 131 |
| 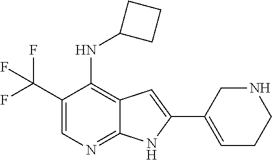 [SMILES: FC(F)(F)c1cnc2[nH]c(C3=CCCNC3)cc2c1NC1CCC1] | 132 |
| 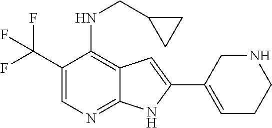 [SMILES: FC(F)(F)c1cnc2[nH]c(C3=CCCNC3)cc2c1NCC1CC1] | 133 |
| 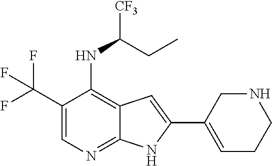 [SMILES: CC[C@@H](Nc1c(C(F)(F)F)cnc2[nH]c(C3=CCCNC3)cc12)C(F)(F)F] | 134 |
| 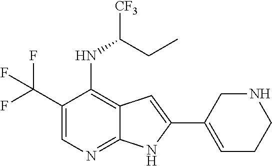 [SMILES: CC[C@H](Nc1c(C(F)(F)F)cnc2[nH]c(C3=CCCNC3)cc12)C(F)(F)F] | 135 |
| 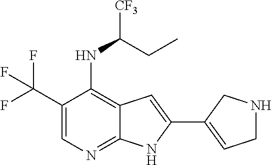 [SMILES: CC[C@@H](Nc1c(C(F)(F)F)cnc2[nH]c(C3=CCNC3)cc12)C(F)(F)F] | 136 |
| 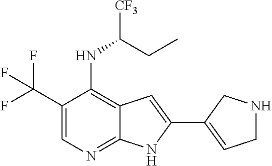 [SMILES: CC[C@H](Nc1c(C(F)(F)F)cnc2[nH]c(C3=CCNC3)cc12)C(F)(F)F] | 137 |
| 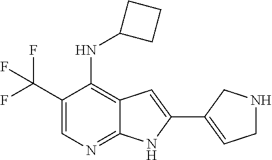 [SMILES: FC(F)(F)c1cnc2[nH]c(C3=CCNC3)cc2c1NC1CCC1] | 138 |
| 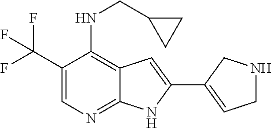 [SMILES: FC(F)(F)c1cnc2[nH]c(C3=CCNC3)cc2c1NCC1CC1] | 139 |
| 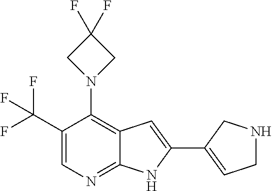 [SMILES: FC1(F)CN(c2c(C(F)(F)F)cnc3[nH]c(C4=CCNC4)cc23)C1] | 140 |
| 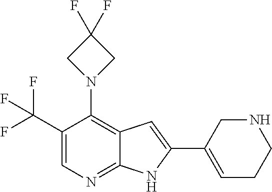 [SMILES: FC1(F)CN(c2c(C(F)(F)F)cnc3[nH]c(C4=CCCNC4)cc23)C1] | 141 |

In an alternative aspect, a compound if provided of Formula Ia or Formula IIa

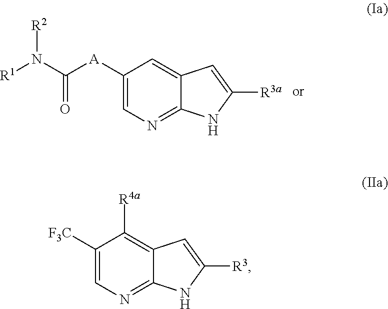

or a pharmaceutically acceptable salt, solvate, prodrug, or derivative thereof,
wherein:
R^{3a} is —B—Ca;
C^{a} is C₃-C₆ cycloalkyl which is optionally substituted with one or more groups (for example, 1, 2, 3, or 4 groups) selected from X;
R^{4a} is C₃-C₆ cycloalkyl, 6- to 10-membered monocyclic or bicyclic aryl, or 5- to 10-membered monocyclic or bicyclic heteroaryl, wherein R^{4a} is optionally substituted with one or more groups selected from X; and
all other variables are as defined herein.

Exemplary compounds of Formula Ia and Formula IIa are provided in Table 5 below:

TABLE 5
Exemplary Compounds of Formula Ia and Formula IIa

| Structure | Compound # |
|---|---|
| 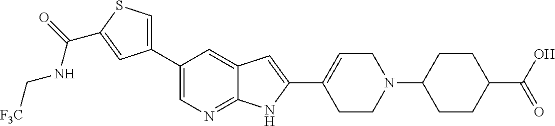 [SMILES: O=C(NCC(F)(F)F)c1cc(-c2cnc3[nH]c(C4=CCN(C5CCC(C(=O)O)CC5)CC4)cc3c2)cs1] | 142 |

The present disclosure also includes compounds of Formula I or Formula II with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P′ ³²P, ³⁵S, ³⁶Cl, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d6-acetone, or d6-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided that has several prodrug moieties in a linear, branched, or cyclic manner.

For example, non-limiting embodiments include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some embodiments, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds as described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art.

They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art.

The dosage can be adjusted by the individual physician in the event of any counterindications. Dosage can vary, and can be administered in one or more dose administrations daily, for one or several days.

### Methods of Use

The present disclosure also provides methods for treating diseases or disorders associated with abnormal cellular proliferation, i.e., proliferative disorders, by administering a compound or composition described herein. The present disclosure also provides methods for treating a disease or disorder mediated by autophagy.

In some embodiments, the proliferative disorder comprises a cancer. Thus, the present disclosure also provides methods for treating or preventing cancer in a subject, comprising administering to the subject a therapeutically effective amount of a compound or composition disclosed herein. The methods can further comprise administering one or more additional therapeutic agents, for example anti-cancer agents or anti-inflammatory agents. Additionally, the method can further comprise administering a therapeutically effective amount of ionizing radiation to the subject.

Methods of killing a cancer or tumor cell are also provided comprising contacting the cancer or tumor cell with an effective amount of a compound or composition as described herein. In some embodiments, the compounds can inhibit ULK1. The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow pig, or horse, or other animals having an oncological disorder. In some aspects, the subject can receive the therapeutic compositions prior to, during, or after surgical intervention to remove part or all of a tumor.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compounds or compositions disclosed herein may comprise carcinomas, sarcomas, lymphomas, leukemias, germ cell tumors, or blastomas.

Carcinomas which may be treated by the compounds or compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compounds or compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compounds or compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenstram macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compounds or compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compounds or compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compounds or compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndromes, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier such as an inert diluent, or an assimilable edible carrier for oral delivery. In addition, the active compound can be incorporated into sustained release preparations and/or devices.

For the treatment of oncological disorder, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor.

For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, imatinid or trastuzumab.

These other substances or radiation treatments can be given at the same time as or at different times from the compounds disclosed herein. Examples of other suitable chemotherapeutic agents include, but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include, but are not limited to, alemtuzumab, cetuximab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein prior to, subsequent to, and/or in combination with administration of a chemotherapeutic agent, an immunotherapeutic agent, a radiotherapeutic agent, or radiotherapy.

In another aspect, the disease or disorder to be treated is a hyperproliferative disorder as further described herein.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, and graft-versus-host rejection.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix.

Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis.

Mesangial disorders are brought about by abnormal proliferation of mesangial cells.

Mesangial hyperproliferative cell disorders include various human renal diseases, such as glomerulonephritis, diabetic nephropathy, malignant nephrosclerosis, thrombotic microangiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other diseases that can include an abnormal cellular proliferative component include Bechet's syndrome, acute respiratory distress syndrome, ischemic heart disease, post-dialysis syndrome, leukemia, acquired immune deficiency syndrome, vasculitis, lipid histiocytosis, septic shock, and inflammation in general.

Additional embodiments of the present disclosure include, but are not limited to: Embodiment 1. A compound of Formula I or Formula II:

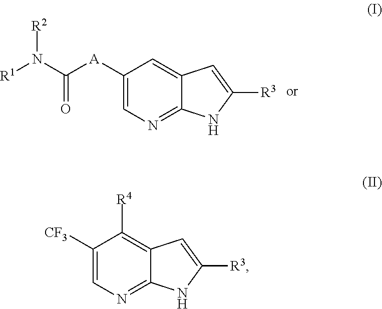

or a pharmaceutically acceptable salt, solvate, prodrug, or derivative thereof, wherein:
A is selected from: 6- to 10-membered monocyclic or bicyclic aryl; and 5- to 10-membered monocyclic or bicyclic heteroaryl; wherein A is optionally substituted with one or more groups (for example, 1, 2, 3, or 4 groups) selected from X; R¹ is selected from C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
R² is selected from hydrogen, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
or R¹ and R² are brought together with the nitrogen atom to which they are attached to form a 3- to 8-membered monocyclic or bicyclic heterocycle or a 5- to 10-membered monocyclic or bicyclic heteroaryl, each of which is optionally substituted with one or more groups (for example, 1, 2, 3, or 4 groups) selected from X;
R³ is selected from: C₃-C₅ monocyclic or bicyclic cycloalkyl; C₃-C₆ cycloalkenyl; 3- to 8-membered monocyclic or bicyclic heterocycle; 6- to 10-membered monocyclic or bicyclic aryl; and 5- to 10-membered monocyclic or bicyclic heteroaryl; wherein R³ is optionally substituted with one or more groups (for example, 1, 2, 3, or 4 groups) selected from X; or
R³ is —B—C;
B is selected from: C₃-C₅ monocyclic or bicyclic cycloalkyl; C₃-C₆ cycloalkenyl; 3- to 8-membered monocyclic or bicyclic heterocycle; wherein B is optionally substituted with one or more groups (for example, 1, 2, 3, or 4 groups) selected from X;
C is 3- to 8-membered monocyclic or bicyclic heterocycle which is optionally substituted with one or more groups (for example, 1, 2, 3, or 4 groups) selected from X;
R⁴ is selected from OR⁵ and NR⁶R⁷;
R⁵ is selected from C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
R⁶ is selected from hydrogen, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
R⁷ is selected from C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
or R⁶ and R⁷ are brought together with the nitrogen atom to which they are attached to form a 3- to 8-membered monocyclic or bicyclic heterocycle or a 5- to 10-membered monocyclic or bicyclic heteroaryl, each of which is optionally substituted with one or more groups (for example, 1, 2, 3, or 4 groups) selected from X;
X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which is optionally substituted with one or more groups (for example, 1, 2, 3, or 4 groups) selected from Y;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{z}, —SR^{z}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

Embodiment 2. A compound of embodiment 1, wherein R³ is C₃-C₈ monocyclic or bicyclic cycloalkyl optionally substituted with 1, 2, 3, or 4 groups selected from X.

Embodiment 3. A compound of embodiment 1, wherein R³ is C₃-C₆ cycloalkenyl optionally substituted with 1, 2, 3, or 4 groups selected from X.

Embodiment 4. A compound of embodiment 1, wherein R³ is 3- to 8-membered monocyclic or bicyclic heterocycle optionally substituted with 1, 2, 3, or 4 groups selected from X.

Embodiment 5. A compound of embodiment 1, wherein R³ is 6- to 10-membered monocyclic or bicyclic aryl optionally substituted with 1, 2, 3, or 4 groups selected from X.

Embodiment 6. A compound of embodiment 1, wherein R³ is selected from:

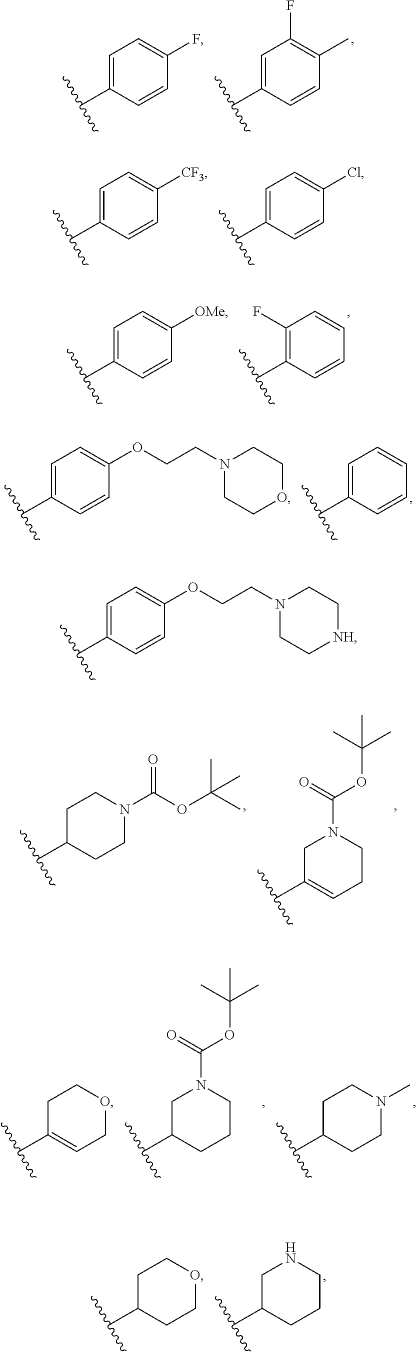

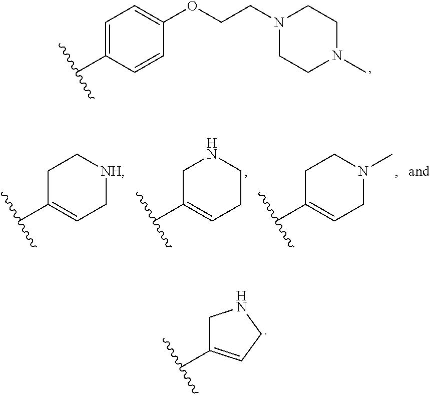

Embodiment 7. A compound of embodiment 1, wherein R³ is selected from:

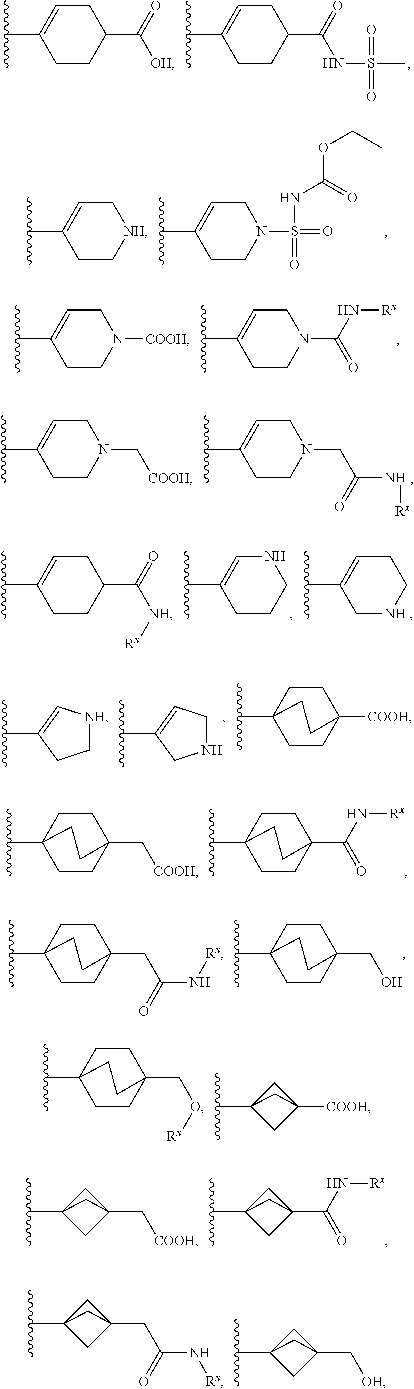

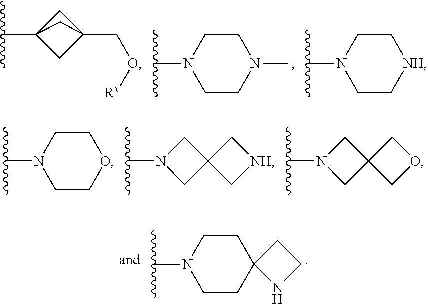

Embodiment 8. A compound of embodiment 1, wherein R³ is —B—C.

Embodiment 9. A compound of embodiment 8, wherein B is C₃-C₈ monocyclic or bicyclic cycloalkyl optionally substituted with 1, 2, 3, or 4 groups selected from X.

Embodiment 10. A compound of embodiment 8, wherein B is C₃-C₆ cycloalkenyl optionally substituted with 1, 2, 3, or 4 groups selected from X.

Embodiment 11. A compound of embodiment 8, wherein B is 3- to 8-membered monocyclic or bicyclic heterocycle optionally substituted with 1, 2, 3, or 4 groups selected from X.

Embodiment 12. A compound of any one of embodiments 8-11, wherein C is 3- to 8-membered monocyclic or bicyclic heterocycle optionally substituted with 1, 2, 3, or 4 groups selected from X.

Embodiment 13 A compound of embodiment 1, wherein R³ is selected from:

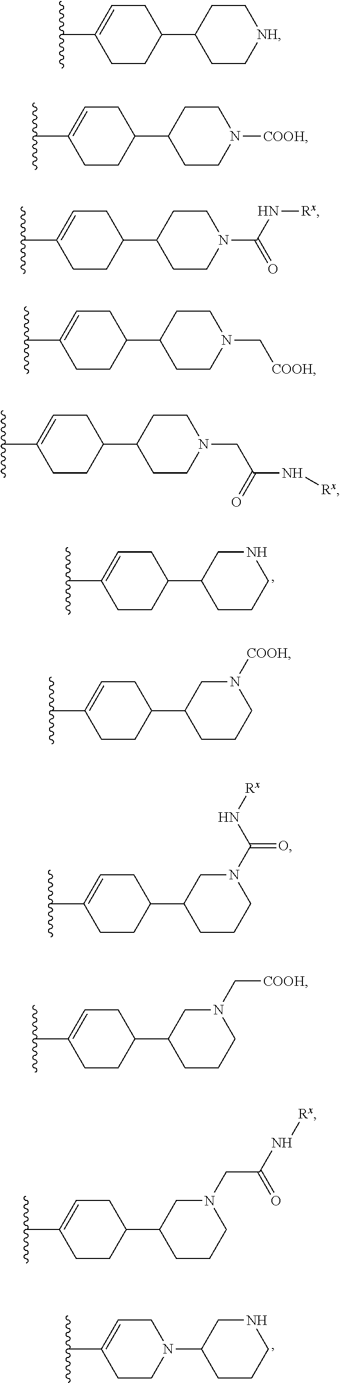

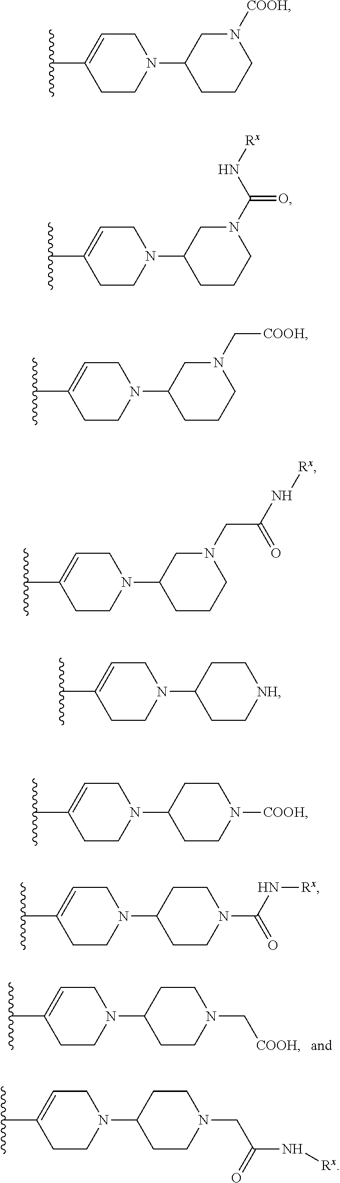

Embodiment 14. A compound of any one of embodiments 1-13, wherein A is 6- to 10-membered monocyclic or bicyclic aryl optionally substituted with 1, 2, 3, or 4 groups selected from X.

Embodiment 15. A compound of any one of embodiments 1-13, wherein A is 5- to 10-membered monocyclic or bicyclic heteroaryl optionally substituted with 1, 2, 3, or 4 groups selected from X.

Embodiment 16. A compound of any one of embodiments 1-13, wherein A is selected from:

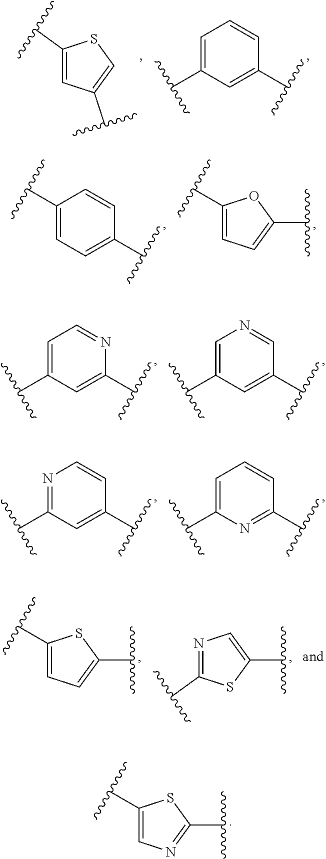

Embodiment 17. A compound of any one of embodiments 1-16, wherein R² is hydrogen.

Embodiment 18. A compound of any one of embodiments 1-17, wherein R¹ is selected from:

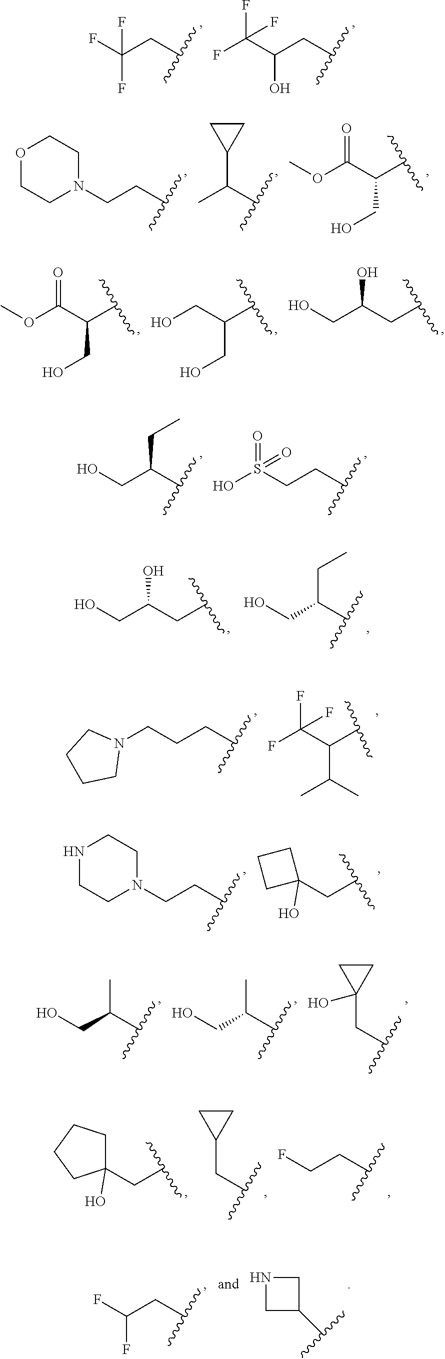

Embodiment 19. A compound of any one of embodiments 1-16, wherein R¹ and R² are brought together with the nitrogen to which they are attached to form a group selected from:

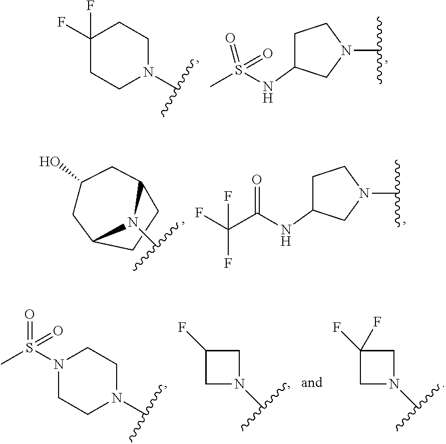

Embodiment 20. A compound of any one of embodiment 1-13, wherein R⁴ is NR⁶R⁷.

Embodiment 21. A compound of embodiment 20, wherein R⁶ is hydrogen.

Embodiment 22. A compound of embodiment 20 or embodiment 21, wherein R⁷ is C₁-C₆ alkyl optionally substituted with 1, 2, 3, or 4 groups selected from X.

Embodiment 23. A compound of embodiment 20 or embodiment 21, wherein R⁷ is C₁-C₆ haloalkyl optionally substituted with 1, 2, 3, or 4 groups selected from X.

Embodiment 24. A compound of embodiment 20 or embodiment 21, wherein R⁷ is (C₃-C₆ cycloalkyl)(C₀-C₃ alkyl)- optionally substituted with 1, 2, 3, or 4 groups selected from X.

Embodiment 25. A compound of embodiment 20 or embodiment 21, wherein R⁷ is (3- to 8-membered monocyclic or bicyclic heterocycle)-(C₀-C₃ alkyl)- optionally substituted with 1, 2, 3, or 4 groups selected from X.

Embodiment 26. A compound of embodiment 20, wherein R⁶ and R⁷ are brought together with the nitrogen atom to which they are attached to form a 3- to 8-membered monocyclic or bicyclic heterocycle optionally substituted with 1, 2, 3, or 4 groups selected from X.

Embodiment 27. A compound of any one of embodiments 1-13, wherein R⁴ is selected from:

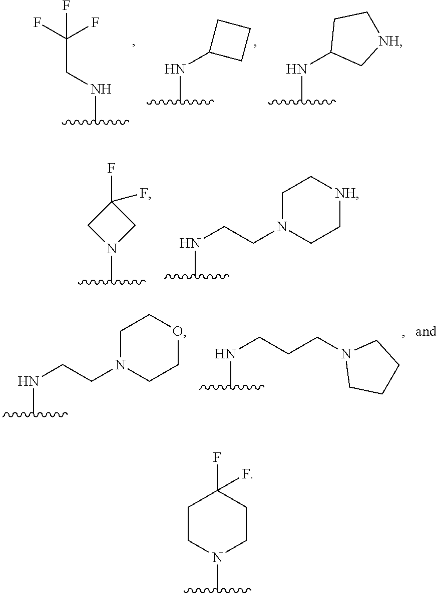

Embodiment 28. A compound of Formula Ia or Formula IIa

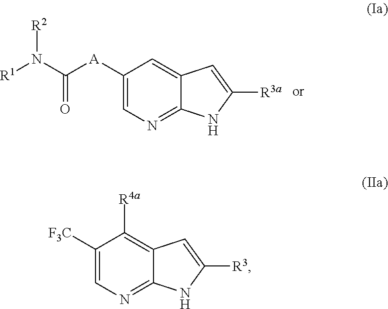

or a pharmaceutically acceptable salt, solvate, prodrug, or derivative thereof,
wherein:
A is selected from: 6- to 10-membered monocyclic or bicyclic aryl; and 5- to 10-membered monocyclic or bicyclic heteroaryl; wherein A is optionally substituted with one or more groups (for example, 1, 2, 3, or 4 groups) selected from X;
R¹ is selected from C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
R² is selected from hydrogen, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups (for example, 1, 2, 3, or 4 groups) selected from X;
or R¹ and R² are brought together with the nitrogen atom to which they are attached to form a 3- to 8-membered monocyclic or bicyclic heterocycle or a 5- to 10-membered monocyclic or bicyclic heteroaryl, each of which is optionally substituted with one or more groups (for example, 1, 2, 3, or 4 groups) selected from X;
R³ is selected from: C₃-C₈ monocyclic or bicyclic cycloalkyl; C₃-C₆ cycloalkenyl; 3- to 8-membered monocyclic or bicyclic heterocycle; 6- to 10-membered monocyclic or bicyclic aryl; and 5- to 10-membered monocyclic or bicyclic heteroaryl; wherein R³ is optionally substituted with one or more groups (for example, 1, 2, 3, or 4 groups) selected from X; or
R³ is —B—C;
B is selected from: C₃-C₈ monocyclic or bicyclic cycloalkyl; C₃-C₆ cycloalkenyl; 3- to 8-membered monocyclic or bicyclic heterocycle; wherein B is optionally substituted with one or more groups (for example, 1, 2, 3, or 4 groups) selected from X;
C is 3- to 8-membered monocyclic or bicyclic heterocycle which is optionally substituted with one or more groups (for example, 1, 2, 3, or 4 groups) selected from X;
R^{3a} is —B—Ca;
C^{a} is C₃-C₆ cycloalkyl which is optionally substituted with one or more groups (for example, 1, 2, 3, or 4 groups) selected from X;
R^{4a} is C₃-C₆ cycloalkyl, 6- to 10-membered monocyclic or bicyclic aryl, or 5- to 10-membered monocyclic or bicyclic heteroaryl, wherein R^{4a} is optionally substituted with one or more groups selected from X;
X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which is optionally substituted with one or more groups (for example, 1, 2, 3, or 4 groups) selected from Y;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

Embodiment 29. A compound selected from a compound listed in Table 1, Table 2, Table 3, Table 4, or Table 5, or a pharmaceutically acceptable salt, solvate, prodrug, or derivative thereof.

Embodiment 30. A pharmaceutical composition comprising a compound of any one of embodiments 1-29, or a pharmaceutically acceptable salt, solvate, prodrug, or derivative thereof, and a pharmaceutically acceptable carrier or excipient.

Embodiment 31. A method of treating a disorder associated with abnormal cellular proliferation in a subject in need thereof comprising administering to the subject a therapeutically effective amount of a compound of any one of embodiments 1-29, or a pharmaceutically acceptable salt, solvate, prodrug, or derivative thereof, or a pharmaceutical composition of embodiment 30.

Embodiment 32. The method of embodiment 31, wherein the disorder comprises a cancer.

Embodiment 33. The method of embodiment 32, wherein the cancer is selected from lung cancer, pancreatic cancer, colorectal cancer, leukemias, lymphoma, melanoma, multiple myeloma, Ewing's sarcoma, osteosarcoma, gastric adenocarcinoma, cervical adenocarcinoma, and head and neck squamous cell carcinoma.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Synthesis of ULK1 Inhibitors

General. All reagents were purchased from commercial suppliers and were used without further purification. Dichloromethane, diethyl ether, N,N-dimethylformamide and tetrahydrofuran were dried by being passed through a column of desiccant (activated A-1 alumina). Triethylamine and diisopropyl amine were purified by distillation from calcium hydride. Reactions were either monitored by thin layer chromatography or analytical LC-MS. Thin layer chromatography was performed on Kieselgel 60 F254 glass plates pre-coated with a 0.25 mm thickness of silica gel. TLC plates were visualized with UV light and/or by staining with ninhydrin solution. Normal phase column chromatography was performed on a Biotage Selekt automated flash system. Compounds were loaded onto pre-filled cartridges filled with KP-Sil 50 μm irregular silica. For microwave reactions, a Biotage Initiator Microwave system was used. Some of the final products were isolated by reverse-phase HPLC using Waters HPLC system with UV detector, with Atlantis T3 OBD Prep Column, 100 Å, 5 μm, 19 mm×150 mm. Compounds were eluted using a gradient elution of 90/10 to 0/100 A/B over 20 min at a flow rate of 20.0 m/min, where solvent A was water (+0.1% ammonium acetate) and solvent B was acetonitrile.

The structures of all compounds were verified via ¹H NMR, ¹³C NMR, ¹⁹F NMR and LCMS. The purity of isolated products was determined using an LC-MS instrument (Agilent 1290 Infinity series LC with single quadrupole MSD system, AP-ESI Ion Source) equipped with Kinetex® 1.7 μm C18 100 Å, LC Column 50×2.1 mm, Ea (Phenomenex) column. Elution was performed using the following conditions: 2% (v/v) acetonitrile (+0.1% FA) in 98% (v/v) H₂O (+0.1% FA), ramped to 98% acetonitrile over 4.0 min, and holding at 98% acetonitrile for 0.5 min with a flow rate of 0.6 mL/min; UV absorption was detected from 200 to 950 nm using a diode array detector. The purity of each compound was >95% based on this analysis.

NMR spectra were recorded at ambient temperature on a 500 MHz Bruker NMR spectrometer in DMSO-d6. All ¹H NMR data are reported in parts per million (ppm) downfield of TMS and were measured relative to the signals for dimethyl sulfoxide (2.50 ppm). All ¹³C NMR spectra are reported in ppm relative to the signals for dimethyl sulfoxide (39.5 ppm) with ¹H decoupled observation. ¹⁹F NMR experiments were performed with ¹H decoupling. Data for ¹H NMR are reported as follows: chemical shift (6, ppm), multiplicity (s=singlet, d=doublet, t=triplet, q=quartet, m=multiplet), integration, and coupling constant (Hz), whereas ¹³C NMR analyses were obtained at 125 Mhz and reported in terms of chemical shift. NMR data was analyzed and processed by using MestReNova software.

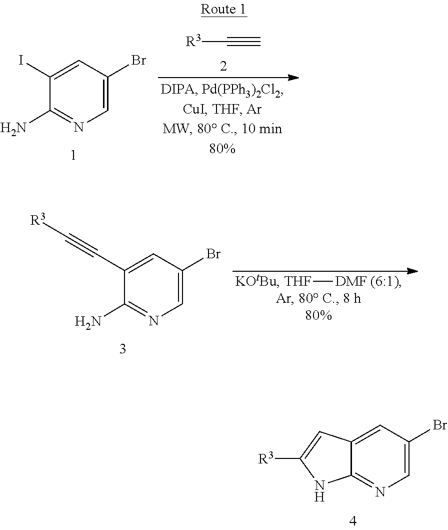

### 5-Bromo-3-((4-fluorophenyl) ethynyl)pyridin-2-amine (3a)

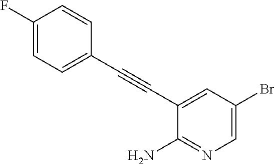 [SMILES: Nc1ncc(Br)cc1C#Cc1ccc(F)cc1]

To a flame-dry microwave vial, 15 ml of dry THF was added. The solvent was degassed via ultrasonication under Ar for 5 minutes. 5-Bromo-3-iodopyridin-2-amine (1) (1 eqv., 3.35 mmol, 1 g), 1-ethynyl-4-fluorobenzene (1.3 eqv., 4.35 mmol, 545 mg, d=1.06 g/ml, 493 mml), copper(I) iodide (0.04 eqv., 0.13 mmol, 25 mg) and DIPA (2.4 eqv., 8.03 mmol, d=0.772 g/ml, 1.13 ml) were added and the mixture was degassed via ultrasonication a second time (5 min). Bis(triphenylphospine) palladium(II) dichloride (0.04 eqv., 0.13 mmol, 94 mg) was then added, and the vial was sealed, degassed via ultrasonication for another 5 minutes and heated at 80° C. for 10 minutes under microwave irradiation. The formation of the product was confirmed by TLC (Hexane 70%, Ethyl acetate 30%) rf: 0.46. The reaction mixture was concentrated under reduced pressure, put on Isolute HM-N celite, and purified via flash chromatography (silica, hexane-ethyl acetate gradient, 5% EtOAc 1 CV, 10 CV gradient from 5% EtOAc to 60% EtOAc).

Yield 80% (775 mg). ¹H NMR (500 MHz, CDCl₃) δ 8.09 (s, 1H), 7.70 (d, J=2.3 Hz, 1H), 7.55-7.45 (m, 2H), 7.27 (s, 2H), 7.14-7.03 (m, 2H), 5.11 (s, 2H). LCMS (m/z): 291 (M+1), Rₜ: 3.305 min.

### 5-Bromo-2-(4-fluorophenyl)-1H-pyrrolo[2,3-b]pyridine (4a)

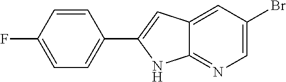 [SMILES: Fc1ccc(-c2cc3cc(Br)cnc3[nH]2)cc1]

To a Schlenk tube, a solution of 5-bromo-3-((4-fluorophenyl)ethynyl)pyridin-2-amine (3a) (1 eqv., 1.03 mmol, 300 mg) in THF (3 ml), potassium tert-butoxide (2 eqv., 2.06 mmol, 231 mg) and DMF (0.5 ml) was added under Ar. The reaction mixture was heated at 80° C. for 8 hours. After the reaction was finished, the solvent was removed under reduced pressure and the reaction mixture put on Isolute HM-N celite, and purified via flash chromatography (silica, hexane-ethyl acetate gradient, 12% EtOAc 1 CV, 10 CV gradient from 12% EtOAc to 100% EtOAc).

Yield 80% (239 mg). ¹H NMR (500 MHz, DMSO-d₆) δ 12.38 (s, 1H), 8.26 (d, J=2.2 Hz, 1H), 8.17 (d, J=2.2 Hz, 1H), 8.07-7.91 (m, 2H), 7.42-7.22 (m, 2H), 6.90 (d, J=2.2 Hz, 1H). LCMS (m/z): 291 (M+1), Rₜ: 3.624 min.

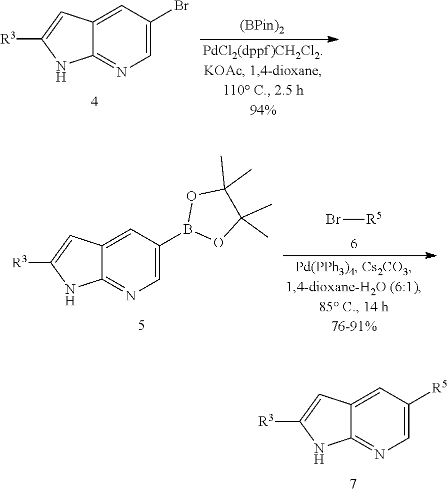

### 2-(4-Fluorophenyl)-5-(4,4,5,5-tetramethyl-1,3,2-dioxaborolan-2-yl)-1H-pyrrolo[2,3-b]-pyridine (5a)

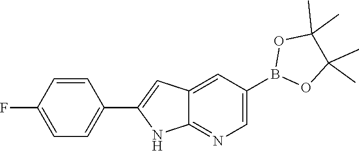 [SMILES: CC1(C)OB(c2cnc3[nH]c(-c4ccc(F)cc4)cc3c2)OC1(C)C]

To a flame dried Schlenk, tube 5-bromo-2-(4-fluorophenyl)-1H-pyrrolo[2,3-b]pyridine (4a) (1 eqv., 5.84 mmol, 1.70 g), 4,4,4′,4′,5,5,5′,5′-octamethyl-2,2′-bi(1,3,2-dioxaborolane) (1.13 eqv., 6.60 mmol, 1.68 g, potassium acetate (3.9 eqv., 22.77 mmol, 2.24 g), 1,1′-bis-(diphenylphosphino)-ferrocene]-dichloropalladium(II) (0.1 eqv., 584 umol, 477 mg) were added. After the addition of the 1,4-dioxane (60 ml) the flask was filled with Ar, closed and heated for 110° C. for 3 h. After the reaction was finished, the solvent was removed under reduced pressure, reaction mixture put on Isolute HM-N celite, and purified via flash chromatography (silica, hexane-ethyl acetate gradient, 12% EtOAc 1CV, 10 CV gradient from 12% EtOAc to 100% EtOAc).

Yield 879 mg (45%). ¹H{¹⁹F} NMR (500 MHz, DMSO-d₆) δ 12.29 (s, 1H), 8.45 (d, J=1.5 Hz, 1H), 8.19 (d, J=1.6 Hz, 1H), 8.01-7.94 (m, 2H), 7.35-7.30 (m, 2H), 6.94 (d, J=2.0 Hz, 1H), 1.32 (s, 12H). ¹⁹F{¹H} NMR (471 MHz, DMSO-d₆) δ−113.51 (s, 1F). LCMS (m/z): 339 (M+1), Rₜ: 3.384 min.

### Methyl 4-(2-(4-fluorophenyl)-1H-pyrrolo[2,3-b]pyridin-5-yl)thiophene-2-carboxylate (7a)

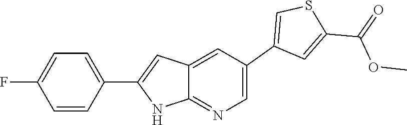 [SMILES: COC(=O)c1cc(-c2cnc3[nH]c(-c4ccc(F)cc4)cc3c2)cs1]

To a Schlenk tube, 2-(4-fluorophenyl)-5-(4,4,5,5-tetramethyl-1,3,2-dioxaborolan-2-yl)-1H-pyrrolo[2,3-b]pyridine (5a) (1 eqv., 2.96 mmol. 1.00 g), methyl 4-bromothiophene-2-carboxylate (6a) (1 eqv., 2.96 mmol, 654 mg), tetrakis(triphenylphosphine)palladium(0) (0.05 eqv., 148 umol, 171 mg), cesium carbonate (1.6 eqv., 4.73 mmol, 1.54 g) were added. The flask was backfilled with Ar and 1,4-dioxane (48 ml) and water (2.4 ml) were added. The flask was closed and heated at 85° C. for 14 hours. After the reaction was finished, the solvent was removed under reduced pressure, 2 ml of water was added. The mixture was ultrasonicated for 2 minutes to dissolve cesium carbonate and solid was filtered off, washed with water, and dried on air. The intermediate was used in the next step without further purification.

Yield 81% (849 mg). LCMS (m/z): 353 (M+1), Rₜ: 3.304 min.

### Methyl 6-(2-(4-fluorophenyl)-1H-pyrrolo[2,3-b]pyridin-5-yl)picolinate (7b)

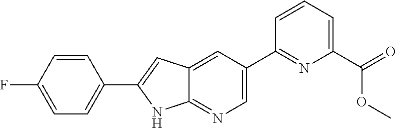 [SMILES: COC(=O)c1cccc(-c2cnc3[nH]c(-c4ccc(F)cc4)cc3c2)n1]

Synthesized accordingly procedure described for 7a.

Yield 76% (389 mg). LCMS (m/z): 348 (M+1), Rₜ: 2.932 min.

### Ethyl 4-(2-(4-fluorophenyl)-1H-pyrrolo[2,3-b]pyridin-5-yl)thiazole-2-carboxylate (7c)

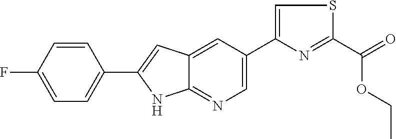 [SMILES: CCOC(=O)c1nc(-c2cnc3[nH]c(-c4ccc(F)cc4)cc3c2)cs1]

Synthesized accordingly procedure, described for 7a.

Yield 91% (492 mg). LCMS (m/z): 368 (M+1), Rₜ: 3.146 min.

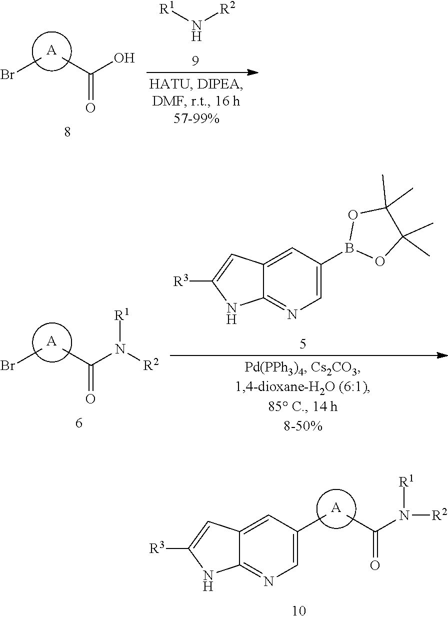

### 5-Bromo-N-(2,2,2-trifluoroethyl)thiophene-2-carboxamide (6b)

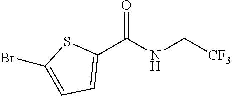 [SMILES: O=C(NCC(F)(F)F)c1ccc(Br)s1]

The 5-bromothiophene-2-carboxylic acid (8a) (1 eqv., 1.21 mmol, 250 mg), 2,2,2-trifluoroethan-1-amine (9a) (1.2 eqv., 1.45 mmol, 144 mg), HATU (2 eqv., 2.41 mmol, 918 mg), anhydrous DIPEA (3 eqv., 3.62 mmol, d=0.742 g/ml, 0.631 ml), DMF (5 ml) were put in a flame dried flask. The flask was filled with Ar, sealed, and left for 14 h. After the reaction was finished, the solvent was removed under reduced pressure, reaction mixture put on Isolute HM-N celite, and purified via flash chromatography (silica, hexane-ethyl acetate gradient, 12% EtOAc 1 CV, 10 CV gradient from 12% EtOAc to 100% EtOAc). Yield 57% (200 mg). LCMS (m/z): 287 (M+1), Rₜ: 2.716 min.

### 5-Bromo-N-(2,2,2-trifluoroethyl)furan-2-carboxamide (6c)

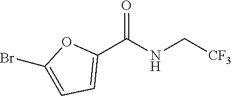 [SMILES: O=C(NCC(F)(F)F)c1ccc(Br)o1]

Synthesized accordingly procedure, described for 6b.

Yield 90% (296 mg). LCMS (m/z): 272 (M+1), Rₜ: 2.403 min.

### 5-Bromo-N-(2,2,2-trifluoroethyl)thiazole-2-carboxamide (6d)

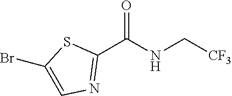 [SMILES: O=C(NCC(F)(F)F)c1ncc(Br)s1]

Synthesized accordingly procedure, described for 6b.

Yield 82% (286 mg). LCMS (m/z): 289 (M+1), Rₜ: 2.602 min.

### 2-Bromo-N-(2,2,2-trifluoroethyl)thiazole-5-carboxamide (6e)

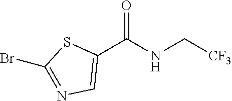 [SMILES: O=C(NCC(F)(F)F)c1cnc(Br)s1]

Synthesized accordingly procedure, described for 6b.

Yield 90% (296 mg). LCMS (m/z): 289 (M+1), Rₜ: 2.382 min.

### 2-Bromo-N-(2,2,2-trifluoroethyl)isonicotinamide (6f)

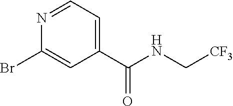 [SMILES: O=C(NCC(F)(F)F)c1ccnc(Br)c1]

Synthesized accordingly procedure, described for 6b.

Yield 99% (340 mg). LCMS (m/z): 283 (M+1), Rₜ: 2.295 min.

### 5-bromo-N-(2,2,2-trifluoroethyl)nicotinamide (6g)

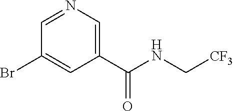 [SMILES: O=C(NCC(F)(F)F)c1cncc(Br)c1]

Synthesized accordingly procedure, described for 6b.

Yield 76% (260 mg). LCMS (m/z): 283 (M+1), Rₜ: 2.278 min.

### 4-bromo-N-(2,2,2-trifluoroethyl)picolinamide (6h)

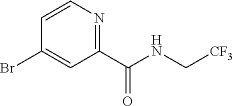 [SMILES: O=C(NCC(F)(F)F)c1cc(Br)ccn1]

Synthesized accordingly procedure, described for 6b.

Yield 91% (310 mg). LCMS (m/z): 283 (M+1), Rₜ: 2.665 min.

### 6-bromo-N-(2,2,2-trifluoroethyl)picolinamide (6i)

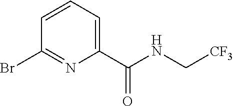 [SMILES: O=C(NCC(F)(F)F)c1cccc(Br)n1]

Synthesized accordingly procedure, described for 6b.

Yield 93% (317 mg). LCMS (m/z): 283 (M+1), Rₜ: 2.631 min.

### 4-Bromo-N-(2,2,2-trifluoroethyl)thiophene-2-carboxamide (6k)

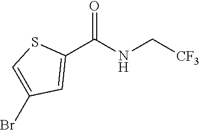 [SMILES: O=C(NCC(F)(F)F)c1cc(Br)cs1]

Synthesized accordingly procedure, described for 6b.

Yield 90% (2.50 g). LCMS (m/z): 288 (M+1), Rₜ: 2.672 min.

### 5-(2-(4-Fluorophenyl)-1H-pyrrolo[2,3-b]pyridin-5-yl)-N-(2,2,2-trifluoroethyl)-thiophene-2-carboxamide (10a) (Compound 20)

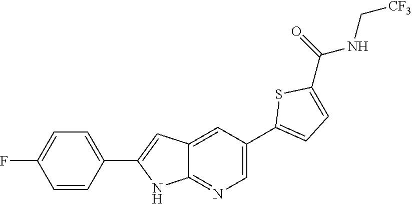 [SMILES: O=C(NCC(F)(F)F)c1ccc(-c2cnc3[nH]c(-c4ccc(F)cc4)cc3c2)s1]

Synthesized accordingly procedure, described for 7a.

Yield 8% (5 mg). ¹H NMR (500 MHz, DMSO-d₆) δ 12.40 (s, 1H), 9.18 (t, J=6.2 Hz, 1H), 8.61 (d, J=2.2 Hz, 1H), 8.26 (d, J=2.2 Hz, 1H), 8.05-7.98 (m, 2H), 7.90 (d, J=3.9 Hz, 1H), 7.61 (d, J=4.0 Hz, 1H), 7.37-7.31 (m, 2H), 6.98 (s, 1H), 4.10 (qd, J=9.7, 5.8 Hz, 2H). ¹⁹F{¹H} NMR (471 MHz, DMSO-d₆) δ−70.44 (s, 3F), −113.20 (s, 1F). LCMS (m/z): 420 (M+1). R^{t}: 3.060 min.

### 4-(2-(4-Fluorophenyl)-1H-pyrrolo[2,3-b]pyridin-5-yl)-N-(2,2,2-trifluoroethyl)-benzamide (10b) (Compound 15)

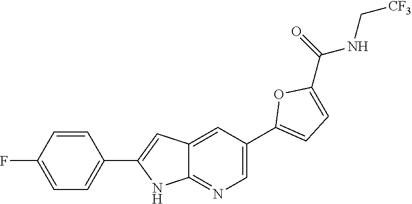 [SMILES: O=C(NCC(F)(F)F)c1ccc(-c2cnc3[nH]c(-c4ccc(F)cc4)cc3c2)o1]

Synthesized accordingly procedure, described for 7a.

Yield 32% (19 mg). ¹H NMR (500 MHz, DMSO-d₆) δ 12.42 (s, 1H), 9.14 (t, J=6.4 Hz, 1H), 8.87 (d, J=2.0 Hz, 1H), 8.48 (d, J=2.0 Hz, 1H), 8.05-8.00 (m, 2H), 7.37-7.32 (m, 2H), 7.31 (d, J=3.6 Hz, 1H), 7.16 (d, J=3.6 Hz, 1H), 7.00 (s, 1H), 4.10 (qd, J=9.7, 6.3 Hz, 2H). LCMS (m/z): 404 (M+1), Rₜ: 2.965 min.

### 5-(2-(4-Fluorophenyl)-1H-pyrrolo[2,3-b]pyridin-5-yl)-N-(2,2,2-trifluoroethyl)thiazole-2-carboxamide (10c) (Compound 21)

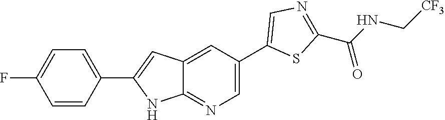 [SMILES: O=C(NCC(F)(F)F)c1ncc(-c2cnc3[nH]c(-c4ccc(F)cc4)cc3c2)s1]

Synthesized accordingly procedure, described for 7a.

Yield 50% (31 mg). ¹H NMR (500 MHz, DMSO-d₆) δ 12.36 (s, 1H), 9.57 (s, 1H), 9.02 (d, J=2.0 Hz, 1H), 8.61 (d, J=2.1 Hz, 1H), 8.51 (s, 1H), 8.05-7.99 (m, 2H), 7.37-7.31 (m, 2H), 6.99 (s, 1H), 4.12 (dt, J=13.5, 6.7 Hz, 2H). ¹⁹F{¹H} NMR (471 MHz, DMSO-d₆) δ−70.16 (s, 3F), −113.38 (s, 1F). LCMS (m/z): 421 (M+1), Rₜ: 3.112 min.

### 2-(2-(4-Fluorophenyl)-1H-pyrrolo[2,3-b]pyridin-5-yl)-N-(2,2,2-trifluoroethyl)thiazole-5-carboxamide (10d) (Compound 22)

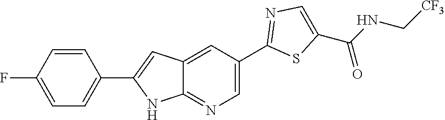 [SMILES: O=C(NCC(F)(F)F)c1cnc(-c2cnc3[nH]c(-c4ccc(F)cc4)cc3c2)s1]

Synthesized accordingly procedure, described for 7a.

Yield 2% (1 mg). ¹⁹F{¹H} NMR (471 MHz, DMSO-d₆) δ−70.38 (s, 3F), −112.94 (s, 1F).

LCMS (m/z): 421 (M+1). Rₜ: 3.020 min.

### 4-(2-(4-Fluorophenyl)-1H-pyrrolo[2,3-b]pyridin-5-yl)-N-(2,2,2-trifluoroethyl)-thiophene-2-carboxamide (10f) (Compound 1)

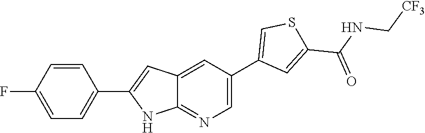 [SMILES: O=C(NCC(F)(F)F)c1cc(-c2cnc3[nH]c(-c4ccc(F)cc4)cc3c2)cs1]

Synthesized accordingly procedure, described for 7a.

Yield 36% (27 mg). ¹H NMR (500 MHz, DMSO-d₆) δ 12.32-12.23 (m, 1H), 9.14 (t, J=6.3 Hz, 1H), 8.61 (d, J=2.2 Hz, 1H), 8.44 (d, J=1.5 Hz, 1H), 8.22 (d, J=2.2 Hz, 1H), 8.19 (d, J=1.4 Hz, 1H), 8.05-7.97 (m, 2H), 7.39-7.26 (m, 2H), 6.96 (d, J=2.1 Hz, 1H), 4.13 (qd, J=9.7, 6.2 Hz, 2H). ¹⁹F NMR{¹H} (471 MHz, DMSO-d₆) δ−70.46 (s, 3F), −113.44 (s, 1F). LCMS (m/z): 420 (M+1). Rₜ: 3.114 min.

### 2-(2-(4-Fluorophenyl)-1H-pyrrolo[2,3-b]pyridin-5-yl)-N-(2,2,2-trifluoroethyl)-isonicotinamide (10e) (Compound 16)

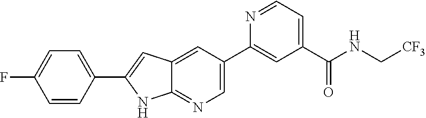 [SMILES: O=C(NCC(F)(F)F)c1ccnc(-c2cnc3[nH]c(-c4ccc(F)cc4)cc3c2)c1]

Synthesized accordingly procedure, described for 7a.

Yield 18% (11 mg). ¹H NMR (500 MHz, DMSO-d₆) δ 12.41 (s, 1H), 9.60 (t, J=6.0 Hz, 1H), 9.03 (d, J=2.1 Hz, 1H), 8.84 (dd, J=5.5, 0.6 Hz, 1H), 8.67 (d, J=2.1 Hz, 1H), 8.46 (t, J=1.2 Hz, 1H), 8.06-8.00 (m, 2H), 7.74 (dd, J=5.0, 1.5 Hz, 1H), 7.39-7.31 (m, 2H), 7.04 (s, 1H), 4.20 (qd, J=9.7, 5.6 Hz, 2H). ¹⁹F{¹H} NMR (471 MHz, DMSO-d₆) δ−70.23 (s, 3F), −113.35 (s, 1F). LCMS (m/z): 415 (M+1). Rₜ: 2.867 min.

### 5-(2-(4-Fluorophenyl)-1H-pyrrolo[2,3-b]pyridin-5-yl)-N-(2,2,2-trifluoroethyl)nicotinamide (10f) (Compound 17)

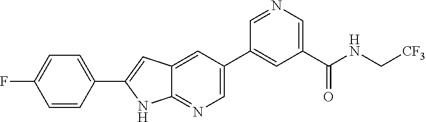 [SMILES: O=C(NCC(F)(F)F)c1cncc(-c2cnc3[nH]c(-c4ccc(F)cc4)cc3c2)c1]

Synthesized accordingly procedure, described for 7a.

Yield 10% (6 mg). ¹H NMR (500 MHz, DMSO-d₆) δ 9.48 (t, J=5.3 Hz, 1H), 9.16 (d, J=2.2 Hz, 1H), 9.02 (d, J=2.0 Hz, 1H), 8.38 (d, J=2.2 Hz, 1H), 8.11-7.98 (m, 2H), 7.42-7.28 (m, 2H), 7.02 (s, 1H), 4.19 (qd, J=9.7, 5.8 Hz, 2H). LCMS (m/z): 415 (M+1), Rₜ: 2.761 min.

### 4-(2-(4-Fluorophenyl)-1H-pyrrolo[2,3-b]pyridin-5-yl)-N-(2,2,2-trifluoroethyl)picolinamide (10g) (Compound 18)

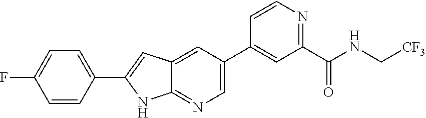 [SMILES: O=C(NCC(F)(F)F)c1cc(-c2cnc3[nH]c(-c4ccc(F)cc4)cc3c2)ccn1]

Synthesized accordingly procedure, described for 7a.

Yield 11% (7 mg). ¹H NMR (500 MHz, DMSO-d₆) δ 12.42 (s, 1H), 9.40 (t, J=6.7 Hz, 1H), 8.74 (d, J=5.1 Hz, 1H), 8.72 (d, J=2.2 Hz, 1H), 8.47 (d, J=2.2 Hz, 1H), 8.41 (d, J=1.9 Hz, 1H), 7.35 (t, J=8.8 Hz, 2H), 7.03 (s, 1H), 4.13 (qd, J=9.6, 6.6 Hz, 2H). LCMS (m/z): 415 (M+1), Rₜ: 3.105 min.

### 6-(2-(4-Fluorophenyl)-1H-pyrrolo[2,3-b]pyridin-5-yl)-N-(2,2,2-trifluoroethyl)picolinamide (10h) (Compound 19)

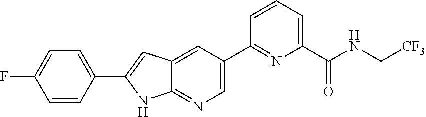 [SMILES: O=C(NCC(F)(F)F)c1cccc(-c2cnc3[nH]c(-c4ccc(F)cc4)cc3c2)n1]

Synthesized accordingly procedure, described for 7a.

Yield 8% (5 mg). ¹H NMR (500 MHz, DMSO-d₆) δ 12.41 (s, 1H), 9.51 (d, J=6.7 Hz, 1H), 9.27 (d, J=2.1 Hz, 1H), 8.85 (d, J=2.1 Hz, 1H), 8.33 (dd, J=8.0, 1.0 Hz, 1H), 8.13-7.97 (m, 4H), 7.38-7.33 (m, 2H), 7.03 (s, 1H), 4.17 (qd, J=9.7, 6.7 Hz, 2H). ¹⁹F{¹H} NMR (471 MHz, DMSO-d₆) δ−70.08 (s, 3F), −113.36 (s, 1F). LCMS (m/z): 415 (M+1). Rₜ: 3.170 min.

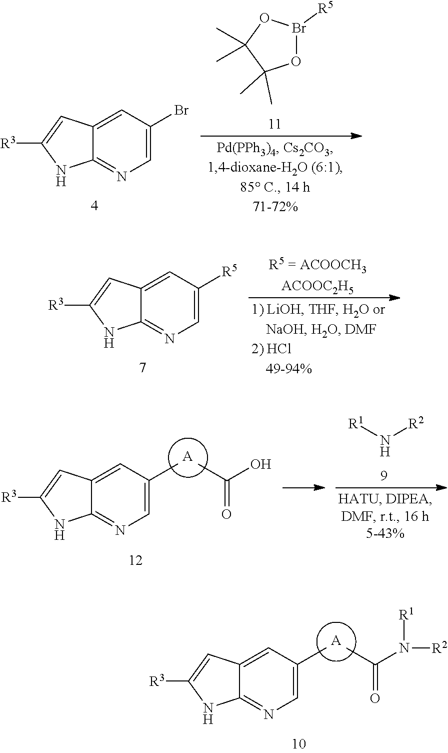

### Methyl 3-(2-(4-fluorophenyl)-1H-pyrrolo[2,3-b]pyridin-5-yl)benzoate (7d)

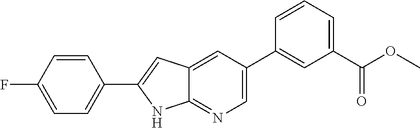 [SMILES: COC(=O)c1cccc(-c2cnc3[nH]c(-c4ccc(F)cc4)cc3c2)c1]

Synthesized accordingly procedure, described for 7a.

Yield 72% (428 mg). LCMS (m/z): 347 (M+1). Rₜ: 3.301 min.

### Methyl 4-(2-(4-fluorophenyl)-1H-pyrrolo[2,3-b]pyridin-5-yl)benzoate (7e)

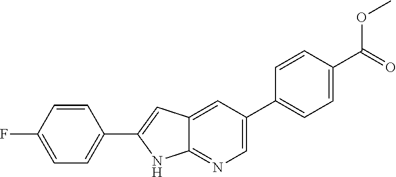 [SMILES: COC(=O)c1ccc(-c2cnc3[nH]c(-c4ccc(F)cc4)cc3c2)cc1]

Synthesized accordingly procedure, described for 7a.

Yield 71% (170 mg). LCMS (m/z): 347 (M+1). Rₜ: 3.322 min.

### 4-(2-(4-Fluorophenyl)-1H-pyrrolo[2,3-b]pyridin-5-yl)thiophene-2-carboxylic Acid (12a)

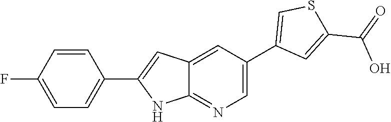 [SMILES: O=C(O)c1cc(-c2cnc3[nH]c(-c4ccc(F)cc4)cc3c2)cs1]

The methyl 4-(2-(4-fluorophenyl)-1H-pyrrolo[2,3-b]pyridin-5-yl)thiophene-2-carboxylate (7a) (1 eqv., 2.38 mmol, 840 mg), was dissolved in THF (100 ml), and aqueous solution of lithium hydroxide (8.76 eqv., 20.88 mmol, 500 mg in 100 ml of water) was added dropwise and stirred overnight. After the reaction was completed, the reaction mixture was concentrated under reduced pressure, solution was neutralized with 6N hydrochloric acid, filtered and washed with water. The compound was used in next step without further purification.

Yield 91% (733 mg). LCMS (m/z): 339 (M+1). Rₜ: 2.802 min.

### 6-(2-(4-Fluorophenyl)-1H-pyrrolo[2,3-b]pyridin-5-yl)picolinic Acid (12b)

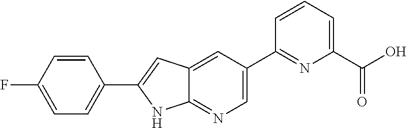 [SMILES: O=C(O)c1cccc(-c2cnc3[nH]c(-c4ccc(F)cc4)cc3c2)n1]

Synthesized accordingly procedure, described for 12a.

Yield 94% (316 mg). LCMS (m/z): 334 (M+1). Rₜ: 2.620 min.

### 4-(2-(4-Fluorophenyl)-1H-pyrrolo[2,3-b]pyridin-5-yl)thiazole-2-carboxylic Acid (12c)

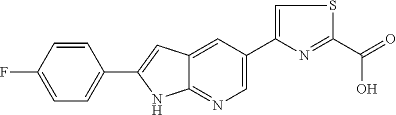 [SMILES: O=C(O)c1nc(-c2cnc3[nH]c(-c4ccc(F)cc4)cc3c2)cs1]

Synthesized accordingly procedure, described for 12a.

Yield 88% (364 mg). LCMS (m/z): 340 (M+1). Rₜ: 2.450 min.

### 3-(2-(4-Fluorophenyl)-1H-pyrrolo[2,3-b]pyridin-5-yl)benzoic Acid (12d)

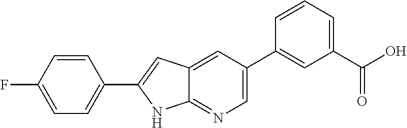 [SMILES: O=C(O)c1cccc(-c2cnc3[nH]c(-c4ccc(F)cc4)cc3c2)c1]

Synthesized accordingly procedure, described for 12a.

Yield 65% (250 mg). LCMS (m/z): 333 (M+1). Rₜ: 2.837 min.

### 4-(2-(4-Fluorophenyl)-1H-pyrrolo[2,3-b]pyridin-5-yl)benzoic Acid (12e)

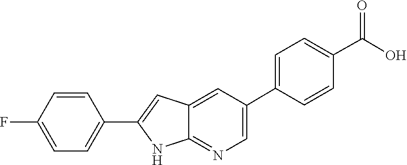 [SMILES: O=C(O)c1ccc(-c2cnc3[nH]c(-c4ccc(F)cc4)cc3c2)cc1]

The methyl 4-(2-(4-fluorophenyl)-1H-pyrrolo[2,3-b]pyridin-5-yl)benzoate (7e) (1 eqv., 433 umol, 150 mg) was dissolved in 40 ml DMF and 2 ml of 50% wt aqueous solution were added dropwise and stirred overnight. After the reaction was completed, the reaction mixture was concentrated under reduced pressure, solution was neutralized with 6N hydrochloric acid, filtered and washed with water. The compound was used in next step without further purification.

Yield 49% (70 mg). LCMS (m/z): 333 (M+1). Rₜ: 2.839 min. 4-(2-(4-Fluorophenyl)-1H-pyrrolo[2,3-b]pyridin-5-yl)-N-(3,3,3-trifluoro-2-hydroxy-propyl)thiophene-2-carboxamide (10g) (Compound 2).

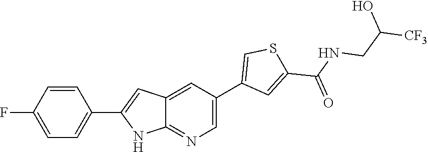 [SMILES: O=C(NCC(O)C(F)(F)F)c1cc(-c2cnc3[nH]c(-c4ccc(F)cc4)cc3c2)cs1]

Synthesized accordingly procedure, described for 6b.

Yield 5% (3 mg). ¹H NMR (500 MHz, DMSO-d₆) δ 12.29 (s, 1H), 9.06 (t, J=5.7 Hz, 1H), 8.61 (d, J=2.1 Hz, 1H), 8.43 (d, J=1.5 Hz, 1H), 8.21 (d, J=2.1 Hz, 1H), 8.14 (d, J=1.4 Hz, 1H), 8.04-7.99 (m, 2H), 7.37-7.30 (m, 2H), 6.97 (s, 1H), 4.19 (t, J=9.8 Hz, 1H), 3.64 (ddd, J=13.7, 5.8, 4.0 Hz, 1H), 3.32-3.26 (m, 2H). ¹⁹F{¹H} NMR (471 MHz, DMSO-d₆) δ−77.04 (s, 3F), −113.45 (s, 1F). LCMS (m/z): 450 (M+1). Rₜ: 2.866 min.

### (4,4-Fifluoropiperidin-1-yl)(4-(2-(4-fluorophenyl)-1H-pyrrolo[2,3-b]pyridin-5-yl)-thiophen-2-yl)methanone (10h) (Compound 3)

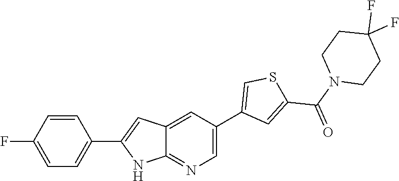 [SMILES: O=C(c1cc(-c2cnc3[nH]c(-c4ccc(F)cc4)cc3c2)cs1)N1CCC(F)(F)CC1]

Synthesized accordingly procedure, described for 6b.

Yield 6% (4 mg). ¹H NMR (500 MHz, DMSO-d₆) δ 12.28 (s, 1H), 8.66 (d, J=2.1 Hz, 1H), 8.30 (d, J=2.1 Hz, 1H), 8.13 (d, J=1.4 Hz, 1H), 8.04-7.99 (m, 2H), 7.97 (d, J=1.4 Hz, 1H), 7.36-7.30 (m, 2H), 6.95 (s, 1H), 3.79 (d, J=5.9 Hz, 4H), 2.12 (tt, J=14.3, 6.0 Hz, 4H). ¹⁹F{¹H} NMR (471 MHz, DMSO-d₆) δ−95.67 (s, 2F), −113.49 (s, 1F). LCMS (m/z): 442 (M+1). Rₜ: 3.112 min.

### 4-(2-(4-Fluorophenyl)-1H-pyrrolo[2,3-b]pyridin-5-yl)-N-(2-morpholinoethyl)-thiophene-2-carboxamide (10i) (Compound 4)

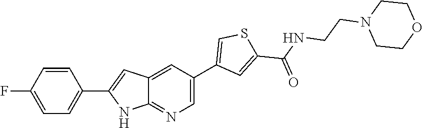 [SMILES: O=C(NCCN1CCOCC1)c1cc(-c2cnc3[nH]c(-c4ccc(F)cc4)cc3c2)cs1]

Synthesized accordingly procedure, described for 6b.

Yield 8% (5 mg). ¹H NMR (500 MHz, DMSO-d₆) δ 12.28 (s, 1H), 8.60 (d, J=2.1 Hz, 1H), 8.49 (t, J=5.7 Hz, 1H), 8.29 (d, J=1.5 Hz, 1H), 8.21 (d, J=2.2 Hz, 1H), 8.10 (d, J=1.5 Hz, 1H), 8.05-7.98 (m, 2H), 7.37-7.30 (m, 2H), 6.97 (s, 1H), 3.58 (t, J=4.6 Hz, 4H), 3.40 (q, J=7.5, 7.1 Hz, 2H), 2.49-2.46 (m, 2H), 2.43 (t, J=4.5 Hz, 4H). ¹⁹F{¹H} NMR (471 MHz, DMSO-d₆) δ−113.45 (s, 1H). LCMS (m/z): 451 (M+1). Rₜ: 2.296 min.

### N-(1-Cyclopropylethyl)-4-(2-(4-fluorophenyl)-1H-pyrrolo[2,3-b]pyridin-5-yl)-thiophene-2-carboxamide (10j) (Compound 5)

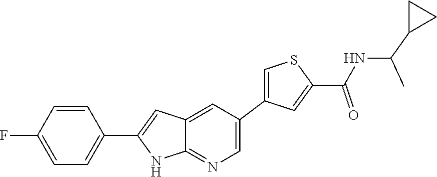 [SMILES: CC(NC(=O)c1cc(-c2cnc3[nH]c(-c4ccc(F)cc4)cc3c2)cs1)C1CC1]

Synthesized accordingly procedure, described for 6b.

Yield 40% (24 mg). ¹H NMR (500 MHz, DMSO-d₆) δ 12.29 (s, 1H), 8.62 (d, J=2.1 Hz, 1H), 8.41-8.36 (m, 2H), 8.22 (d, J=2.1 Hz, 1H), 8.10 (d, J=1.4 Hz, 1H), 8.01 (dd, J=8.8, 5.5 Hz, 2H), 7.38-7.30 (m, 2H), 6.96 (d, J=2.0 Hz, 1H), 3.45 (ddt, J=15.0, 8.3, 6.6 Hz, 1H), 1.25 (d, J=6.7 Hz, 3H), 0.99 (qt, J=8.2, 4.9 Hz, 1H), 0.52-0.46 (m, 1H), 0.44-0.38 (m, 1H), 0.36-0.30 (m, 1H), 0.26-0.20 (m, 1H). ¹⁹F{¹H} (471 MHz, DMSO-d₆) δ −113.45 (s, 1F). LCMS (m/z): 406 (M+1). Rₜ: 3160 min.

### Methyl (4-(2-(4-fluorophenyl)-1H-pyrrolo[2,3-b]pyridin-5-yl)thiophene-2-carbonyl)-D-serinate (10k) (Compound 6)

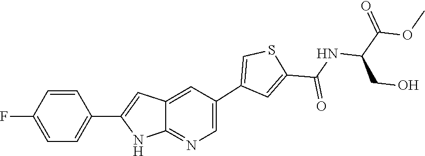 [SMILES: COC(=O)[C@@H](CO)NC(=O)c1cc(-c2cnc3[nH]c(-c4ccc(F)cc4)cc3c2)cs1]

Synthesized accordingly procedure, described for 6b.

Yield 8% (5 mg). ¹H NMR (500 MHz, DMSO-d₆) δ 12.30 (s, 1H), 9.08 (d, J=7.2 Hz, 1H), 8.65 (d, J=2.2 Hz, 1H), 8.62 (d, J=1.5 Hz, 1H), 8.26 (d, J=2.1 Hz, 1H), 8.15 (d, J=1.4 Hz, 1H), 8.05-7.99 (m, 2H), 7.36-7.30 (m, 2H), 6.96 (s, 1H), 5.74 (s, 1H), 4.51 (ddd, J=7.3, 6.0, 4.4 Hz, 1H), 3.81 (qd, J=11.2, 5.3 Hz, 2H), 3.67 (s, 3H). ¹⁹F{¹H} NMR (471 MHz, DMSO-d₆) δ−113.47 (s, 1F). LCMS (m/z): 440 (M+1). Rₜ: 2.619 min.

### Methyl (4-(2-(4-fluorophenyl)-1H-pyrrolo[2,3-b]pyridin-5-yl)thiophene-2-carbonyl)-L-serinate (101) (Compound 7)

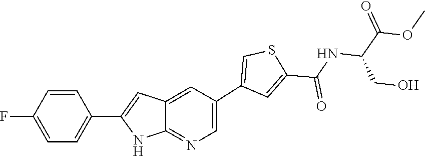 [SMILES: COC(=O)[C@H](CO)NC(=O)c1cc(-c2cnc3[nH]c(-c4ccc(F)cc4)cc3c2)cs1]

Synthesized accordingly procedure, described for 6b.

Yield 23% (15 mg). ¹H NMR (500 MHz, DMSO-d₆) δ 12.32 (s, 1H), 9.64 (d, J=6.7 Hz, 1H), 8.77 (d, J=1.5 Hz, 1H), 8.68 (d, J=2.1 Hz, 1H), 8.30 (d, J=2.1 Hz, 1H), 8.15 (d, J=1.4 Hz, 1H), 8.07-7.99 (m, 2H), 7.38-7.30 (m, 2H), 6.96 (s, 1H), 4.44 (td, J=6.5, 3.8 Hz, 1H), 3.85 (dd, J=11.5, 6.3 Hz, 2H), 3.79 (dd, J=11.5, 3.9 Hz, 1H), 3.66 (s, 3H). ¹⁹F{¹H} NMR (471 MHz, DMSO-d₆) δ−113.52 (s, 1F). LCMS (m/z): 440 (M+1). Rₜ: 2.616 min.

### N-(1,3-Dihydroxypropan-2-yl)-4-(2-(4-fluorophenyl)-1H-pyrrolo[2,3-b]pyridin-5-yl)-thiophene-2-carboxamide (10m) (Compound 8)

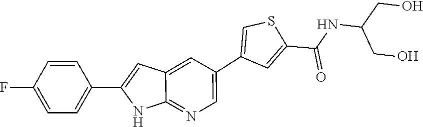 [SMILES: O=C(NC(CO)CO)c1cc(-c2cnc3[nH]c(-c4ccc(F)cc4)cc3c2)cs1]

Synthesized accordingly procedure, described for 6b.

Yield 16% (10 mg). ¹H NMR (500 MHz, DMSO-d₆) δ 12.29 (d, J=2.2 Hz, 1H), 8.63 (d, J=2.1 Hz, 1H), 8.44 (d, J=1.4 Hz, 1H), 8.23 (d, J=2.2 Hz, 1H), 8.13-8.09 (m, 2H), 8.05-7.97 (m, 2H), 7.37-7.31 (m, 2H), 6.96 (d, J=2.1 Hz, 1H), 4.74 (t, J=5.7 Hz, 2H), 3.95 (dp, J=8.3, 5.9 Hz, 1H), 3.62-3.47 (m, 4H). ¹⁹F{¹H} NMR (471 MHz, DMSO-d₆) δ −113.46 (s, 1F). LCMS (m/z): 412 (M+1). Rₜ: 2.375 min.

### N-(1-(4-(2-(4-Fluorophenyl)-1H-pyrrolo[2,3-b]pyridin-5-yl)thiophene-2-carbonyl)pyrrolidin-3-yl)methanesulfonamide (10n) (Compound 9)

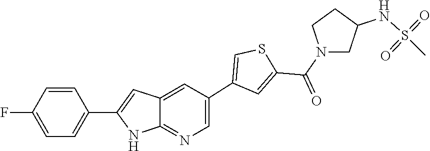 [SMILES: CS(=O)(=O)NC1CCN(C(=O)c2cc(-c3cnc4[nH]c(-c5ccc(F)cc5)cc4c3)cs2)C1]

Synthesized accordingly procedure, described for 6b.

Yield 27% (19 mg). ¹H NMR (500 MHz, DMSO-d₆) δ 12.27 (s, 1H), 8.70-8.64 (m, 1H), 8.33-8.27 (m, 1H), 8.15-8.12 (m, 1H), 8.08-7.98 (m, 3H), 7.48 (s, 1H), 7.36-7.30 (m, 2H), 6.97-6.92 (m, 1H), 4.21-3.42 (m, 5H), 3.03-2.94 (m, 3H), 2.28-1.86 (m, 2H). ¹⁹F{¹H} NMR (471 MHz, DMSO-d₆) δ−113.50 (s, 1H). LCMS (m/z): 485 (M+1). Rₜ: 2.601 min.

### (4-(2-(4-Fluorophenyl)-1H-pyrrolo[2,3-b]pyridin-5-yl)thiophen-2-yl)((1R,3r,5S)-3-hydroxy-8-azabicyclo[3.2.1]octan-8-yl)methanone (100) (Compound 10)

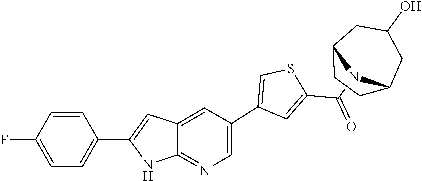 [SMILES: O=C(c1cc(-c2cnc3[nH]c(-c4ccc(F)cc4)cc3c2)cs1)N1[C@@H]2CC[C@H]1CC(O)C2]

Synthesized accordingly procedure, described for 6b.

Yield 24% (15 mg). ¹H NMR (500 MHz, DMSO-d₆) δ 12.26 (d, J=2.0 Hz, 1H), 8.66 (d, J=2.1 Hz, 1H), 8.30 (dd, J=2.2, 0.6 Hz, 1H), 8.08 (d, J=1.4 Hz, 1H), 8.04-7.99 (m, 2H), 7.92 (d, J=1.4 Hz, 1H), 7.37-7.30 (m, 2H), 6.94 (d, J=2.1 Hz, 1H), 4.70 (d, J=2.4 Hz, 1H), 4.64 (s, 2H), 4.00 (s, 1H), 2.30-2.20 (m, 2H), 2.09-1.71 (m, 7H). ¹⁹F{¹H} NMR (471 MHz, DMSO-d₆) δ−113.51 (s, 1F). LCMS (m/z): 448 (M+1). Rₜ: 2.709.

### (S)—N-(2,3-Dihydroxypropyl)-4-(2-(4-fluorophenyl)-1H-pyrrolo[2,3-b]pyridin-5-yl)-thiophene-2-carboxamide (10p) (Compound 11)

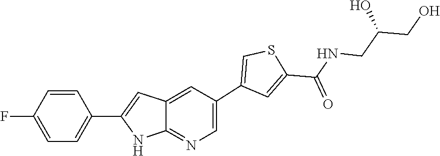 [SMILES: O=C(NC[C@H](O)CO)c1cc(-c2cnc3[nH]c(-c4ccc(F)cc4)cc3c2)cs1]

Synthesized accordingly procedure, described for 6b.

Yield 12% (7 mg). ¹H NMR (500 MHz, DMSO-d₆) δ 12.28 (s, 1H), 8.61 (d, J=2.1 Hz, 1H), 8.49 (t, J=5.8 Hz, 1H), 8.38 (d, J=1.5 Hz, 1H), 8.21 (d, J=2.2 Hz, 1H), 8.11 (d, J=1.4 Hz, 1H), 8.04-7.99 (m, 2H), 7.36-7.31 (m, 2H), 6.97 (s, 1H), 4.88 (d, J=4.9 Hz, 1H), 4.62 (t, J=5.8 Hz, 1H), 3.64 (dp, J=7.1, 5.3 Hz, 1H), 3.45-3.39 (m, 1H), 3.37 (t, J=5.6 Hz, 2H), 3.19 (ddd, J=13.1, 7.0, 5.6 Hz, 1H). ¹⁹F{¹H} NMR (471 MHz, DMSO-d₆) δ−113.45 (s, 1F). LCMS (m/z): 412 (M+1). Rₜ: 2.394 min.

### (R)-4-(2-(4-Fluorophenyl)-1H-pyrrolo[2,3-b]pyridin-5-yl)-N-(1-hydroxybutan-2-yl)-thiophene-2-carboxamide (10q) (Compound 12)

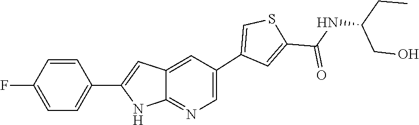 [SMILES: CC[C@H](CO)NC(=O)c1cc(-c2cnc3[nH]c(-c4ccc(F)cc4)cc3c2)cs1]

Synthesized accordingly procedure, described for 6b.

Yield 20% (12 mg). ¹⁹F{¹H} NMR (471 MHz, DMSO-d₆) δ−113.46 (s, 1F). LCMS (m/z): 410 (M+1). Rₜ: 2.694 min.

### 3-(2-(4-Fluorophenyl)-1H-pyrrolo[2,3-b]pyridin-5-yl)-N-(2,2,2-trifluoroethyl)-benzamide (10r) (Compound 13)

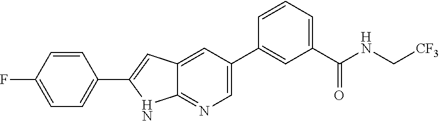 [SMILES: O=C(NCC(F)(F)F)c1cccc(-c2cnc3[nH]c(-c4ccc(F)cc4)cc3c2)c1]

Synthesized accordingly procedure, described for 6b.

Yield 11% (7 mg). ¹H NMR (500 MHz, DMSO-d₆) δ 12.33 (s, 1H), 9.28 (t, J=6.3 Hz, 1H), 8.63 (d, J=2.2 Hz, 1H), 8.30 (d, J=2.1 Hz, 1H), 8.27 (t, J=1.8 Hz, 1H), 8.07-8.01 (m, 2H), 7.99-7.95 (m, 1H), 7.89 (dt, J=7.8, 1.4 Hz, 1H), 7.63 (t, J=7.7 Hz, 1H), 7.38-7.33 (m, 2H), 7.01 (d, J=1.3 Hz, 1H), 4.17 (qd, J=9.7, 6.2 Hz, 2H). ¹⁹F{¹H} NMR (471 MHz, DMSO-d₆) δ−70.35 (s, 3F), −113.44 (s, 1F). LCMS (m/z): 414 (M+1). Rₜ: 3.050 min.

### 4-(2-(4-Fluorophenyl)-1H-pyrrolo[2,3-b]pyridin-5-yl)-N-(2,2,2-trifluoroethyl)-benzamide (10s) (Compound 14)

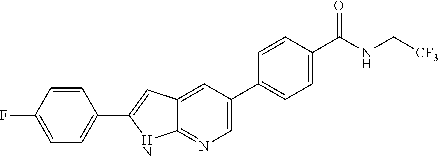 [SMILES: O=C(NCC(F)(F)F)c1ccc(-c2cnc3[nH]c(-c4ccc(F)cc4)cc3c2)cc1]

Synthesized accordingly procedure, described for 6b.

Yield 11% (7 mg). ¹H NMR (500 MHz, DMSO-d₆) δ 12.31 (s, 1H), 9.16 (t, J=6.3 Hz, 1H), 8.61 (d, J=2.2 Hz, 1H), 8.29 (d, J=2.2 Hz, 1H), 8.02 (ddd, J=8.5, 6.1, 2.0 Hz, 4H), 7.93-7.86 (m, 2H), 7.38-7.30 (m, 2H), 6.99 (s, 1H), 4.12 (tt, J=9.8, 4.9 Hz, 2H). LCMS (m/z): 414 (M+1), Rₜ: 3.028 min.

### 2-(4-(2-(4-Fluorophenyl)-1H-pyrrolo[2,3-b]pyridin-5-yl)thiophene-2-carboxamido)-ethane-1-sulfonic Acid (10t) (Compound 23)

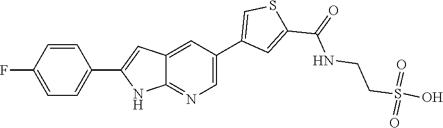 [SMILES: O=C(NCCS(=O)(=O)O)c1cc(-c2cnc3[nH]c(-c4ccc(F)cc4)cc3c2)cs1]

Synthesized accordingly procedure, described for 6b.

Yield 40% (26 mg). ¹H NMR (500 MHz, DMSO-d₆) δ 8.59 (d, J=2.1 Hz, 1H), 8.54 (t, J=5.5 Hz, 1H), 8.20 (d, J=2.2 Hz, 1H), 8.15 (d, J=1.5 Hz, 1H), 8.08 (d, J=1.5 Hz, 1H), 8.03-7.98 (m, 2H), 7.36-7.29 (m, 2H), 6.96 (s, 1H), 3.56-3.48 (m, 2H), 3.16 (s, 1H), 2.72-2.65 (m, 2H). LCMS (m/z): 446 (M+1), Rₜ: 2.239 min.

### 2,2,2-Frifluoro-N-(1-(4-(2-(4-fluorophenyl)-1H-pyrrolo[2,3-b]pyridin-5-yl)thiophene-2-carbonyl)pyrrolidin-3-yl)acetamide (10u) (Compound 24)

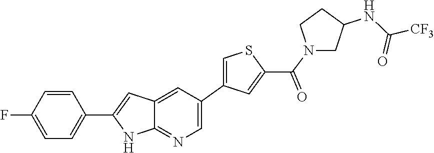 [SMILES: O=C(c1cc(-c2cnc3[nH]c(-c4ccc(F)cc4)cc3c2)cs1)N1CCC(NC(=O)C(F)(F)F)C1]

Synthesized accordingly procedure, described for 6b.

Yield 5% (4 mg). ¹H NMR (500 MHz, DMSO-d₆) δ 12.26 (s, 1H), 9.75 (s, 1H), 8.67 (d, J=2.1 Hz, 1H), 8.31 (s, 1H), 8.06-7.98 (m, 2H), 7.38-7.29 (m, 2H), 6.95 (s, 1H), 4.47 (d, J=30.6 Hz, 1H), 4.23 (t, J=8.7 Hz, OH), 4.03 (d, J=8.0 Hz, 1H), 3.91-3.75 (m, 1H), 3.71-3.55 (m, 1H), 2.34-1.93 (m, 2H).). LCMS (m/z): 503 (M+1), Rₜ: 2.901 min

### (R)—N-(2,3-Dihydroxypropyl)-4-(2-(4-fluorophenyl)-1H-pyrrolo[2,3-b]pyridin-5-yl)thiophene-2-carboxamide (10v) (Compound 25)

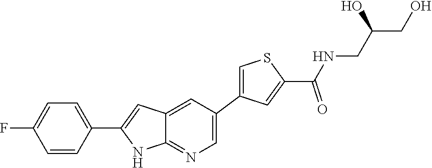 [SMILES: O=C(NC[C@@H](O)CO)c1cc(-c2cnc3[nH]c(-c4ccc(F)cc4)cc3c2)cs1]

Synthesized accordingly procedure, described for 6b.

Yield 38% (23 mg). ¹H NMR (500 MHz, DMSO-d₆) δ 12.26 (s, 1H), 8.61 (d, J=2.1 Hz, 1H), 8.46 (t, J=5.8 Hz, 1H), 8.37 (d, J=1.5 Hz, 1H), 8.21 (d, J=2.1 Hz, 1H), 8.10 (d, J=1.5 Hz, 1H), 8.05-7.97 (m, 2H), 7.38-7.29 (m, 2H), 6.96 (d, J=1.9 Hz, 1H), 4.85 (d, J=5.0 Hz, 1H), 4.59 (t, J=5.8 Hz, 1H), 3.65 (dp, J=6.9, 5.3 Hz, 1H), 3.46-3.34 (m, 3H), 3.24-3.15 (m, 1H). LCMS (m/z): 412 (M+1), Rₜ: 2.400 min.

### (S)-4-(2-(4-Fluorophenyl)-1H-pyrrolo[2,3-b]pyridin-5-yl)-N-(1-hydroxybutan-2-yl)thiophene-2-carboxamide (10w) (Compound 26)

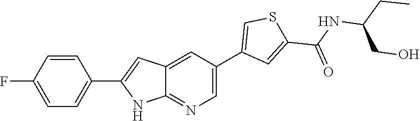 [SMILES: CC[C@@H](CO)NC(=O)c1cc(-c2cnc3[nH]c(-c4ccc(F)cc4)cc3c2)cs1]

Synthesized accordingly procedure, described for 6b.

Yield 18% (11 mg). ¹H NMR (500 MHz, DMSO-d₆) δ 12.27 (s, 1H), 8.63 (d, J=2.1 Hz, 1H), 8.42 (d, J=1.5 Hz, 1H), 8.23 (d, J=2.1 Hz, 1H), 8.12-8.06 (m, 2H), 8.04-7.99 (m, 2H), 7.39-7.29 (m, 2H), 6.97 (d, J=1.6 Hz, 1H), 4.74 (t, J=5.7 Hz, 1H), 3.92-3.80 (m, 1H), 3.49 (dt, J=10.5, 5.1 Hz, 1H), 3.43 (q, J=5.4 Hz, 1H), 1.69 (dtd, J=14.9, 7.4, 4.9 Hz, 1H), 1.48 (ddd, J=13.8, 9.0, 7.3 Hz, 1H), 0.92 (t, J=7.4 Hz, 3H). LCMS (m/z): 410 (M+1), Rₜ: 2.703 min.

### 4-(2-(4-Fluorophenyl)-1H-pyrrolo[2,3-b]pyridin-5-yl)-N-(3-(pyrrolidin-1-yl)propyl)-thiophene-2-carboxamide (10x) (Compound 28)

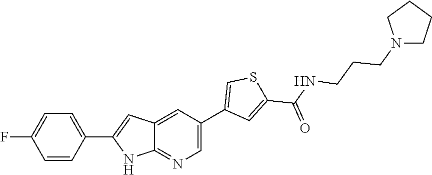 [SMILES: O=C(NCCCN1CCCC1)c1cc(-c2cnc3[nH]c(-c4ccc(F)cc4)cc3c2)cs1]

Synthesized accordingly procedure, described for 6b.

Yield 23% (15 mg). ¹H NMR (500 MHz, DMSO-d₆) δ 8.60 (d, J=2.1 Hz, 1H), 8.55 (t, J=5.5 Hz, 1H), 8.27 (d, J=1.5 Hz, 1H), 8.20 (d, J=2.1 Hz, 1H), 8.08 (d, J=1.4 Hz, 1H), 8.04-7.98 (m, 2H), 7.36-7.31 (m, 2H), 6.96 (s, 1H), 3.31 (q, J=6.7 Hz, 4H), 2.47-2.42 (m, 6H), 1.71-1.67 (m, 4H). LCMS (m/z): 449 (M+1), Rₜ: 2.271 min.

### (4-(2-(4-Fluorophenyl)-1H-pyrrolo[2,3-b]pyridin-5-yl)thiophen-2-yl)(4-(methyl-sulfonyl)piperazin-1-yl)methanone (10y) (Compound 29)

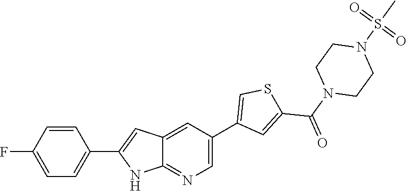 [SMILES: CS(=O)(=O)N1CCN(C(=O)c2cc(-c3cnc4[nH]c(-c5ccc(F)cc5)cc4c3)cs2)CC1]

Synthesized accordingly procedure, described for 6b.

Yield 1% (1 mg). ¹H NMR (500 MHz, DMSO-d₆) δ 12.27 (s, 1H), 8.66 (d, J=2.2 Hz, 1H), 8.29 (d, J=2.1 Hz, 1H), 8.13 (d, J=1.4 Hz, 1H), 8.06-7.98 (m, 2H), 7.93 (d, J=1.4 Hz, 1H), 7.37-7.30 (m, 2H), 6.95 (s, 1H), 3.82 (s, 4H), 3.24 (t, J=5.2 Hz, 4H), 2.93 (s, 3H). LCMS (m/z): 485 (M+1), Rₜ: 2.755 min.

### 4-(2-(4-Fluorophenyl)-1H-pyrrolo[2,3-b]pyridin-5-yl)-N-(1,1,1-trifluoro-3-methylbutan-2-yl)thiophene-2-carboxamide (10z) (Compound 30)

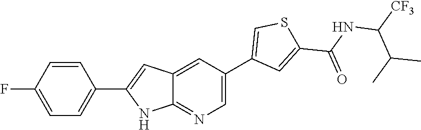 [SMILES: CC(C)C(NC(=O)c1cc(-c2cnc3[nH]c(-c4ccc(F)cc4)cc3c2)cs1)C(F)(F)F]

Synthesized accordingly procedure, described for 6b.

Yield 26% (18 mg). ¹H NMR (500 MHz, DMSO-d₆) δ 12.33-12.25 (m, 1H), 8.69 (d, J=9.6 Hz, 1H), 8.64 (d, J=2.1 Hz, 1H), 8.59 (d, J=1.5 Hz, 1H), 8.25 (d, J=2.2 Hz, 1H), 8.20 (d, J=1.4 Hz, 1H), 8.04-7.98 (m, 2H), 7.38-7.30 (m, 2H), 6.97 (d, J=1.7 Hz, 1H), 4.64-4.54 (m, 1H), 2.26-2.15 (m, J=6.8 Hz, 1H), 1.04 (d, J=6.8 Hz, 7H). LCMS (m/z): 462 (M+1), Rₜ: 3.409 min.

### (R)—N-(2,3-Dihydroxypropyl)-3-(2-(4-fluorophenyl)-1H-pyrrolo[2,3-b]pyridin-5-yl)benzamide (Compound 48)

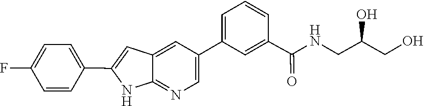 [SMILES: O=C(NC[C@@H](O)CO)c1cccc(-c2cnc3[nH]c(-c4ccc(F)cc4)cc3c2)c1]

Synthesized accordingly procedure, described for 6b.

Yield 37% (18 mg). ¹H NMR (500 MHz, DMSO-d₆) δ 12.28 (s, 1H), 8.61 (d, J=2.2 Hz, 1H), 8.59 (t, J=5.8 Hz, 1H), 8.28 (d, J=2.2 Hz, 1H), 8.23 (t, J=1.8 Hz, 1H), 8.05-8.00 (m, 2H), 7.90 (ddd, J=7.7, 1.9, 1.1 Hz, 1H), 7.84 (dt, J=7.9, 1.3 Hz, 1H), 7.57 (t, J=7.7 Hz, 1H), 7.37-7.29 (m, 2H), 6.99 (d, J=1.3 Hz, 1H), 4.84 (d, J=4.9 Hz, 1H), 4.58 (t, J=5.8 Hz, 1H), 3.67 (dq, J=6.8, 5.2 Hz, 1H), 3.44 (dt, J=13.3, 5.5 Hz, 1H), 3.38 (t, J=5.7 Hz, 2H), 3.24 (ddd, J=13.0, 7.0, 5.7 Hz, 1H). ¹⁹F{¹H} NMR (471 MHz, DMSO-d₆) δ −113.49 (s, 1F). LCMS (m/z): 406 (M+1). Rₜ: 2.422 min.

### N-(1,3-Dihydroxypropan-2-yl)-3-(2-(4-fluorophenyl)-1H-pyrrolo[2,3-b]pyridin-5-yl)benzamide (10ab) (Compound 49)

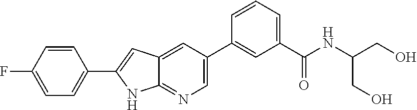 [SMILES: O=C(NC(CO)CO)c1cccc(-c2cnc3[nH]c(-c4ccc(F)cc4)cc3c2)c1]

Synthesized accordingly procedure, described for 6b.

Yield 39% (19 mg). ¹H NMR (500 MHz, DMSO-d₆) δ 12.32-12.22 (m, 1H), 8.62 (d, J=2.1 Hz, 1H), 8.28 (d, J=2.1 Hz, 1H), 8.23 (t, J=1.9 Hz, 1H), 8.16 (d, J=8.1 Hz, 1H), 8.05-7.99 (m, 2H), 7.89 (ddd, J=7.7, 1.8, 1.1 Hz, 1H), 7.85 (dt, J=7.9, 1.3 Hz, 1H), 7.57 (t, J=7.7 Hz, 1H), 7.40-7.30 (m, 2H), 6.99 (d, J=1.9 Hz, 1H), 4.68 (t, J=5.8 Hz, 2H), 4.02 (dp, J=8.0, 5.9 Hz, 1H), 3.56 (td, J=5.8, 1.8 Hz, 4H). ¹⁹F{¹H} NMR (471 MHz, DMSO-d₆) δ−113.49 (s, 1F). LCMS (m/z): 406 (M+1). Rₜ: 2.394 min.

### 3-(2-(4-Fluorophenyl)-1H-pyrrolo[2,3-b]pyridin-5-yl)-N-(3,3,3-trifluoro-2-hydroxy-propyl)benzamide (10ac) (Compound 50)

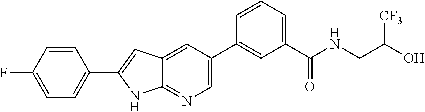 [SMILES: O=C(NCC(O)C(F)(F)F)c1cccc(-c2cnc3[nH]c(-c4ccc(F)cc4)cc3c2)c1]

Synthesized accordingly procedure, described for 6b.

Yield 26% (14 mg). ¹H NMR (500 MHz, DMSO-d₆) δ 12.27 (s, 1H), 8.62 (d, J=2.2 Hz, 1H), 8.28 (d, J=2.2 Hz, 1H), 8.25 (t, J=1.8 Hz, 1H), 8.06-7.99 (m, 2H), 7.95-7.90 (m, 1H), 7.85 (dt, J=7.8, 1.3 Hz, 1H), 7.59 (t, J=7.7 Hz, 1H), 7.36-7.31 (m, 2H), 6.99 (s, 1H), 4.22 (qd, J=7.6, 4.2 Hz, 1H), 3.67 (ddd, J=13.6, 5.7, 4.3 Hz, 1H), 3.39-3.33 (m, 2H). ¹⁹F{¹H} NMR (471 MHz, DMSO-d₆) δ−76.99 (s, 3F), −113.47 (s, 1F). LCMS (m/z): 444 (M+1). Rₜ: 2.887 min.

### (S)—N-(2,3-Dihydroxypropyl)-3-(2-(4-fluorophenyl)-1H-pyrrolo[2,3-b]pyridin-5-yl)-benzamide (10ad) (Compound 51)

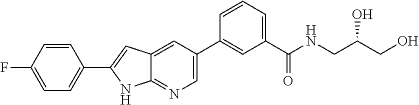 [SMILES: O=C(NC[C@H](O)CO)c1cccc(-c2cnc3[nH]c(-c4ccc(F)cc4)cc3c2)c1]

Synthesized accordingly procedure, described for 6b.

Yield 39% (19 mg). ¹H NMR (500 MHz, DMSO-d₆) δ 12.28 (s, 1H), 8.63-8.56 (m, 2H), 8.28 (d, J=2.2 Hz, 1H), 8.23 (t, J=1.8 Hz, 1H), 8.06-7.98 (m, 2H), 7.90 (ddd, J=7.7, 1.9, 1.1 Hz, 1H), 7.84 (dt, J=7.9, 1.2 Hz, 1H), 7.57 (t, J=7.7 Hz, 1H), 7.37-7.29 (m, 2H), 6.99 (d, J=1.7 Hz, 1H), 4.85 (d, J=4.9 Hz, 1H), 4.59 (t, J=5.8 Hz, 1H), 3.68 (dt, J=6.6, 4.9 Hz, 1H), 3.44 (dt, J=13.3, 5.5 Hz, 1H), 3.38 (t, J=5.6 Hz, 2H), 3.24 (ddd, J=13.0, 6.9, 5.6 Hz, 1H). ¹⁹F{¹H} NMR (471 MHz, DMSO-d₆) δ−113.49 (s, 1F). LCMS (m/z): 406 (M+1). Rₜ: 2.419 min.

### (R)—N-(2,3-Dihydroxypropyl)-6-(2-(4-fluorophenyl)-1H-pyrrolo[2,3-b]pyridin-5-yl)-picolinamide (10ae) (Compound 52)

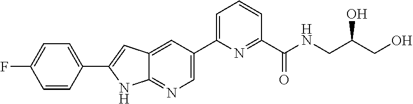 [SMILES: O=C(NC[C@@H](O)CO)c1cccc(-c2cnc3[nH]c(-c4ccc(F)cc4)cc3c2)n1]

Synthesized accordingly procedure, described for 6b.

Yield 43% (26 mg). ¹H NMR (500 MHz, DMSO-d₆) δ 12.36 (s, 1H), 9.16 (d, J=2.2 Hz, 1H), 8.95 (t, J=6.0 Hz, 1H), 8.78 (d, J=2.1 Hz, 1H), 8.27 (dd, J=8.0, 1.0 Hz, 1H), 8.11-8.01 (m, 3H), 7.98 (dd, J=7.6, 0.9 Hz, 1H), 7.38-7.31 (m, 2H), 7.02 (d, J=1.7 Hz, 1H), 4.97 (d, J=5.0 Hz, 1H), 4.71 (t, J=5.7 Hz, 1H), 3.71 (dp, J=6.7, 5.2 Hz, 1H), 3.56 (ddd, J=13.3, 6.4, 5.1 Hz, 1H), 3.47-3.32 (m, 3H). ¹⁹F{¹H} NMR (471 MHz, DMSO-d₆) δ −113.38 (s, 1F). LCMS (m/z): 407 (M+1). Rₜ: 2.426 min.

### (R)-3-(2-(4-Fluorophenyl)-1H-pyrrolo[2,3-b]pyridin-5-yl)-N-(1-hydroxybutan-2-yl)benzamide (10af) (Compound 53)

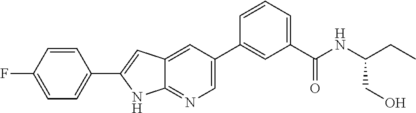 [SMILES: CC[C@H](CO)NC(=O)c1cccc(-c2cnc3[nH]c(-c4ccc(F)cc4)cc3c2)c1]

Synthesized accordingly procedure, described for 6b.

Yield 12% (6 mg). ¹H NMR (500 MHz, DMSO-d₆) δ 12.28 (s, 1H), 8.62 (d, J=2.2 Hz, 1H), 8.27 (d, J=2.2 Hz, 1H), 8.21 (q, J=1.7 Hz, 1H), 8.17 (d, J=8.5 Hz, 1H), 8.05-8.00 (m, 2H), 7.91-7.82 (m, 2H), 7.57 (t, J=7.7 Hz, 1H), 7.39-7.31 (m, 2H), 6.99 (d, J=1.3 Hz, 1H), 4.70 (t, J=5.7 Hz, 1H), 3.97-3.87 (m, 1H), 3.50 (dt, J=11.1, 5.6 Hz, 1H), 3.43 (dt, J=10.7, 5.9 Hz, 1H), 1.75-1.64 (m, 1H), 1.54-1.44 (m, 1H), 0.90 (t, J=7.4 Hz, 3H). ¹⁹F{¹H} NMR (471 MHz, DMSO-d₆) δ−113.50 (s, 1F). LCMS (m/z): 404 (M+1). Rₜ: 2.718 min.

### (S)-3-(2-(4-Fluorophenyl)-1H-pyrrolo[2,3-b]pyridin-5-yl)-N-(1-hydroxybutan-2-yl)benzamide (10ag) (Compound 54)

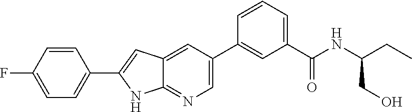 [SMILES: CC[C@@H](CO)NC(=O)c1cccc(-c2cnc3[nH]c(-c4ccc(F)cc4)cc3c2)c1]

Synthesized accordingly procedure, described for 6b.

Yield 8% (4 mg). ¹H NMR (500 MHz, DMSO-d₆) δ 12.28 (s, 1H), 8.62 (d, J=2.2 Hz, 1H), 8.27 (d, J=2.2 Hz, 1H), 8.21 (t, J=1.8 Hz, 1H), 8.17 (d, J=8.5 Hz, 1H), 8.06-8.00 (m, 2H), 7.92-7.84 (m, 2H), 7.57 (t, J=7.7 Hz, 1H), 7.39-7.29 (m, 2H), 6.99 (d, J=1.7 Hz, 1H), 4.70 (t, J=5.8 Hz, 1H), 3.92 (td, J=8.6, 5.1 Hz, 1H), 3.50 (dt, J=11.0, 5.6 Hz, 1H), 3.43 (dt, J=11.2, 6.0 Hz, 1H), 1.76-1.65 (m, 1H), 1.49 (ddt, J=16.3, 14.5, 7.4 Hz, 1H), 0.90 (t, J=7.4 Hz, 3H). ¹⁹F{¹H} NMR (471 MHz, DMSO-d₆) δ−113.50. LCMS (m/z): 404 (M+1). Rₜ: 2.714 min.

### N-(1,3-Dihydroxypropan-2-yl)-6-(2-(4-fluorophenyl)-1H-pyrrolo[2,3-b]pyridin-5-yl)picolinamide (10ah) (Compound 55)

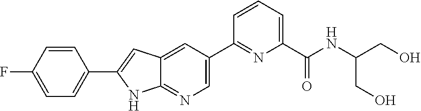 [SMILES: O=C(NC(CO)CO)c1cccc(-c2cnc3[nH]c(-c4ccc(F)cc4)cc3c2)n1]

Synthesized accordingly procedure, described for 6b.

Yield 34% (21 mg). ¹H NMR (500 MHz, DMSO-d₆) δ 12.36 (s, 1H), 9.10 (d, J=2.1 Hz, 1H), 8.71 (d, J=2.1 Hz, 1H), 8.54 (d, J=8.7 Hz, 1H), 8.26 (dd, J=8.0, 1.0 Hz, 1H), 8.10-7.96 (m, 4H), 7.38-7.30 (m, 2H), 7.04 (s, 1H), 4.89 (t, J=5.5 Hz, 2H), 4.01 (dp, J=8.6, 5.5 Hz, 1H), 3.62 (dq, J=25.9, 6.0 Hz, 4H). ¹⁹F{¹H} NMR (471 MHz, DMSO-d₆) δ −113.36 (s, 1F). LCMS (m/z): 407 (M+1). Rₜ: 2.412 min.

### (R)-6-(2-(4-Fluorophenyl)-1H-pyrrolo[2,3-b]pyridin-5-yl)-N-(1-hydroxybutan-2-yl)-picolinamide (10ai) (Compound 56)

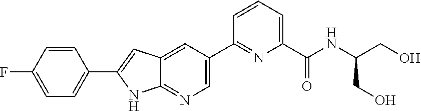

Synthesized accordingly procedure, described for 6b.

Yield 18% (11 mg). ¹H NMR (500 MHz, DMSO-d₆) δ 12.35 (s, 1H), 9.13 (d, J=2.1 Hz, 1H), 8.74 (d, J=2.1 Hz, 1H), 8.49 (d, J=9.1 Hz, 1H), 8.25 (dd, J=7.9, 1.1 Hz, 1H), 8.10-8.01 (m, 3H), 7.98 (dd, J=7.6, 0.9 Hz, 1H), 7.38-7.30 (m, 2H), 7.04 (s, 1H), 4.86 (t, J=5.5 Hz, 1H), 3.94 (tdd, J=10.7, 7.0, 4.4 Hz, 1H), 3.59 (dt, J=10.6, 5.2 Hz, 1H), 3.53 (dt, J=11.0, 5.4 Hz, 1H), 1.71 (dtd, J=14.9, 7.4, 5.6 Hz, 1H), 1.66-1.55 (m, 1H), 0.92 (t, J=7.4 Hz, 3H). ¹⁹F{¹H} NMR (471 MHz, DMSO-d₆) δ−113.39 (s, 1F). LCMS (m/z): 405 (M+1). Rₜ: 2.754 min.

### 6-(2-(4-Fluorophenyl)-1H-pyrrolo[2,3-b]pyridin-5-yl)-N-(3,3,3-trifluoro-2-hydroxy-propyl)picolinamide (10aj) (Compound 57)

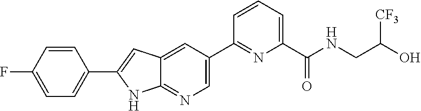 [SMILES: O=C(NCC(O)C(F)(F)F)c1cccc(-c2cnc3[nH]c(-c4ccc(F)cc4)cc3c2)n1]

Synthesized accordingly procedure, described for 6b.

Yield 38% (25 mg). ¹H NMR (500 MHz, DMSO-d₆) δ 12.37 (s, 1H), 9.22 (d, J=2.1 Hz, 1H), 9.14 (t, J=6.1 Hz, 1H), 8.82 (d, J=2.2 Hz, 1H), 8.29 (dd, J=8.0, 1.0 Hz, 1H), 8.10-8.00 (m, 3H), 7.98 (dd, J=7.6, 0.9 Hz, 1H), 7.38-7.31 (m, 2H), 7.02 (s, 1H), 6.57 (s, 1H), 4.33 (d, J=6.8 Hz, 1H), 3.70 (ddd, J=13.6, 5.9, 4.2 Hz, 1H), 3.54 (ddd, J=13.6, 8.4, 6.3 Hz, 1H). ¹⁹F{¹H} NMR (471 MHz, DMSO-d₆) δ−77.03 (s, 3F), −113.38 (s, 1F). LCMS (m/z): 445 (M+1). Rₜ: 2.924 min.

### (S)—N-(2,3-Dihydroxypropyl)-6-(2-(4-fluorophenyl)-1H-pyrrolo[2,3-b]pyridin-5-yl)-picolinamide (10ak) (Compound 58)

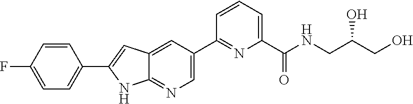 [SMILES: O=C(NC[C@H](O)CO)c1cccc(-c2cnc3[nH]c(-c4ccc(F)cc4)cc3c2)n1]

Synthesized accordingly procedure, described for 6b.

Yield 56% (34 mg). ¹H NMR (500 MHz, DMSO-d₆) δ 12.36 (s, 1H), 9.16 (d, J=2.2 Hz, 1H), 8.95 (t, J=6.0 Hz, 1H), 8.78 (d, J=2.1 Hz, 1H), 8.26 (dd, J=8.0, 1.0 Hz, 1H), 8.09-8.01 (m, 3H), 7.98 (dd, J=7.6, 1.0 Hz, 1H), 7.39-7.29 (m, 2H), 7.02 (s, 1H), 4.98 (d, J=5.0 Hz, 1H), 4.72 (t, J=5.7 Hz, 1H), 3.75-3.67 (m, 1H), 3.56 (ddd, J=13.4, 6.4, 5.1 Hz, 1H), 3.47-3.33 (m, 3H). ¹⁹F{¹H} NMR (471 MHz, DMSO-d₆) δ−113.38 (s, 1F). LCMS (m/z): 407 (M+1). Rₜ: 2.430 min.

### (S)-6-(2-(4-Fluorophenyl)-1H-pyrrolo[2,3-b]pyridin-5-yl)-N-(1-hydroxybutan-2-yl)-picolinamide (10al) (Compound 59)

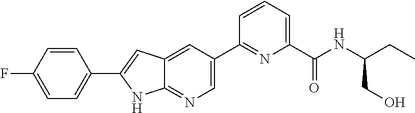 [SMILES: CC[C@@H](CO)NC(=O)c1cccc(-c2cnc3[nH]c(-c4ccc(F)cc4)cc3c2)n1]

Synthesized accordingly procedure, described for 6b.

Yield 31% (19 mg). ¹H NMR (500 MHz, DMSO-d₆) δ 12.36 (s, 1H), 9.13 (d, J=2.1 Hz, 1H), 8.74 (d, J=2.1 Hz, 1H), 8.49 (d, J=9.1 Hz, 1H), 8.25 (dd, J=8.0, 1.0 Hz, 1H), 8.10-8.01 (m, 3H), 7.98 (dd, J=7.6, 1.0 Hz, 1H), 7.38-7.31 (m, 2H), 7.04 (s, 1H), 4.86 (t, J=5.6 Hz, 1H), 3.94 (ddt, J=14.2, 8.7, 5.2 Hz, 1H), 3.56 (ddt, J=33.3, 10.2, 4.2 Hz, 2H), 1.77-1.55 (m, 2H), 0.92 (t, J=7.4 Hz, 3H). ¹⁹F{¹H} NMR (471 MHz, DMSO-d₆) δ−113.39 (s, 1F). LCMS (m/z): 405 (M+1). Rₜ: 2.753 min.

### 4-(2-(4-Fluorophenyl)-1H-pyrrolo[2,3-b]pyridin-5-yl)-N-(3,3,3-trifluoro-2-hydroxy-propyl)thiazole-2-carboxamide (10am) (Compound 65)

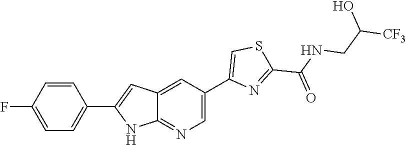 [SMILES: O=C(NCC(O)C(F)(F)F)c1nc(-c2cnc3[nH]c(-c4ccc(F)cc4)cc3c2)cs1]

Synthesized accordingly procedure, described for 6b.

Yield 6% (4 mg). ¹H NMR (500 MHz, DMSO-d₆) δ 12.35 (s, 1H), 9.08 (t, J=6.0 Hz, 1H), 9.00 (d, J=2.1 Hz, 1H), 8.59 (d, J=2.0 Hz, 1H), 8.46 (s, 1H), 8.07-7.99 (m, 2H), 7.39-7.29 (m, 2H), 6.99 (s, 1H), 6.62 (s, 1H), 4.31 (s, 1H), 3.62 (ddd, J=13.6, 5.6, 4.2 Hz, 1H), 3.48 (ddd, J=14.1, 8.3, 6.4 Hz, 1H). ¹⁹F{¹H} NMR (471 MHz, DMSO-d₆) δ−77.05 (s, 3F), −113.39 (s, 1F). LCMS (m/z): 451 (M+1). Rₜ: 2.919 min.

### (S)—N-(2,3-Dihydroxypropyl)-4-(2-(4-fluorophenyl)-1H-pyrrolo[2,3-b]pyridin-5-yl)-thiazole-2-carboxamide (10an) (Compound 60)

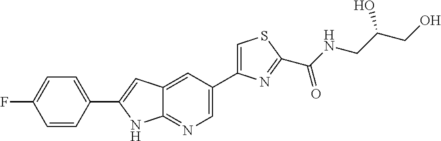 [SMILES: O=C(NC[C@H](O)CO)c1nc(-c2cnc3[nH]c(-c4ccc(F)cc4)cc3c2)cs1]

Synthesized accordingly procedure, described for 6b.

Yield 15% (9 mg). ¹H NMR (500 MHz, DMSO-d₆) δ 12.34 (s, 1H), 8.98 (d, J=2.1 Hz, 1H), 8.72 (t, J=6.0 Hz, 1H), 8.58 (d, J=2.0 Hz, 1H), 8.43 (s, 1H), 8.06-7.98 (m, 2H), 7.38-7.29 (m, 2H), 6.99 (s, 1H), 4.94 (d, J=5.0 Hz, 1H), 4.67 (t, J=5.7 Hz, 1H), 3.70 (t, J=5.9 Hz, 1H), 3.52-3.21 (m, 4H). ¹⁹F{¹H} NMR (471 MHz, DMSO-d₆) δ−113.41 (s, 1F). LCMS (m/z): 413 (M+1). Rₜ: 2.410 min.

### (S)-4-(2-(4-Fluorophenyl)-1H-pyrrolo[2,3-b]pyridin-5-yl)-N-(1-hydroxybutan-2-yl)-thiazole-2-carboxamide (10ao) (Compound 61)

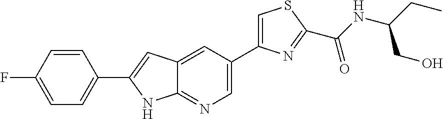 [SMILES: CC[C@@H](CO)NC(=O)c1nc(-c2cnc3[nH]c(-c4ccc(F)cc4)cc3c2)cs1]

Synthesized accordingly procedure, described for 6b.

Yield 25% (15 mg). ¹H NMR (500 MHz, DMSO-d6) δ 12.35 (s, 1H), 9.01 (d, J=2.0 Hz, 1H), 8.60 (d, J=2.1 Hz, 1H), 8.45-8.41 (m, 2H), 8.02 (ddd, J=11.0, 5.5, 2.8 Hz, 2H), 7.37-7.30 (m, 2H), 6.99 (d, J=1.5 Hz, 1H), 4.82 (t, J=5.7 Hz, 1H), 3.89 (tq, J=9.0, 5.6 Hz, 1H), 3.52 (dh, J=22.1, 5.7 Hz, 2H), 1.69 (dtd, J=14.8, 7.4, 5.1 Hz, 1H), 1.62-1.49 (m, 1H), 0.91 (t, J=7.4 Hz, 3H). ¹⁹F{¹H} NMR (471 MHz, DMSO-d₆) δ−113.42 (s, 1F). LCMS (m/z): 411 (M+1). Rₜ: 2.762 min.

### (R)-4-(2-(4-Fluorophenyl)-1H-pyrrolo[2,3-b]pyridin-5-yl)-N-(1-hydroxybutan-2-yl)-thiazole-2-carboxamide (10ap) (Compound 62)

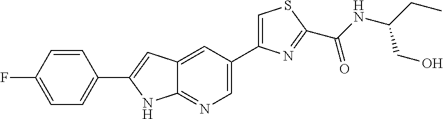 [SMILES: CC[C@H](CO)NC(=O)c1nc(-c2cnc3[nH]c(-c4ccc(F)cc4)cc3c2)cs1]

Synthesized accordingly procedure, described for 6b.

Yield 15% (9 mg). ¹H NMR (500 MHz, DMSO-d₆) δ 12.46-12.20 (m, 1H), 9.01 (d, J=2.1 Hz, 1H), 8.60 (d, J=2.0 Hz, 1H), 8.47-8.39 (m, 2H), 8.06-8.00 (m, 2H), 7.39-7.30 (m, 2H), 6.99 (d, J=2.0 Hz, 1H), 4.82 (t, J=5.6 Hz, 1H), 3.89 (tdd, J=11.1, 7.2, 4.5 Hz, 1H), 3.52 (dh, J=22.0, 5.6 Hz, 2H), 1.69 (dtd, J=14.9, 7.4, 5.1 Hz, 1H), 1.63-1.50 (m, 1H), 0.91 (t, J=7.4 Hz, 3H). ¹⁹F{¹H} NMR (471 MHz, DMSO-d₆) δ−113.42 (s, 1F). LCMS (m/z): 411 (M+1). Rₜ: 2.763 min.

### N-(1,3-Dihydroxypropan-2-yl)-4-(2-(4-fluorophenyl)-1H-pyrrolo[2,3-b]pyridin-5-yl)-thiazole-2-carboxamide (10aq) (Compound 63)

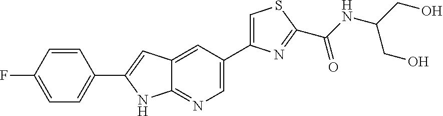 [SMILES: O=C(NC(CO)CO)c1nc(-c2cnc3[nH]c(-c4ccc(F)cc4)cc3c2)cs1]

Synthesized accordingly procedure, described for 6b.

Yield 39% (24 mg). ¹H NMR (500 MHz, DMSO-d₆) δ 12.35 (s, 1H), 8.99 (d, J=2.0 Hz, 1H), 8.59 (d, J=2.1 Hz, 1H), 8.44 (s, 1H), 8.32 (d, J=8.7 Hz, 1H), 8.06-7.98 (m, 2H), 7.38-7.30 (m, 2H), 6.99 (d, J=1.7 Hz, 1H), 4.84 (t, J=5.6 Hz, 2H), 4.00 (dp, J=8.8, 5.8 Hz, 1H), 3.65-3.54 (m, 4H). ¹⁹F{¹H} NMR (471 MHz, DMSO-d₆) δ−113.40 (s, 1F). LCMS (m/z): 413 (M+1). Rₜ: 2.394 min.

### (R)—N-(2,3-dihydroxypropyl)-4-(2-(4-fluorophenyl)-1H-pyrrolo[2,3-b]pyridin-5-yl)-thiazole-2-carboxamide (10ar) (Compound 64)

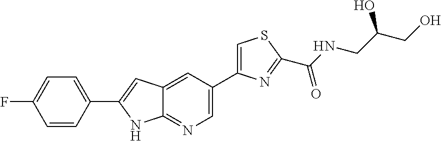 [SMILES: O=C(NC[C@@H](O)CO)c1nc(-c2cnc3[nH]c(-c4ccc(F)cc4)cc3c2)cs1]

Synthesized accordingly procedure, described for 6b.

Yield 39% (24 mg). ¹⁹F{¹H} NMR (471 MHz, DMSO-d₆) δ−113.41. LCMS (m/z): 413 (M+1). Rₜ: 2.409 min.

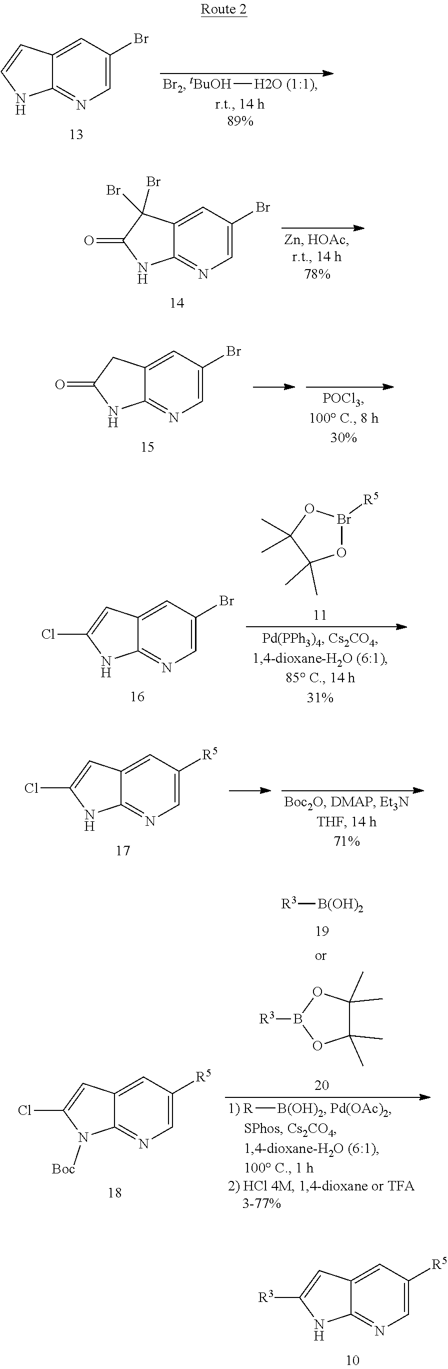

### 3,3,5-Tribromo-1,3-dihydro-2H-pyrrolo[2,3-b]pyridin-2-one (14a)

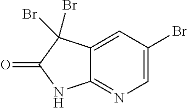 [SMILES: O=C1Nc2ncc(Br)cc2C1(Br)Br]

To a suspension of 5-bromo-1H-pyrrolo[2,3-b]pyridine (13a) (1 eqv., 15.23 mmol, 3.00 g) in tert-butanol (40 ml)—water (40 ml) mixture at room temperature bromine (6.4 eqv., 15.57g, d=3.12 g/ml, 5 ml) was added dropwise. The mixture was stirred in dark overnight at r.t. After the completion of the reaction the reaction mixture was diluted with water, extracted with ether. Organic layer was concentrated giving the desired product.

Yield 89% (5 g). LCMS (m/z): 371 (M+1).

### 5-Bromo-1,3-dihydro-2H-pyrrolo[2,3-b]pyridin-2-one (15a)

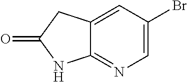 [SMILES: O=C1Cc2cc(Br)cnc2N1]

Zinc powder (9.93 eqv., 53.53 mmol, 3.5g) was added portionwise to the solution of the 3,3,5-tribromo-1,3-dihydro-2H-pyrrolo[2,3-b]pyridin-2-one (14a) (1 eqv., 5.39 mmol, 2.00 g) in glacial acetic acid (40 ml). The reaction mixture was stirred overnight. After the completion the reaction, the solid was filtered off, washed with toluene (50 ml) and triturated and decanted off five times with CH₂Cl₂:MeOH=4:1 (500 ml). The combined solution was treated with 1.0 M aqueous Na₂CO₃ solution (170 ml). Organic layer was separated dried with MgSO₄ and concentrated giving the desired product.

Yield 78%, (894 mg). LCMS (m/z): 213 (M+1).

### 5-Bromo-2-chloro-1H-pyrrolo[2,3-b]pyridine (16a)

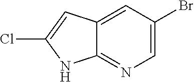 [SMILES: Clc1cc2cc(Br)cnc2[nH]1]

5-bromo-1,3-dihydro-2H-pyrrolo[2,3-b]pyridin-2-one (15a) (3.76 mmol, 800 mg) was heated at 100° C. in phosphoryl chloride (4 ml) for 8 hours. After the completion of the reaction, the reaction mixture was poured on ice (50 ml). When the ice melted, the product was extracted with ethyl acetate. Organic layer was washed with brine, dried with Na₂SO₄ and concentrated under reduced pressure.

Yield 30% (263 mg). LCMS (m/z): 231 (M+1). Rₜ: 2.309 min.

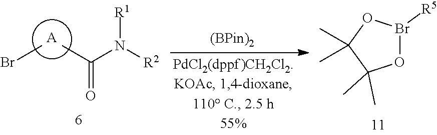

### 4-(4,4,5,5-Tetramethyl-1,3,2-dioxaborolan-2-yl)-N-(2,2,2-trifluoroethyl)thiophene-2-carboxamide (11a)

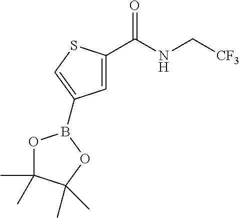 [SMILES: CC1(C)OB(c2csc(C(=O)NCC(F)(F)F)c2)OC1(C)C]

Synthesized accordingly procedure, described for 5a.

Yield 55% (765 mg). LCMS (m/z): 336 (M+1), Rₜ: 2.944 min.

### 4-(2-Chloro-1H-pyrrolo[2,3-b]pyridin-5-yl)-N-(2,2,2-trifluoroethyl)thiophene-2-carboxamide (17a)

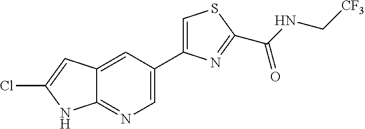 [SMILES: O=C(NCC(F)(F)F)c1nc(-c2cnc3[nH]c(Cl)cc3c2)cs1]

Synthesized accordingly procedure, described for 7a.

Yield 31%, 476 mg. LCMS (m/z): 360 (M+1), Rₜ: 2.702 min.

### tert-Butyl 2-chloro-5-(5-((2,2,2-trifluoroethyl)carbamoyl)thiophen-3-yl)-1H-pyrrolo-[2,3-b]pyridine-1-carboxylate (18a)

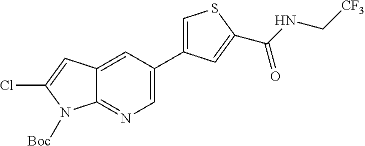 [SMILES: CC(C)(C)OC(=O)n1c(Cl)cc2cc(-c3csc(C(=O)NCC(F)(F)F)c3)cnc21]

To a flame dried flask 4-(2-chloro-1H-pyrrolo[2,3-b]pyridin-5-yl)-N-(2,2,2-trifluoroethyl)-thiophene-2-carboxamide (17a) (1 eqv., 1.32 mmol, 476 mg), di-tert-butyl dicarbonate (1.3 eqv, 1.72 mmol, 375 mg), DMAP (0.1 eqv, 132 umol, 16 mg), Et₃N (1.3 eqv., 1.72 mmol, 174 mg, d=0.726 g/ml, 0.240 ml) and 15 ml of anhydrous THF were added. The flask was backfilled with Ar and reaction mixture was stirred overnight. After the completion of the reaction, the solvent was removed under reduced pressure, crude product was put on silica and purified via flash chromatography (silica, hexane-ethyl acetate gradient, 12% EtOAc 1 CV, 10 CV gradient from 12% EtOAc to 100% EtOAc). The obtained solid was washed with ether and filtered out.

Yield 71% (432 mg). LCMS (m/z): 404 (M+1). Rₜ: 3.393 min.

### 4-(2-(3-Fluorophenyl)-1H-pyrrolo[2,3-b]pyridin-5-yl)-N-(2,2,2-trifluoroethyl)-thiophene-2-carboxamide (Compound 27)

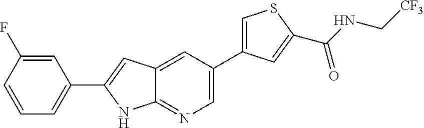 [SMILES: O=C(NCC(F)(F)F)c1cc(-c2cnc3[nH]c(-c4cccc(F)c4)cc3c2)cs1]

Synthesized accordingly procedure, described for 7a.

Yield 24% (14 mg). ¹H NMR (500 MHz, DMSO-d₆) δ 12.34 (s, 1H), 9.16 (t, J=6.3 Hz, 1H), 8.65 (d, J=2.1 Hz, 1H), 8.45 (d, J=1.5 Hz, 1H), 8.25 (d, J=2.2 Hz, 1H), 8.21 (d, J=1.4 Hz, 1H), 7.86-7.82 (m, 2H), 7.53 (td, J=8.2, 6.2 Hz, 1H), 7.20 (td, J=8.6, 2.5 Hz, 1H), 7.10 (s, 1H), 4.13 (dt, J=9.9, 5.0 Hz, 2H). LCMS (m/z): 420 (M+1), Rₜ: 3.072 min.

### N-(2,2,2-Trifluoroethyl)-4-(2-(4-(trifluoromethyl)phenyl)-1H-pyrrolo[2,3-b]pyridin-5-yl)thiophene-2-carboxamide (10at) (Compound 31)

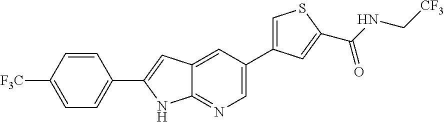 [SMILES: O=C(NCC(F)(F)F)c1cc(-c2cnc3[nH]c(-c4ccc(C(F)(F)F)cc4)cc3c2)cs1]

Synthesized accordingly procedure, described for 7a.

Yield 15% (10 mg). ¹H NMR (500 MHz, DMSO-d₆) δ 12.47 (s, 1H), 9.15 (t, J=6.3 Hz, 1H), 8.67 (d, J=2.1 Hz, 1H), 8.45 (d, J=1.5 Hz, 1H), 8.28 (d, J=2.1 Hz, 1H), 8.22 (d, J=1.4 Hz, 1H), 8.19 (d, J=8.2 Hz, 2H), 7.84 (d, J=8.2 Hz, 2H), 7.18 (s, 1H), 4.14 (qd, J=9.7, 6.2 Hz, 2H). LCMS (m/z): 470 (M+1), Rₜ: 3.291 min

### 4-(2-(4-Methoxyphenyl)-1H-pyrrolo[2,3-b]pyridin-5-yl)-N-(2,2,2-trifluoroethyl)-thiophene-2-carboxamide (10au) (Compound 33)

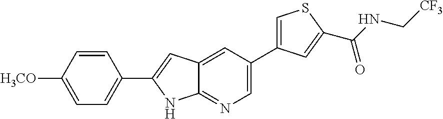 [SMILES: COc1ccc(-c2cc3cc(-c4csc(C(=O)NCC(F)(F)F)c4)cnc3[nH]2)cc1]

Synthesized accordingly procedure, described for 7a.

Yield 13% (8 mg). ¹H NMR (500 MHz, DMSO-d₆) δ 12.15 (s, 1H), 9.14 (t, J=6.3 Hz, 1H), 8.56 (d, J=2.1 Hz, 1H), 8.43 (d, J=1.5 Hz, 1H), 8.17 (dd, J=3.0, 1.7 Hz, 2H), 7.93-7.88 (m, 2H), 7.07-7.02 (m, 2H), 6.85 (d, J=1.5 Hz, 1H), 4.13 (qd, J=9.8, 6.2 Hz, 2H), 3.82 (s, 3H). LCMS (m/z): 432 (M+1). Rₜ: 2.987 min.

### 4-(2-(4-Chlorophenyl)-1H-pyrrolo[2,3-b]pyridin-5-yl)-N-(2,2,2-trifluoroethyl)-thiophene-2-carboxamide (10av) (Compound 32)

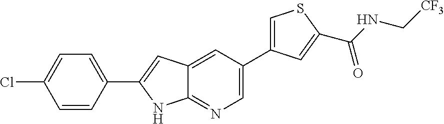 [SMILES: O=C(NCC(F)(F)F)c1cc(-c2cnc3[nH]c(-c4ccc(Cl)cc4)cc3c2)cs1]

Synthesized accordingly procedure, described for 7a.

Yield 10% (6 mg). ¹H NMR (500 MHz, DMSO-d₆) δ 12.33 (s, 1H), 9.15 (t, J=6.3 Hz, 1H), 8.64 (d, J=2.1 Hz, 1H), 8.45 (d, J=1.5 Hz, 1H), 8.23 (dd, J=16.6, 1.8 Hz, 2H), 8.03-7.95 (m, 2H), 7.59-7.52 (m, 2H), 7.04 (s, 1H), 4.14 (qd, J=9.7, 6.1 Hz, 2H). ¹⁹F{¹H}NMR (471 MHz, DMSO-d₆) δ−70.45 (s, 3F). LCMS (m/z): 436 (M+1), Rₜ: 3.232 min

### 4-(2-(2-Fluorophenyl)-1H-pyrrolo[2,3-b]pyridin-5-yl)-N-(2,2,2-trifluoroethyl)-thiophene-2-carboxamide (10aw) (Compound 35)

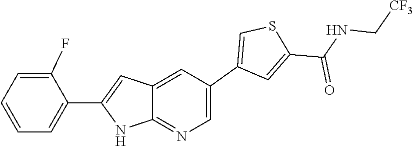 [SMILES: O=C(NCC(F)(F)F)c1cc(-c2cnc3[nH]c(-c4ccccc4F)cc3c2)cs1]

Synthesized accordingly procedure, described for 7a.

Yield 15% (9 mg). ¹H NMR (500 MHz, DMSO-d₆) δ 12.25 (s, 1H), 9.15 (t, J=6.3 Hz, 1H), 8.65 (d, J=2.2 Hz, 1H), 8.43 (d, J=1.5 Hz, 1H), 8.28 (d, J=2.2 Hz, 1H), 8.19 (d, J=1.5 Hz, 1H), 8.01 (td, J=7.9, 1.8 Hz, 1H), 7.47-7.32 (m, 3H), 6.96 (d, J=3.1 Hz, 1H), 4.13 (qd, J=9.7, 6.1 Hz, 2H). LCMS (m/z): 420 (M+1), Rₜ: 3.056 min.

### 4-(2-(4-(2-Morpholinoethoxy)phenyl)-1H-pyrrolo[2,3-b]pyridin-5-yl)-N-(2,2,2-trifluoroethyl)thiophene-2-carboxamide (10ax) (Compound 36)

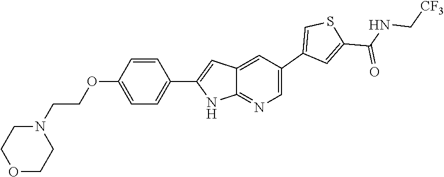 [SMILES: O=C(NCC(F)(F)F)c1cc(-c2cnc3[nH]c(-c4ccc(OCCN5CCOCC5)cc4)cc3c2)cs1]

The 4-(2-chloro-1H-pyrrolo[2,3-b]pyridin-5-yl)-N-(2,2,2-trifluoroethyl)thiophene-2-carboxamide (17a) (1 eqv., 139 umol, 50 mg), 4-(2-(4-(4,4,5,5-tetramethyl-1,3,2-dioxaborolan-2-yl)phenoxy)ethyl)morpholine (11b) (3 eqv., 416 umol, 138 mg), Cs₂CO₃ (1.6 eqv., 222 μm, 72 mg) were put in the flask, solvent mixture 1,4-dioxane (2.4 ml)-water (0.12 ml) mixture was added (20:1). The flask was bubbled with Ar for 5 min, and Pd(OAc)₂ (0.05 eqv., 7 μm, 1 mg) was added. The flask was sealed and the reaction mixture was stirred at 110° C. for 2 h. After the reaction was finished, the solvent was removed under reduced pressure, sample dissolved in DMF and purified on preparative chromatographer.

Yield 9% (7 mg). ¹H NMR (500 MHz, DMSO-d₆) δ 12.14 (s, 1H), 9.14 (t, J=6.3 Hz, 1H), 8.56 (d, J=2.1 Hz, 1H), 8.43 (d, J=1.6 Hz, 1H), 8.18 (d, J=1.5 Hz, 1H), 8.17 (d, J=2.2 Hz, 1H), 7.92-7.86 (m, 2H), 7.09-7.02 (m, 2H), 6.85 (d, J=2.0 Hz, 1H), 4.18-4.08 (m, 4H), 3.62-3.56 (m, 4H), 2.72 (t, J=5.7 Hz, 2H). LCMS (m/z): 531 (M+1), Rₜ: 2.214 min.

### 4-(2-Phenyl-1H-pyrrolo[2,3-b]pyridin-5-yl)-N-(2,2,2-trifluoroethyl)thiophene-2-carboxamide (10ay). (Compound 37)

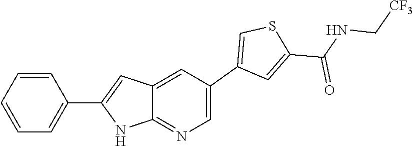 [SMILES: O=C(NCC(F)(F)F)c1cc(-c2cnc3[nH]c(-c4ccccc4)cc3c2)cs1]

Synthesized accordingly procedure, described for 7a.

Yield 11% (6 mg). ¹H NMR (500 MHz, DMSO-d₆) δ 12.28 (s, 1H), 9.16 (t, J=6.3 Hz, 1H), 8.62 (d, J=2.1 Hz, 1H), 8.45 (d, J=1.5 Hz, 1H), 8.23 (d, J=2.2 Hz, 1H), 8.20 (d, J=1.4 Hz, 1H), 8.00-7.94 (m, 2H), 7.49 (t, J=7.7 Hz, 2H), 7.40-7.35 (m, 1H), 7.00 (d, J=1.6 Hz, 1H), 4.14 (qd, J=9.7, 6.1 Hz, 2H). LCMS (m/z): 402 (M+1), Rₜ: 3.007 min.

### 4-(2-(4-(2-(Piperidin-1-yl)ethoxy)phenyl)-1H-pyrrolo[2,3-b]pyridin-5-yl)-N-(2,2,2-trifluoroethyl)thiophene-2-carboxamide (10az) (Compound 38)

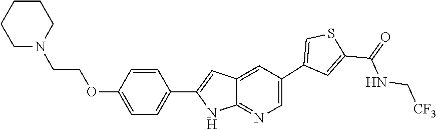 [SMILES: O=C(NCC(F)(F)F)c1cc(-c2cnc3[nH]c(-c4ccc(OCCN5CCCCC5)cc4)cc3c2)cs1]

Synthesized accordingly procedure, described for 10ax.

Yield 9% (4 mg). LCMS (m/z): 529 (M+1). Rₜ: 2.293 min.

### 4-(2-(4-(2-(Piperazin-1-yl)ethoxy)phenyl)-1H-pyrrolo[2,3-b]pyridin-5-yl)-N-(2,2,2-trifluoroethyl)thiophene-2-carboxamide (10ba) (Compound 39)

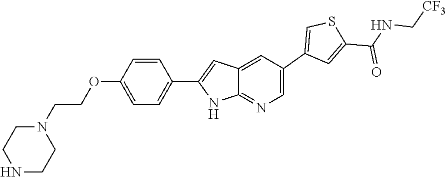 [SMILES: O=C(NCC(F)(F)F)c1cc(-c2cnc3[nH]c(-c4ccc(OCCN5CCNCC5)cc4)cc3c2)cs1]

The tert-butyl 2-chloro-5-(5-((2,2,2-trifluoroethyl)carbamoyl)thiophen-3-yl)-1H-pyrrolo[2,3-b]pyridine-1-carboxylate (18a) (1 eqv., 108 umol, 50 mg), tert-butyl 4-(2-(4-(4,4,5,5-tetramethyl-1,3,2-dioxaborolan-2-yl)phenoxy)ethyl)piperazine-1-carboxylate (11c) (3 eqv., 326 umol, 141 mg), Na₂CO₃ (6 eqv., 652 umol, 69 mg) were put in the flask, solvent mixture 1,4-dioxane (0.8 ml)- water (0.4 ml) mixture was added (2:1). The flask was bubbled with Ar for 5 min, and [1,1′-bis(diphenylphosphino)ferrocene]dichloro-palladium(II), complex with dichloromethane (0.2 eqv., 22 μm, 18 mg) was added. The flask was sealed and the reaction mixture was stirred at 100° C. for 2 h. After the reaction was finished, the solvent was removed under reduced pressure, reaction mixture put on Isolute HM-N celite, and purified via flash chromatography (silica, hexane-ethyl acetate gradient, 12% EtOAc 1 CV, 10 CV gradient from 12% EtOAc to 100% EtOAc). The obtained product was dissolved in 1 ml of TFA and stirred for 1 h. After the reaction was completed, the solvent was removed under reduced pressure sample dissolved in DMF and purified on preparative chromatographer.

Yield 3% (2 mg). LCMS (m/z): 530 (M+1). Rₜ: 2.291 min.

### tert-Butyl 4-(5-(5-((2,2,2-trifluoroethyl)carbamoyl)thiophen-3-yl)-1H-pyrrolo[2,3-b]-pyridin-2-yl)piperidine-1-carboxylate (10bb) (Compound 40)

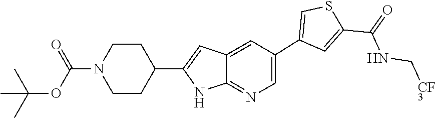 [SMILES: CC(C)(C)OC(=O)N1CCC(c2cc3cc(-c4csc(C(=O)NCC(F)(F)F)c4)cnc3[nH]2)CC1]

The tert-butyl 2-chloro-5-(5-((2,2,2-trifluoroethyl)carbamoyl)thiophen-3-yl)-1H-pyrrolo-[2,3-b]pyridine-1-carboxylate (18a) (1 eqv., 108 umol, 50 mg), tert-butyl 4-(4,4,5,5-tetramethyl-1,3,2-dioxaborolan-2-yl)-3,6-dihydropyridine-1(2H)-carboxylate (11c) (3 eqv., 326 umol, 100 mg), Na₂CO₃ (6 eqv., 652 umol, 69 mg) were put in the flask, solvent mixture 1,4-dioxane (0.8 ml)- water (0.4 ml) mixture was added (2:1). The flask was bubbled with Ar for 5 min, and [1,1′-bis(diphenylphosphino)ferrocene]dichloro-palladium(II), complex with dichloromethane (0.2 eqv., 22 μm, 18 mg) was added. The flask was sealed and the reaction mixture was stirred at 100° C. for 2 h. After the reaction was finished, the solvent was removed under reduced pressure, reaction mixture put on Isolute HM-N celite, and purified via flash chromatography (silica, hexane-ethyl acetate gradient, 12% EtOAc 1 CV, 10 CV gradient from 12% EtOAc to 100% EtOAc). The obtained product was dissolved in methanol (5 ml) and palladium hydroxide on carbon (20 mg) and ammonium formate (140 mg) were added. The flask was sealed and reaction mixture was heated at 80 C for 16 h. After the reaction was completed, the solvent was removed under reduced pressure sample dissolved in DMF and purified on preparative chromatographer.

Yield 7% (4 mg). LCMS (m/z): 509 (M+1). Rₜ: 3.123 min.

### tert-Butyl 5-(5-(5-((2,2,2-trifluoroethyl)carbamoyl)thiophen-3-yl)-1H-pyrrolo[2,3-b]-pyridin-2-yl)-3,6-dihydropyridine-1(2H)-carboxylate (10bc) (Compound 41)

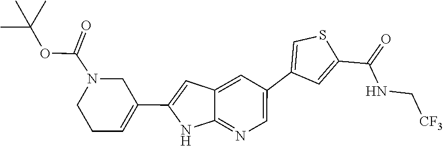 [SMILES: CC(C)(C)OC(=O)N1CCC=C(c2cc3cc(-c4csc(C(=O)NCC(F)(F)F)c4)cnc3[nH]2)C1]

The tert-butyl 2-chloro-5-(5-((2,2,2-trifluoroethyl)carbamoyl)thiophen-3-yl)-1H-pyrrolo-[2,3-b]pyridine-1-carboxylate (18a) (1 eqv., 108 umol, 50 mg), tert-butyl 5-(4,4,5,5-tetramethyl-1,3,2-dioxaborolan-2-yl)-3,6-dihydropyridine-1(2H)-carboxylate (IId) (3 eqv., 326 umol, 100 mg), Na₂CO₃ (6 eqv., 652 umol, 69 mg) were put in the flask, solvent mixture 1,4-dioxane (0.8 ml)- water (0.4 ml) mixture was added (2:1). The flask was bubbled with Ar for 5 min, and [1,1′-bis(diphenylphosphino)ferrocene]dichloro-palladium(II), complex with dichloromethane (0.2 eqv., 22 μm, 18 mg) was added. The flask was sealed and the reaction mixture was stirred at 100° C. for 2 h. After the reaction was finished, the solvent was removed under reduced pressure, reaction mixture put on Isolute HM-N celite, and purified via flash chromatography (silica, Hexane-Ethyl Acetate gradient, 12% EtOAc 1 CV, 10 CV gradient from 12% EtOAc to 100% EtOAc).

Yield 73% (40 mg). LCMS (m/z): 507 (M+1). Rₜ: 3.182 min.

### 4-(2-(3,6-Dihydro-2H-pyran-4-yl)-1H-pyrrolo[2,3-b]pyridin-5-yl)-N-(2,2,2-trifluoro-ethyl)thiophene-2-carboxamide (10bd) (Compound 42)

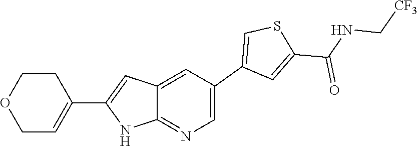 [SMILES: O=C(NCC(F)(F)F)c1cc(-c2cnc3[nH]c(C4=CCOCC4)cc3c2)cs1]

Synthesized accordingly procedure, described for 10ba.

Yield 77% (34 mg). LCMS (m/z): 408 (M+1). Rₜ:2.634 min.

### tert-Butyl 3-(5-(5-((2,2,2-trifluoroethyl)carbamoyl)thiophen-3-yl)-1H-pyrrolo[2,3-b]-pyridin-2-yl)piperidine-1-carboxylate (10be) (Compound 43)

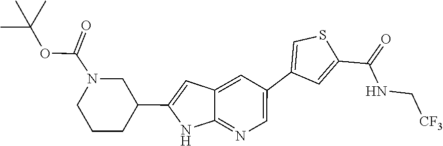 [SMILES: CC(C)(C)OC(=O)N1CCCC(c2cc3cc(-c4csc(C(=O)NCC(F)(F)F)c4)cnc3[nH]2)C1]

The tert-butyl 5-(5-(5-((2,2,2-trifluoroethyl)carbamoyl)thiophen-3-yl)-1H-pyrrolo[2,3-b]-pyridin-2-yl)-3,6-dihydropyridine-1(2H)-carboxylate (10bc) (59 umol, 30 mg), was dissolved in methanol (5 ml) and palladium hydroxide on carbon (20 mg) and ammonium formate (140 mg) were added. The flask was sealed and reaction mixture was heated at 80 C for 16 h. After the reaction was completed, the solvent was removed under reduced pressure sample dissolved in DMF and purified on preparative chromatographer.

Yield 66% (20 mg). LCMS (m/z): 509 (M+1). Rₜ: 3.189 min.

### 4-(2-(1-methylpiperidin-4-yl)-1H-pyrrolo[2,3-b]pyridin-5-yl)-N-(2,2,2-trifluoro-ethyl)thiophene-2-carboxamide (10bf)(Compound 44)

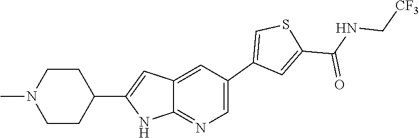 [SMILES: CN1CCC(c2cc3cc(-c4csc(C(=O)NCC(F)(F)F)c4)cnc3[nH]2)CC1]

Synthesized accordingly procedure, described for 10bb.

Yield 28% (7 mg) LCMS (m/z): 423 (M+1). Rₜ.1.929 min.

### 4-(2-(Tetrahydro-2H-pyran-4-yl)-1H-pyrrolo[2,3-b]pyridin-5-yl)-N-(2,2,2-trifluoro-ethyl)thiophene-2-carboxamide (10bg)(Compound 45)

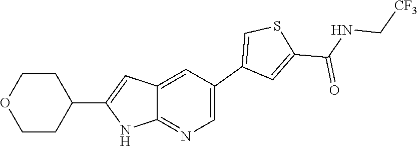 [SMILES: O=C(NCC(F)(F)F)c1cc(-c2cnc3[nH]c(C4CCOCC4)cc3c2)cs1]

Synthesized accordingly procedure, described for 10be.

Yield 37% (4 mg). LCMS (m/z): 410 (M+1). Rₜ.2.544 min.

### 4-(2-(Piperidin-3-yl)-1H-pyrrolo[2,3-b]pyridin-5-yl)-N-(2,2,2-trifluoroethyl)thiophene-2-carboxamide (10bh) (Compound 46)

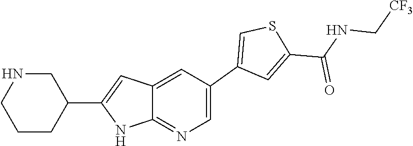 [SMILES: O=C(NCC(F)(F)F)c1cc(-c2cnc3[nH]c(C4CCCNC4)cc3c2)cs1]

The tert-Butyl 3-(5-(5-((2,2,2-trifluoroethyl)carbamoyl)thiophen-3-yl)-1H-pyrrolo[2,3-b]-pyridin-2-yl)piperidine-1-carboxylate (10be) (20 umol, 10 mg) was dissolved in 2 ml 4N HCl solution in 1,4-dioxane and stirred for one hour. After the reaction was completed, the solvent was removed under reduced pressure sample dissolved in DMF and purified on preparative chromatographer.

Yield 50% (4 mg). LCMS (m/z): 409 (M+1). Rₜ: 1.977 min.

### 4-(2-(4-(2-(4-Methylpiperazin-1-yl)ethoxy)phenyl)-1H-pyrrolo[2,3-b]pyridin-5-yl)-N-(2,2,2-trifluoroethyl)thiophene-2-carboxamide (10bi)(Compound 47)

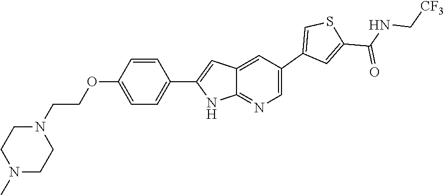 [SMILES: CN1CCN(CCOc2ccc(-c3cc4cc(-c5csc(C(=O)NCC(F)(F)F)c5)cnc4[nH]3)cc2)CC1]

Synthesized accordingly procedure, described for 10ba.

Yield 12% (7 mg). LCMS (m/z): 544 (M+1). Rₜ: 2.224 min.

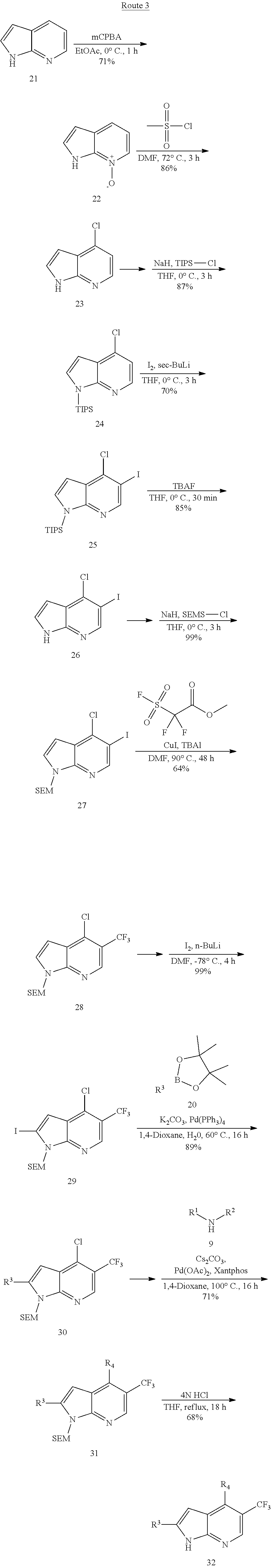

### 1H-Pyrrolo[2,3-b]pyridine 7-oxide (22)

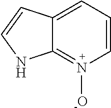

To a stirred solution of 7-azaindole (21) (1 eqv., 42.32 mmol, 5 g) in EtOAc (150 mL) at 0° C. was added mCPBA (1.43 eqv., 60.52 mmol, 70% w/w, 14.9 g) portionwise over a period of 10 min, and the reaction mixture was stirred for another 1 h. After the completion of the reaction, the solid was vacuum filtered and dissolved in chloroform/MeOH (9:1) and neutralized with saturated Na₂CO₃ solution. The layers were separated, and the organic layer was dried over anhydrous Na₂SO₄ and concentrated under reduced pressure to give the 22 as a solid in 71% yield (4.0 g).

The ¹HNMR spectrum correspond to the spectrum that is reported in the literature. (Heinrich, T., et al., Fragment-based discovery of new highly substituted 1H-pyrrolo[2,3-b]- and 3H-imidazolo[4,5-b]-pyridines as focal adhesion kinase inhibitors. J Med Chem, 2013. 56(3): 1160-1170. doi: 10.1021/jm3016014.)

### 4-Chloro-1H-pyrrolo[2,3-b]pyridine (23)

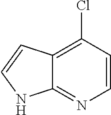

To a stirred solution of 7-azaindole N-oxide (22) (1 eqv., 115.63 mmol, 15.5 g) in DMF (126 mL) at 52° C. was added methane sulfonyl chloride (3 eqv., 346.88 mmol, 26.9 mL) dropwise, and the reaction mixture was heated to 72° C. and stirred at the said temperature for 2 h. After the completion of the reaction, the reaction mixture was poured over crushed ice and neutralized with 5 M NaOH solution. The solid obtained was filtered and dried under vacuum to get the 23 as an orange solid in 84% yield (14.7 g).

The ¹HNMR spectrum correspond to the spectrum that is reported in the literature. (Heinrich, T., et al., J Med Chem, 2013. 56(3): 1160-1170. doi: 10.1021/jm3016014.)

### 4-Chloro-1-(triisopropylsilyl)-1H-pyrrolo[2,3-b]pyridine (24)

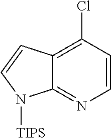

To a stirred solution of 4-chloro-1H-pyrrolo[2,3-b]pyridine (23) (1 eqv., 86.84 mmol, 13.3 g) in THF (248 mL) at 0° C. was added NaH (60% in mineral oil, 1.42 eqv., 123.31 mmol, 4.9 g) portionwise over a period of 15 min. The reaction mixture was then allowed to stir at the same temperature for 2 h when triisopropyl silyl chloride (1.7 eqv., 147.63 mmol, 31.6 mL) was added dropwise (15 min) maintaining the temperature at 0° C. under argon. The mixture was stirred at 0° C. for 3 h when it was allowed to cool to r.t, while stirring for 14 h.

The reaction mixture was quenched with saturated NH₄Cl solution (10 mL) diluted with water and extracted with petroleum ether (3×200 mL). The crude compound was purified by flash chromatography giving 24 as a colorless liquid in 87% yield (23.3 g).

The ¹HNMR spectrum correspond to the spectrum that is reported in the literature. (Heinrich, T., et al., J Med Chem, 2013. 56(3): 1160-1170. doi: 10.1021/jm3016014.)

### 4-Chloro-5-iodo-1-(triisopropylsilyl)-1H-pyrrolo[2,3-b]pyridine (25)

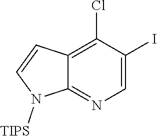

To a solution of 4-chloro-1-(triisopropylsilyl)-1H-pyrrolo[2,3-b]pyridine (24) (1 eqv., 32.37 mmol, 10 g) in THF (90.9 mL) at −78° C. was added sec-BuLi (2.20 eqv., 71.21 mmol, 50.9 mL) dropwise over a period of 30 min, and the resulting suspension was stirred for another 2 h at the given temperature. A solution of iodine (2 eqv., 64.74 mmol, 16.4 g) in THF (45 mL) was then added dropwise over a period of 30 min at the same temperature, and the resulting suspension was stirred for 1 h and slowly brought to 0° C. The reaction was quenched with saturated NH₄Cl solution, extracted with EtOAc, washed with water and brine, dried over Na₂SO₄, and concentrated under reduced pressure to get crude product, which was purified using by flash chromatography to give 25 as a colorless liquid in 70% yield (9.8 g).

The ¹HNMR spectrum correspond to the spectrum that is reported in the literature. (Heinrich, T., et al., Fragment-based discovery of new highly substituted 1H-pyrrolo[2,3-b]- and 3H-imidazolo[4,5-b]-pyridines as focal adhesion kinase inhibitors. J Med Chem, 2013. 56(3): 1160-1170. doi: 10.1021/jm3016014.)

### 4-Chloro-5-iodo-1H-pyrrolo[2,3-b]pyridine (26)

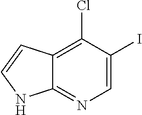 [SMILES: Clc1c(I)cnc2[nH]ccc12]

To a stirred solution of 4-chloro-5-iodo-1-(triisopropylsilyl)-1H-pyrrolo[2,3-b]pyridine (25) (1 eqv., 22.54 mmol, 9.8 g) in anhydrous THF (178 mL) at 0° C. was added TBAF (1 M solution in THF; 24.1 mL, 24.12 mmol) and allowed to stir for 30 min at r.t. After the completion of the reaction, the solvent was evaporated, diluted with EtOAc, washed with water and brine, dried over anhydrous Na₂SO₄, and concentrated under reduced pressure to give the crude compound, which was purified using EtOAc/Hexane by flash chromatography to give 26 as a light blue solid in 85% yield (5.3 g).

The ¹HNMR spectrum correspond to the spectrum that is reported in the literature. (Heinrich, T., et al., J Med Chem, 2013. 56(3): 1160-1170. doi: 10.1021/jm3016014.) 4-Chloro-5-iodo-1-((2-(trimethylsilyl)ethoxy)methyl)-1H-pyrrolo[2,3-b]pyridine (27).

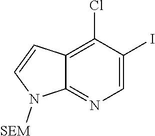 [SMILES: C[Si](C)(C)CCOCn1ccc2c(Cl)c(I)cnc21]

To a solution of 4-chloro-5-iodo-1H-pyrrolo[2,3-b]pyridine (26) (1 eqv., 19.03 mmol, 5.3 g) in THF (57.12 mL) was added NaH (60% dispersion in mineral oil, 1.30 eqv., 24.74 mmol, 0.990 g) portionwise maintaining the temperature at 0° C. The resulting suspension was stirred at 0° C. for another 2 h followed by the addition of SEM-Cl dropwise over a period of 15 min. The suspension was allowed to stir for another 2 h. After the completion of the reaction, it was quenched with saturated ammonium chloride solution, diluted with EtOAc, washed with water and brine, and dried over anhydrous Na₂SO₄. The concentration of the organic layer gave 27 as a pale-yellow oil in 99% yield (7.72g).

The ¹HNMR spectrum correspond to the spectrum that is reported in the literature. (Heinrich, T., et al., J Med Chem, 2013. 56(3): 1160-1170. doi: 10.1021/jm3016014.)

### 4-Chloro-5-(trifluoromethyl)-1-((2-(trimethylsilyl)ethoxy)methyl)-1H-pyrrolo[2,3-b]-pyridine (28)

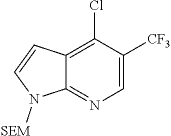 [SMILES: C[Si](C)(C)CCOCn1ccc2c(Cl)c(C(F)(F)F)cnc21]

An flame dried round bottom flask was charged with 4-chloro-5-iodo-1-((2-(trimethylsilyl)ethoxy)methyl)-1H-pyrrolo[2,3-b]pyridine (27) (1 eqv., 15.36 mmol, 6.3 g), Cui (2 eqv., 30.73 mmol, 5.9 g), TBAI (1.50 eqv., 23.05 mmol, 8.5 g), and methyl 2,2-difluoro-2-(fluorosulfonyl)acetate (15 eqv., 230.46 mmol, 44.3 g) and DMF (60.1 mL). The reaction mixture was heated to 90° C. and stir for 16 h at the same temperature. After completion the copper residue was filtered off, and the filtrate was extracted with EtOAc (100 mL), washed with water and brine, and dried over anhydrous Na₂SO₄. The concentration of the organic layer gave the title compound as a colorless solid, which was purified by flash chromatography using EtOAc/Hexane giving 28 in 55% yield (2.9 g). The ¹HNMR spectrum correspond to the spectrum that is reported in the literature. (Heinrich, T., et al., J Med Chem, 2013. 56(3): 1160-1170. doi: 10.1021/jm3016014.)

### 4-Chloro-5-iodo-1-((2-(trimethylsilyl)ethoxy)methyl)-1H-pyrrolo[2,3-b]pyridine (29)

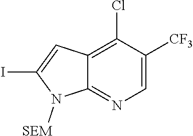 [SMILES: C[Si](C)(C)CCOCn1c(I)cc2c(Cl)c(C(F)(F)F)cnc21]

An oven-dried round bottom flask was charged with 4-chloro-5-(trifluoromethyl)-1-((2-(trimethylsilyl)ethoxy)methyl)-1H-pyrrolo[2,3-b]pyridine (28) (1 eqv., 4.33 mmol, 1.5 g) and THF (16.6 mL). At −78° C., BuLi (2.50 M stock solution in THF, 9.53 mmol was added dropwise to the reaction mixture maintaining the temperature at −78° C. The reaction mixture was stirred for another 2 h at the same temperature, when I₂ (2.5 eqv., 10.83 mmol, 2.75g) in THF (8.3 mL) was added dropwise. The reaction mixture was slowly brought to r.t. After the completion of the reaction, it was quenched with saturated ammonium chloride solution, diluted with EtOAc (60 mL), washed with water and brine, and dried over anhydrous Na₂SO₄. The concentration of the organic layer gave 29 as a dark brown solid in 99% yield (2.05g), which was used without further purification.

The ¹HNMR spectrum correspond to the spectrum that is reported in the literature. (Heinrich, T., et al., J Med Chem, 2013. 56(3): 1160-1170. doi: 10.1021/jm3016014.)

### 4-Chloro-2-(3,6-dihydro-2H-pyran-4-yl)-5-(trifluoromethyl)-1-((2-(trimethylsilyl)-ethoxy)methyl)-1H-pyrrolo[2,3-b]pyridine (30a)

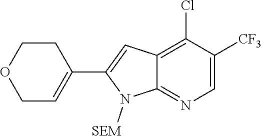 [SMILES: C[Si](C)(C)CCOCn1c(C2=CCOCC2)cc2c(Cl)c(C(F)(F)F)cnc21]

To a microwave vial were added 4-chloro-5-iodo-1-((2-(trimethylsilyl)ethoxy)methyl)-1H-pyrrolo[2,3-b]pyridine (29) (1 eqv., 839.04 mmol, 0.400 g), 2-(3,6-dihydro-2H-pyran-4-yl)-4,4,5,5-tetramethyl-1,3,2-dioxaborolane (1.53 eqv., 1.28 mmol, 0.270 g), tetrakis(triphenyl-phosphine)palladium(0) (0.10 eqv., 0.845 mmol, 0.098 g), potassium carbonate (3.05 eqv., 2.56 mmol, 0.354 g) in 1,4-dioxane:H₂O (4:1, 1.5 mL) under argon. The resulting mixture was stirred at 80° C. for 16 h. The crude product was purified by flash chromatography EtOAc/Hexane to give the 30a as a light brown oil in 89% yield (0.323 g).

LCMS (m/z): 433 (M+1), Rₜ: 4.277 min.

### 2-(3,6-Dihydro-2H-pyran-4-yl)-N-(3-fluorocyclobutyl)-5-(trifluoromethyl)-1-((2-(trimethylsilyl)ethoxy)methyl)-1H-pyrrolo[2,3-b]pyridin-4-amine (31a)

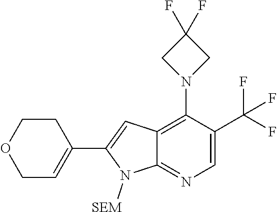 [SMILES: C[Si](C)(C)CCOCn1c(C2=CCOCC2)cc2c(N3CC(F)(F)C3)c(C(F)(F)F)cnc21]

To a microwave vial was added 4-chloro-2-(3,6-dihydro-2H-pyran-4-yl)-5-(trifluoro-methyl)-1-((2-(trimethylsilyl)ethoxy)methyl)-1H-pyrrolo[2,3-b]pyridine (1 eqv., 0.115 mmol, 0.050 g), 3,3-difluoroazetidine (2 eqv., 0.231 mmol, 0.021 g), cesium carbonate (2.47 eqv., 0.285 mmol, 0.093 g) and 1,4-dioxane (0.900 mL). The suspension was degassed and Xantphos (0.22 eqv., 0.025 mmol, 0.015 g) and palladium(II) acetate (0.22 eqv., 0.025 mmol, 0.006 g) were added. The reaction mixture was then degassed again and heated at 100° C. for 12 h. After completion of the reaction, it was filtered through celite, and the filtrate was diluted with EtOAc, washed with brine and H₂O, and dried over anhydrous Na₂SO₄. This was followed by concentration under reduce pressure to give the crude product that was purified by flash chromatography to get 31a as a brown solid in 71% yield (0.040 g).

LCMS (m/z): 490 (M+1), Rₜ: 4.091 min

### 4-(4,4-Difluoropiperidin-1-yl)-2-(3,6-dihydro-2H-pyran-4-yl)-5-(trifluoromethyl)-1-((2-(trimethylsilyl)ethoxy)methyl)-1H-pyrrolo[2,3-b]pyridine (31b)

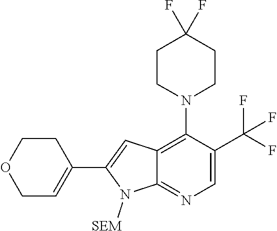 [SMILES: C[Si](C)(C)CCOCn1c(C2=CCOCC2)cc2c(N3CCC(F)(F)CC3)c(C(F)(F)F)cnc21]

Synthesized according to the procedure described for compound 31a.

Yield 17% (0.012g). LCMS (m/z): 518 (M+1), Rₜ: 4.266 min

### 4-(3,3-Difluoroazetidin-1-yl)-2-(3,6-dihydro-2H-pyran-4-yl)-5-(trifluoromethyl)-1H-pyrrolo[2,3-b]pyridine (32a) (Compound86)

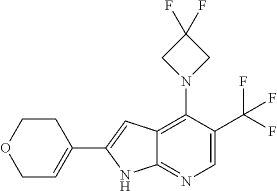 [SMILES: FC1(F)CN(c2c(C(F)(F)F)cnc3[nH]c(C4=CCOCC4)cc23)C1]

To a solution of 4-(3,3-difluoroazetidin-1-yl)-2-(3,6-dihydro-2H-pyran-4-yl)-5-(trifluoro-methyl)-1H-pyrrolo[2,3-b]pyridine (31a) (40 mg, 0.082 mmol) in THF (1.68 mL) was added HCl solution in 1,4-dioxane (4 N, 0.700 mL) and heated at reflux for 5 h. After completion of the reaction, the solvent was evaporated; the residue was dissolved in EtOAc (15 mL) and neutralized with saturated Na₂CO₃ solution. The organic layer was separated, washed with water and brine, and dried over anhydrous Na₂SO₄ and evaporated. The residue was purified by column chromatography to get 32a as a brown solid in 68% yield (0.020 g).

¹H NMR (500 MHz, DMSO-d₆) δ 11.99 (s, 1H), 8.17 (s, 1H), 6.65 (d, J=2.2 Hz, 1H), 6.45 (s, 1H), 4.91 (t, J=12.6 Hz, 4H), 4.25 (q, J=2.8 Hz, 2H), 3.83 (q, J=6.1, 5.5 Hz, 2H), 2.51-2.44 (m, 2H). ¹⁹F{¹H} NMR (471 MHz, DMSO-d₆) δ−50.59 (s, 2F) −101.20 (s, 3F).

LCMS (m/z): 360 (M+1), Rₜ: 2.586 min.

### 4-(4,4-Difluoropiperidin-1-yl)-2-(3,6-dihydro-2H-pyran-4-yl)-5-(trifluoromethyl)-1H-pyrrolo[2,3-b]pyridine (32b) (Compound 93)

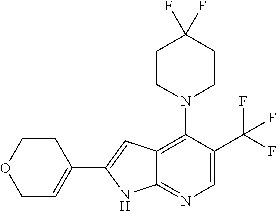 [SMILES: FC1(F)CCN(c2c(C(F)(F)F)cnc3[nH]c(C4=CCOCC4)cc23)CC1]

Synthesized according to the procedure described for compound 32a.

Yield 89%, 8 mg. ¹H NMR (500 MHz, DMSO-d₆) δ 11.99 (s, 1H), 8.17 (s, 1H), 6.65 (d, J=2.2 Hz, 1H), 6.45 (s, 1H), 4.91 (t, J=12.6 Hz, 4H), 4.25 (q, J=2.8 Hz, 2H), 3.83 (q, J=6.1, 5.5 Hz, 2H), 2.51-2.44 (m, 2H). LCMS (m/z): 360 (M+1), Rₜ: 2.586 min.

### Biological Activity of ULK1 Inhibitors

Activity-based ULK1 kinase via radiometric HotSpot™ kinase assay (Reaction Biology) was used to determine enzymatic activity (IC₅₀).

| | Binding Potential | | | |
|---|---|---|---|---|
| | | IC₅₀ (nM) | ULK1 | ULK1 |
| | | Radiometric | LanthaScreen | NanoBRET |
| Cmpd # | ΔTₘ (° C.) | HotSpot | IC₅₀ (nM) | EC₅₀ (nM) |
| 1 | 7.95 | 40 | 9 | 57 |
| 2 | 8.04 | 23 | 28 | 252 |
| 3 | 0.29 | 10000 | | |
| 4 | 1.95 | 818 | 2456 | |
| 5 | 0.58 | 10000 | | |
| 6 | 5.83 | 113 | 214 | |
| 7 | 2.55 | 1613 | 5254 | |
| 8 | 6.11 | 81 | 184 | |
| 9 | 1.29 | 10000 | | |
| 10 | 5.07 | 139 | 375 | |
| 11 | 6.06 | 79 | 210 | |
| 12 | 2.88 | 198 | 540 | |
| 13 | 6 | 17 | 107 | |
| 14 | 1.07 | 3642 | 6856 | |
| 15 | 6.34 | 2 | 4 | |
| 16 | 1.02 | 5563 | 10000 | |
| 17 | 2.75 | 200 | 1287 | |
| 18 | 3.86 | 60 | 2475 | |
| 19 | 10.62 | 7 | 25 | 186 |
| 20 | 0.72 | 10000 | | |
| 21 | 8.48 | 10 | 10 | 61 |
| 22 | 0.73 | 4147 | 4400 | |
| 23 | 3.42 | 437 | 1340 | |
| 24 | 0.4 | 10000 | | |
| 25 | 7.37 | 34 | 88 | |
| 26 | 6.5 | 14 | 21 | 292 |
| 27 | 2.55 | 16 | 27 | 357 |
| 28 | 4.01 | 1087 | 1938 | |
| 29 | 0.9 | 938 | 1225 | |
| 30 | 1.79 | 1897 | 10000 | |
| 31 | 4.81 | 102 | 151 | |
| 32 | 5.67 | 42 | 101 | |
| 33 | 8.7 | 5 | 23 | 95 |
| 34 | 2.79 | 2163 | 10000 | |
| 35 | 6.18 | 15 | 100 | 368 |
| 36 | 12.82 | 3 | 8 | 22 |
| 37 | 5.13 | 5 | 21 | 218 |
| 38 | 12.82 | 15 | 14 | 22 |
| 39 | 16.23 | 4 | 13 | 27 |
| 40 | 1.63 | 10000 | | |
| 41 | 9.89 | 333 | 75 | |
| 42 | 15.07 | 6 | 2 | |
| 43 | 4.2 | 10000 | | |
| 44 | 11.08 | 73 | 78 | |
| 45 | 7.71 | 94 | 124 | |
| 46 | 11.18 | 96 | 114 | |
| 47 | 13.65 | 5 | 8 | 13 |
| 48 | 4.52 | 261 | 475 | |
| 49 | 5.11 | 341 | 543 | |
| 50 | 4.4 | 91 | 86 | |
| 51 | 4.76 | 184 | 387 | |
| 52 | 8.86 | 45 | 106 | |
| 53 | 3.11 | 66 | 94 | |
| 54 | 1.62 | 1470 | 5853 | |
| 55 | 8.56 | 44 | 74 | |
| 56 | 6.85 | 72 | 80 | |
| 57 | 8.73 | 49 | 20 | |
| 58 | 9.22 | 22 | 67 | 2369 |
| 59 | 11.03 | 7 | 10 | 149 |
| 60 | 8.11 | 14 | 35 | 1373 |
| 61 | 9.31 | 8 | 7 | 131 |
| 62 | 4.81 | 78 | 182 | |
| 63 | 6.46 | 55 | 103 | |
| 64 | 7.54 | 41 | 58 | 961 |
| 65 | 7.12 | 29 | 17 | 122 |
| 66 | 14.72 | 2 | 3 | 43 |
| 67 | 15.47 | 2 | 2 | 10 |
| 68 | 13.05 | 2 | 3 | 10 |
| 69 | 14.5 | 3 | 5 | 44 |
| 70 | 8.04 | 22 | 8 | 129 |
| 71 | 3.73 | 14 | 39 | 414 |
| 72 | 7.96 | 50 | 36 | |
| 73 | 7.72 | 10 | 3 | 195 |
| 74 | 1.87 | 644 | 665 | |
| 75 | 3 | 10000 | | |
| 76 | 7.56 | 44 | 25 | 541 |
| 77 | 4.54 | 348 | 741 | |
| 78 | 4.83 | 20 | 27 | 282 |
| 79 | 5.45 | 12 | 16 | 156 |
| 80 | 4.99 | 502 | 966 | |
| 81 | 5.41 | 15 | 14 | |
| 82 | 4.39 | 9 | 24 | |
| 83 | 6.81 | 401 | 208 | |
| 84 | 7.13 | 549 | 412 | |
| 85 | 0.54 | 10000 | | |
| 86 | 6.82 | 56 | 172 | |
| 87 | 12.48 | 10 | 16 | 76 |
| 88 | 2.37 | 4446 | 4517 | |
| 89 | 8.13 | 37 | 92 | 1160 |
| 90 | 9.23 | 155 | 145 | |
| 91 | 9.77 | 110 | 167 | |
| 92 | 0.88 | 10000 | | |
| 93 | 2.03 | 7073 | 8279 | |
| 94 | 0.76 | 10000 | | |
| 95 | 0.49 | 758 | 917 | |
| 96 | 3.09 | 10000 | | |
| 97 | 7.07 | 2381 | 541 | |
| 98 | 10.3 | 144 | 57 | 184 |
| 99 | 7.77 | 625 | 374 | |
| 100 | 4.99 | 171 | 218 | |
| 101 | 10.99 | 131 | 50 | 157 |
| 102 | | | 15 | 952 |
| 103 | | | 36 | 1396 |
| 104 | | | 29 | 4294 |
| 105 | | | 139 | 10000 |
| 106 | | | 16 | 1132 |
| 107 | | | 19 | 391 |
| 108 | | | 26 | 219 |
| 109 | | | 80 | |
| 110 | | | 54 | 1788 |
| 111 | | | 10000 | |
| 112 | | | 8 | |
| 113 | | | 63 | |
| 114 | | | 14 | |
| 115 | | | 81 | |
| 116 | | | 201 | |
| 117 | | | 85 | |
| 118 | | | 126 | |
| 119 | | | 1600 | |
| 120 | | | 1211 | |
| 121 | | | 320 | |
| 140 | | | 1 | 86 |
| 141 | | | 884 | |
| 142 | | | 88 | |

Expression and Purification of ULK1 The protein expression construct of ULK1 kinase domain (1-283) was cloned with an N-terminal 61His-Sumo tag into pET28a(+) vector (Genescript). Another construct ULK1 1-283^{E37A/K38A} which has mutations at Glutamate 37 and Lysine 38 to alanine was also made for protein crystallization. To induce ideal protein expression of ULK1 1-283, a plasmid containing lambda phosphatase in pGEX6P2 was transformed into the BL21 star cells together with ULK1 1-283 constructs. These cells were grown in TB medium with 50 mg/mi kanacmycin, 50 mg/ml carbenicillin to OD600 of 0.8 at 25° C., then induced with 0.5 ml IPTG for growth overnight at 20° C. After harvest, cell pellet was resuspended in buffer (50 mM Hepes pH7.5, 300 mM NaCl, 10 mM MgCl₂, 20% Glycerol, 0.5 mM TCEP) and lysed with homogenizer. The 6H-is-sumo tagged ULK1 1-283 was then purified by a Ni-NTA column. The 6His-sumo tag can be cleaved by sumo protease at 4° C. in 2 hours and the untagged ULK1 1-283 can be further purified by another Ni-NTA column. The final protein was purified by size exclusion chromatography using a Superdex 75 26/60 column (GE Lifescience) and concentrated to 20 mg/mL in buffer 50 mM TBS, pH 7.5, 100 mM NaCl, 10 mM MgCl2, 10% Glycerol.

### Differential Scanning Fluorimetry

20 μL of a premix solution composed of reaction buffer (50 mM Hepes, pH7.5, 150 mM NaCl, 10% Glycerol) with 2 μM of ULK1 kinase domain, 50 μM of tested compounds, 2% DMSO and 5× Sypro Orange dye (Invitrogen) was added to the wells of a MicroAmp® Fast 96 well reaction plate (Applied Biosystems). Each condition was tested in quadruplicate. The wells were mixed by pipetting, sealed with MicroAmp™ Optical Adhesive Film (Applied Biosystem), centrifuged at 1000×g for 30 seconds and heated on a Step One Plus RT-PCR machine (Applied Biosystem). The reaction was set for 25° C. to 95° C. with 1° C./minute increments after an initial incubation at 25° C. for 10 minutes.

Fluorescence was measure with an excitation wavelength of 492 nM and an emission wavelength of 610 nM. The results were analysis by Protein Thermo Shift™ software version 1.3 (Applied Biosystem). The observed thermal shift (ΔTₘ) was recorded as the difference between the Tₘ of the sample and the DMSO reference wells.

NanoBRET. Bioluminescence resonance energy transfer (BRET), specifically nanoBRET (Promega) was used as the method to quantify target engagement (EC₅₀) in live cells.

Briefly, 1.6×10⁵ H2030 cells (ATCC) were transfected using Fugene HD (Promega) with 20 μg/ml of ULK1-nanoluc (Promega) and seeded at 4000 cells per well of a 384 well plate (BeckmanCoulter) and cells were incubated at 37 C/5% CO₂ in a humidified tissue culture incubator for 24 hr. DMSO or compound (10 point, 3-fold dilutions starting from 10% M for each compound) was added to assay plates using an ECH0665 workstation (BeckmanCoulter). Next, NanoBret Tracer K10 (2.5 μl) was added to each well and incubated for a further 2 hr at 37 C/5% CO₂ in a humidified tissue culture incubator. To measure bioluminescence, 12.5 μl of NanoGlo substrate was administered to each well and signal detected using an Envision (PerkinElmer) plate reader. Data was normalized to DMSO treated wells and plotted using GraphPad Prism (four-parameter logistic fit).

LanthaScreen. ULK1 inhibitor (IC₅₀) values were were determined in-house using LanthaScreen™ (PerkinElmer) technology.. Briefly, final assay concentrations of GST-ULK1, LanthaScreen® Tracer substrate (Promega) and ATP were 5 nM, 30 nM and 20 μM respectively. The reaction was performed at room temperature in a 10 μl final volume (384-well plate, Beckman Coulter) containing the following 50 mM Hepes (pH7.5), 5 mM MgCl₂, 1 mM di-thiothreitol, 0.01% triton-100 and 1% DMSO. After 1 hr the reaction was terminated by the addition of 10 μl anti-GST coupled Eu-Labeled LanthaScreen® antibody (donor) in Lance detection buffer (PerkinElmer). The fluorescent signal was detected using an Envision (PerkinElmer) plate reader. 10-point concentration response curves with 3-fold dilutions starting from 10% M for each compound was generated in duplicate and plotted using GraphPad Prism (four-parameter logistic fit).

### REFERENCES

The following references are incorporated by reference herein in their entirety for all purposes:
1. Klionsky, D. J. and S. D. Emr, Autophagy as a regulated pathway of cellular degradation. Science, 2000. 290 (5497): p. 1717-21. doi: 10.1126/science.290.5497.1717. PubMed PMID: 11099404; PMCID: PMC2732363.
2. Yorimitsu, T. and D. J. Klionsky, Autophagy: molecular machinery for self-eating. Cell Death Differ, 2005. 12 Suppl 2: p. 1542-52. doi: 10.1038/sj.cdd.4401765. PubMed PMID: 16247502; PMCID: PMC1828868.
3. De Duve, C. and R. Wattiaux, Functions of lysosomes. Annu Rev Physiol, 1966. 28: p. 435-92. doi: 10.1146/annurev.ph.28.030166.002251. PubMed PMID: 5322983.
4. Tooze, S. A. and T. Yoshimori, The origin of the autophagosomal membrane. Nat Cell Biol, 2010. 12 (9): p. 831-5. doi: 10.1038/ncb0910-831. PubMed PMID: 20811355.
5. Rubinsztein, D. C., P. Codogno, and B. Levine, Autophagy modulation as a potential therapeutic target for diverse diseases. Nat Rev Drug Discov, 2012. 11 (9): p. 709-30. doi: 10.1038/nrd3802. PubMed PMID: 22935804; PMCID: PMC3518431.
6. White, E. J., et al., Autophagy regulation in cancer development and therapy. Am J Cancer Res, 2011. 1 (3): p. 362-372. PubMed PMID: 21969237; PMCID: PMC3180058.
7. Takamura, A., et al., Autophagy-deficient mice develop multiple liver tumors. Genes Dev, 2011. 25 (8): p. 795-800. doi: 10.1101/gad.2016211. PubMed PMID: 21498569; PMCID: PMC3078705.
8. Guo, J. Y., B. Xia, and E. White, Autophagy-mediated tumor promotion. Cell, 2013. 155(6): p. 1216-9. doi: 10.1016/j.cell.2013.11.019. PubMed PMID: 24315093; PMCID: PMC3987898.
9. Strohecker, A. M., et al., Autophagy sustains mitochondrial glutamine metabolism and growth of BrafV600E-driven lung tumors. Cancer Discov, 2013. 3(11): p. 1272-85. doi: 10.1158/2159-8290.CD-13-0397. PubMed PMID: 23965987; PMCID: PMC3823822.
10. Bryant, K. L., et al., Combination of ERK and autophagy inhibition as a treatment approach for pancreatic cancer. Nat Med, 2019. 25(4): p. 628-640. doi: 10.1038/s41591-019-0368-8. PubMed PMID: 30833752; PMCID: PMC6484853.
11. Kinsey, C. G., et al., Protective autophagy elicited by RAF→MEK→ERK inhibition suggests a treatment strategy for RAS-driven cancers. Nat Med, 2019. 25(4): p. 620-627. doi: 10.1038/s41591-019-0367-9. PubMed PMID: 30833748; PMCID: PMC6452642.
12. Lee, C. S., et al., MAP kinase and autophagy pathways cooperate to maintain RAS mutant cancer cell survival. Proc Natl Acad Sci USA, 2019. 116(10): p. 4508-4517. doi: 10.1073/pnas.1817494116. PubMed PMID: 30709910; PMCID: PMC6410784.
13. Chan, E. Y. and S. A. Tooze, Evolution of Atg1 function and regulation. Autophagy, 2009. 5(6): p. 758-65. doi: 10.4161/auto.8709. PubMed PMID: 19411825.
14. Kim, J., et al., AMPK and mTOR regulate autophagy through direct phosphorylation of Ulk1. Nat Cell Biol, 2011. 13(2): p. 132-41. doi: 10.1038/ncb2152. PubMed PMID: 21258367; PMCID: PMC3987946.
15. Ganley, I. G., et al., ULK1.ATG13.FIP200 complex mediates mTOR signaling and is essential for autophagy. J Biol Chem, 2009. 284(18): p. 12297-305. doi: 10.1074/jbc.M900573200. PubMed PMID: 19258318; PMCID: PMC2673298.
16. Hosokawa, N., et al., Nutrient-dependent mTORC1 association with the ULK1-Atg13-FIP200 complex required for autophagy. Mol Biol Cell, 2009. 20(7): p. 1981-91. doi: 10.1091/mbc.E08-12-1248. PubMed PMID: 19211835; PMCID: PMC2663915.
17. Jung, C. H., et al., ULK-Atg13-FIP200 complexes mediate mTOR signaling to the autophagy machinery. Mol Biol Cell, 2009. 20(7): p. 1992-2003. doi: 10.1091/mbc.E08-12-1249. PubMed PMID: 19225151; PMCID: PMC2663920.
18. Egan, D. F., et al., Phosphorylation of ULK1 (hATG1) by AMP-activated protein kinase connects energy sensing to mitophagy. Science, 2011. 331(6016): p. 456-61. doi: 10.1126/science.1196371. PubMed PMID: 21205641; PMCID: PMC3030664.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound of Formula I or Formula II: 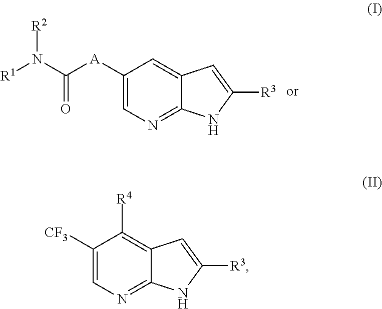
or a pharmaceutically acceptable salt, solvate, prodrug, or derivative thereof, wherein:
A is selected from: 6- to 10-membered monocyclic or bicyclic aryl; and 5- to 10-membered monocyclic or bicyclic heteroaryl; wherein A is optionally substituted with one or more groups selected from X;
R¹ is selected from C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups selected from X;
R² is selected from hydrogen, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups selected from X;
or R¹ and R² are brought together with the nitrogen atom to which they are attached to form a 3- to 8-membered monocyclic or bicyclic heterocycle or a 5- to 10-membered monocyclic or bicyclic heteroaryl, each of which is optionally substituted with one or more groups selected from X;
R³ is selected from: C₃-C₈ monocyclic or bicyclic cycloalkyl; C₃-C₆ cycloalkenyl; 3- to 8-membered monocyclic or bicyclic heterocycle; 6- to 10-membered monocyclic or bicyclic aryl; and 5- to 10-membered monocyclic or bicyclic heteroaryl; wherein R³ is optionally substituted with one or more groups selected from X; or
R³ is —B—C;
B is selected from: C₃-C₈ monocyclic or bicyclic cycloalkyl; C₃-C₆ cycloalkenyl; 3- to 8-membered monocyclic or bicyclic heterocycle; wherein B is optionally substituted with one or more groups selected from X;
C is 3- to 8-membered monocyclic or bicyclic heterocycle which is optionally substituted with one or more groups selected from X;
R⁴ is selected from OR⁵ and NR⁶R⁷;
R⁵ is selected from C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups selected from X;
R⁶ is selected from hydrogen, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups selected from X;
R⁷ is selected from C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups selected from X;
or R⁶ and R⁷ are brought together with the nitrogen atom to which they are attached to form a 3- to 8-membered monocyclic or bicyclic heterocycle or a 5- to 10-membered monocyclic or bicyclic heteroaryl, each of which is optionally substituted with one or more groups selected from X;
X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which is optionally substituted with one or more groups selected from Y;
R^{x} and R^{Y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

2. A compound of claim 1, wherein R³ is selected from C₃-C₈ monocyclic or bicyclic cycloalkyl, C₃-C₆ cycloalkenyl, 3- to 8-membered monocyclic or bicyclic heterocycle, and 6- to 10-membered monocyclic or bicyclic aryl, each of which is optionally substituted with 1, 2, 3, or 4 groups selected from X.

3-5. (canceled)

6. A compound of claim 1, wherein R³ is selected from:

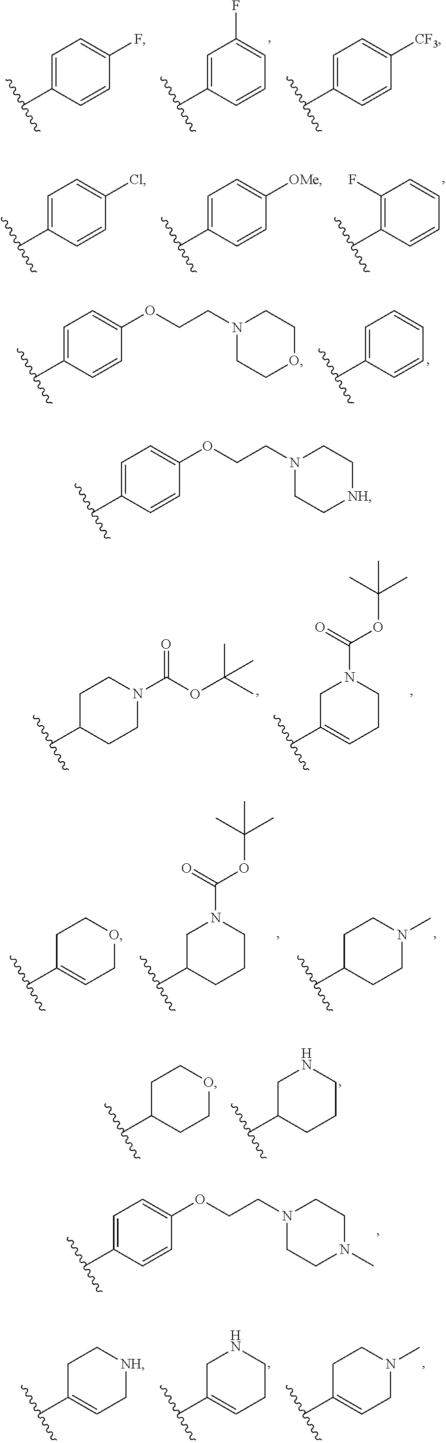

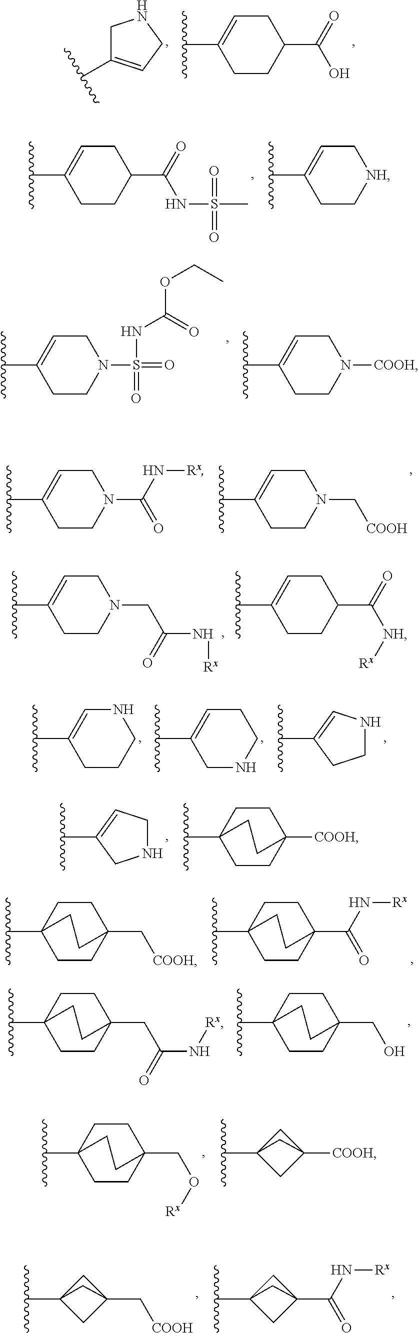

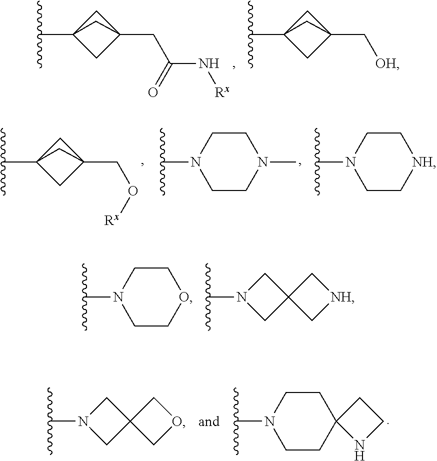

7. (canceled)

8. A compound of claim 1, wherein R³ is —B—C.

9. A compound of claim 8, wherein B is selected from C₃-C₈ monocyclic or bicyclic cycloalkyl, C₃-C₆ cycloalkenyl, and 3- to 8-membered monocyclic or bicyclic heterocycle, each of which is optionally substituted with 1, 2, 3, or 4 groups selected from X.

10-11. (canceled)

12. A compound of claim 8, wherein C is 3- to 8-membered monocyclic or bicyclic heterocycle optionally substituted with 1, 2, 3, or 4 groups selected from X.

13. A compound of claim 1, wherein R³ is selected from:

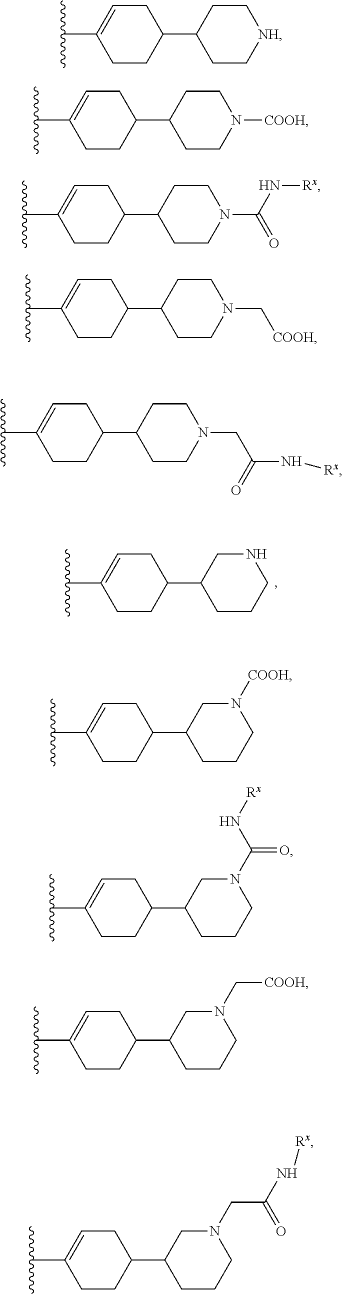

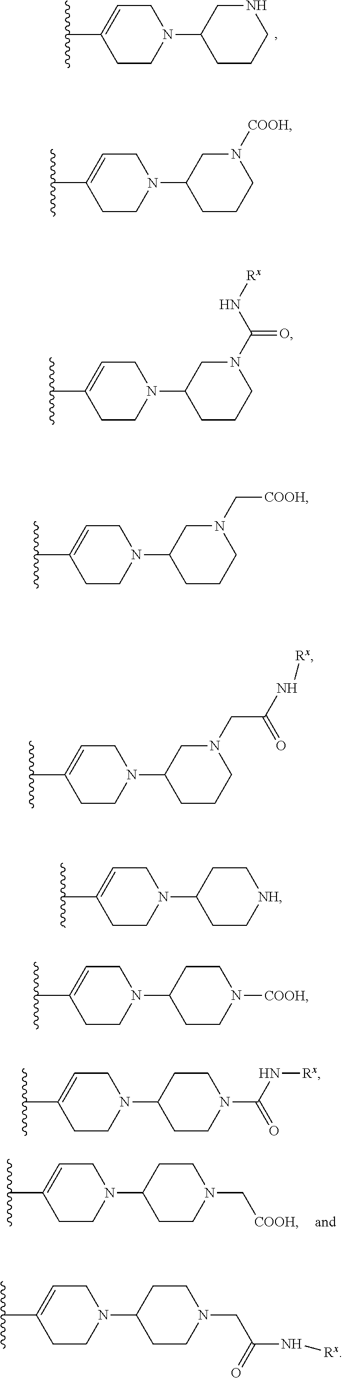

14. A compound of claim 1, wherein A is selected from 6- to 10-membered monocyclic or bicyclic aryl and 5- to 10-membered monocyclic or bicyclic heteroaryl, each of which is optionally substituted with 1, 2, 3, or 4 groups selected from X.

15. (canceled)

16. A compound of claim 1, wherein A is selected from:

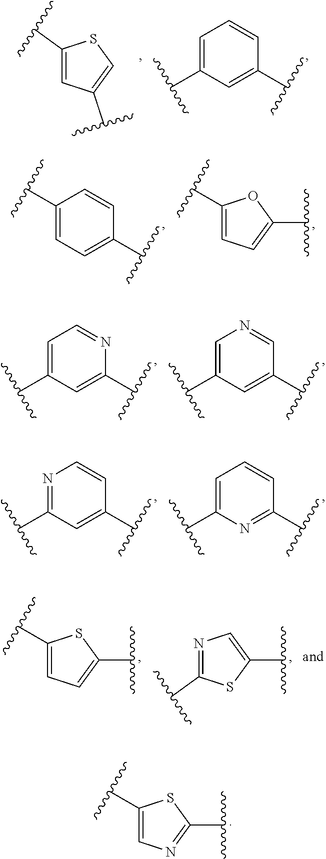

17. A compound of claim 1, wherein R² is hydrogen, and
R¹ is selected from:

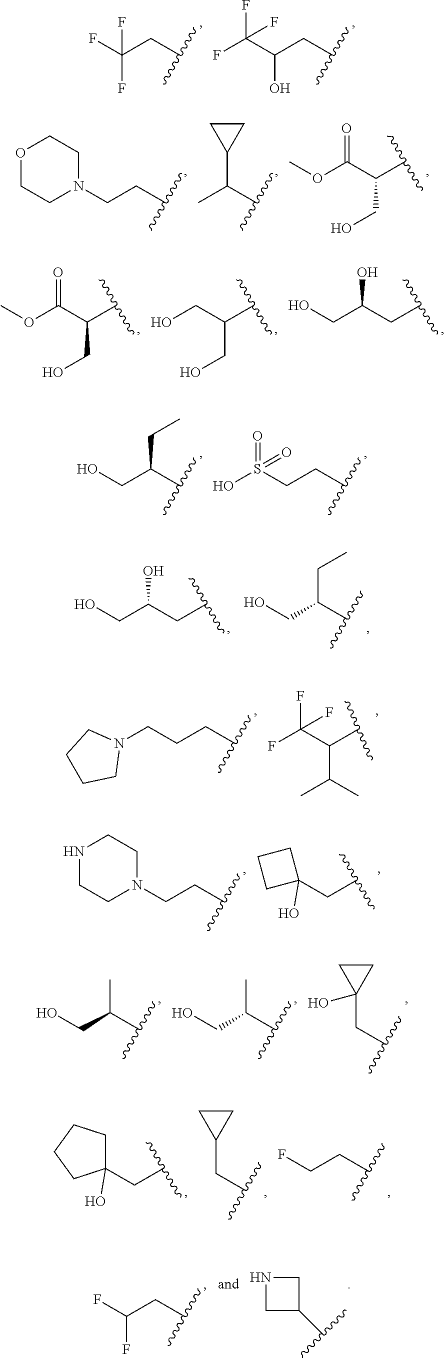

18. (canceled)

19. A compound of claim 1, wherein R¹ and R² are brought together with the nitrogen to which they are attached to form a group selected from:

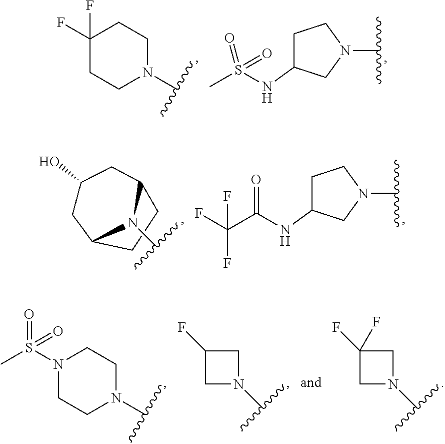

20. A compound of claim 1, wherein R⁴ is NR⁶R⁷.

21. A compound of claim 20, wherein R⁶ is hydrogen, and R⁷ is selected from C₁-C₆ alkyl, C₁-C₆ haloalkyl, (C₃-C₆ cycloalkyl)(C₀-C₃ alkyl)-, and (3- to 8-membered monocyclic or bicyclic heterocycle)-(C₀-C₃ alkyl)-, each of which is optionally substituted with 1, 2, 3, or 4 groups selected from X.

22-25. (canceled)

26. A compound of claim 20, wherein R⁶ and R⁷ are brought together with the nitrogen atom to which they are attached to form a 3- to 8-membered monocyclic or bicyclic heterocycle optionally substituted with 1, 2, 3, or 4 groups selected from X.

27. A compound of claim 1, wherein R⁴ is selected from:

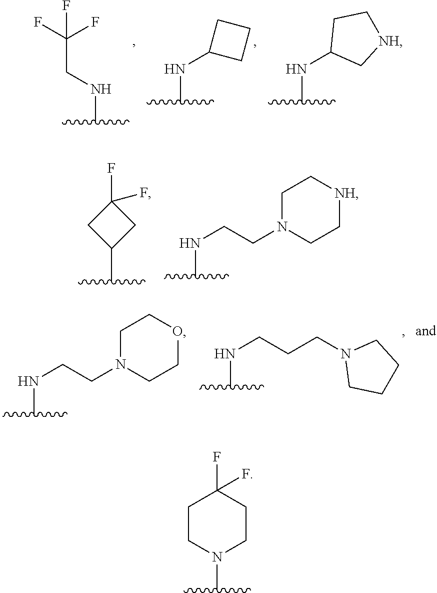

28. A compound of Formula Ia or Formula IIa 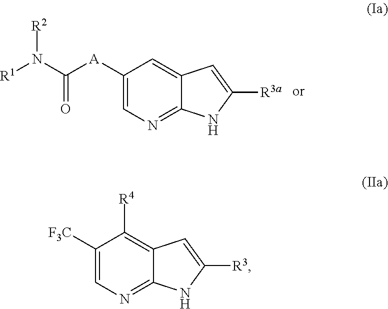
or a pharmaceutically acceptable salt, solvate, prodrug, or derivative thereof, wherein:
A is selected from: 6- to 10-membered monocyclic or bicyclic aryl; and 5- to 10-membered monocyclic or bicyclic heteroaryl; wherein A is optionally substituted with one or more groups selected from X;
R¹ is selected from C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups selected from X;
R² is selected from hydrogen, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, each of which is optionally substituted with one or more groups selected from X;
or R¹ and R² are brought together with the nitrogen atom to which they are attached to form a 3- to 8-membered monocyclic or bicyclic heterocycle or a 5- to 10-membered monocyclic or bicyclic heteroaryl, each of which is optionally substituted with one or more groups selected from X;
R³ is selected from: C₃-C₈ monocyclic or bicyclic cycloalkyl; C₃-C₆ cycloalkenyl; 3- to 8-membered monocyclic or bicyclic heterocycle; 6- to 10-membered monocyclic or bicyclic aryl;
and 5- to 10-membered monocyclic or bicyclic heteroaryl; wherein R³ is optionally substituted with one or more groups selected from X; or
R³ is —B—C;
B is selected from: C₃-C₈ monocyclic or bicyclic cycloalkyl; C₃-C₆ cycloalkenyl; 3- to 8-membered monocyclic or bicyclic heterocycle; wherein B is optionally substituted with one or more groups selected from X;
C is 3- to 8-membered monocyclic or bicyclic heterocycle which is optionally substituted with one or more groups selected from X;
R^{3a} is —B—Ca;
C^{a} is C₃-C₆ cycloalkyl which is optionally substituted with one or more groups selected from X;
R^{4a} is C₃-C₆ cycloalkyl, 6- to 10-membered monocyclic or bicyclic aryl, or 5- to 10-membered monocyclic or bicyclic heteroaryl, which is optionally substituted with one or more groups selected from X;
X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which is optionally substituted with one or more groups selected from Y;
R^{x} and R^{Y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

29. A compound selected 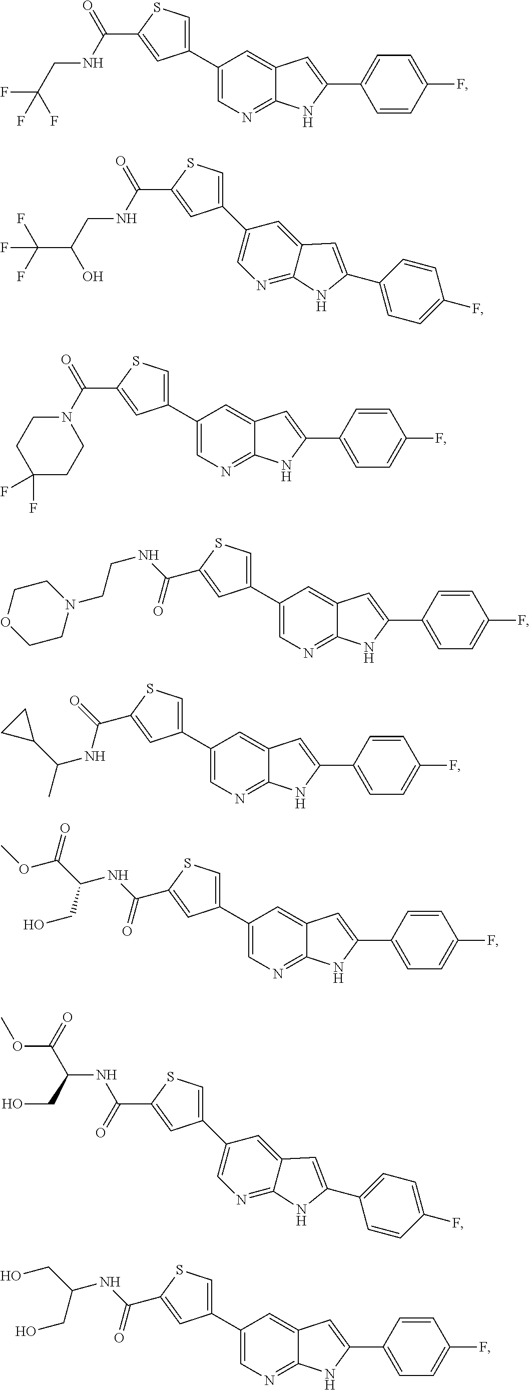 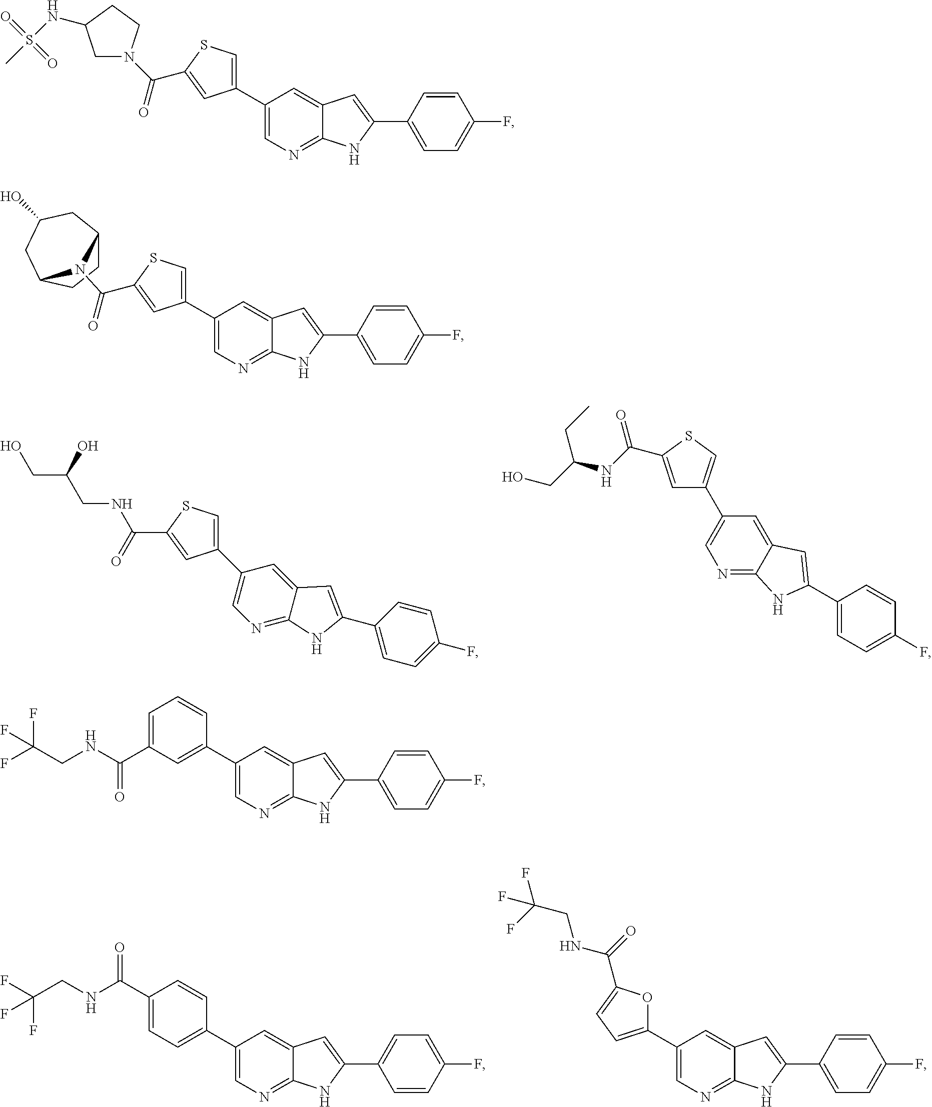 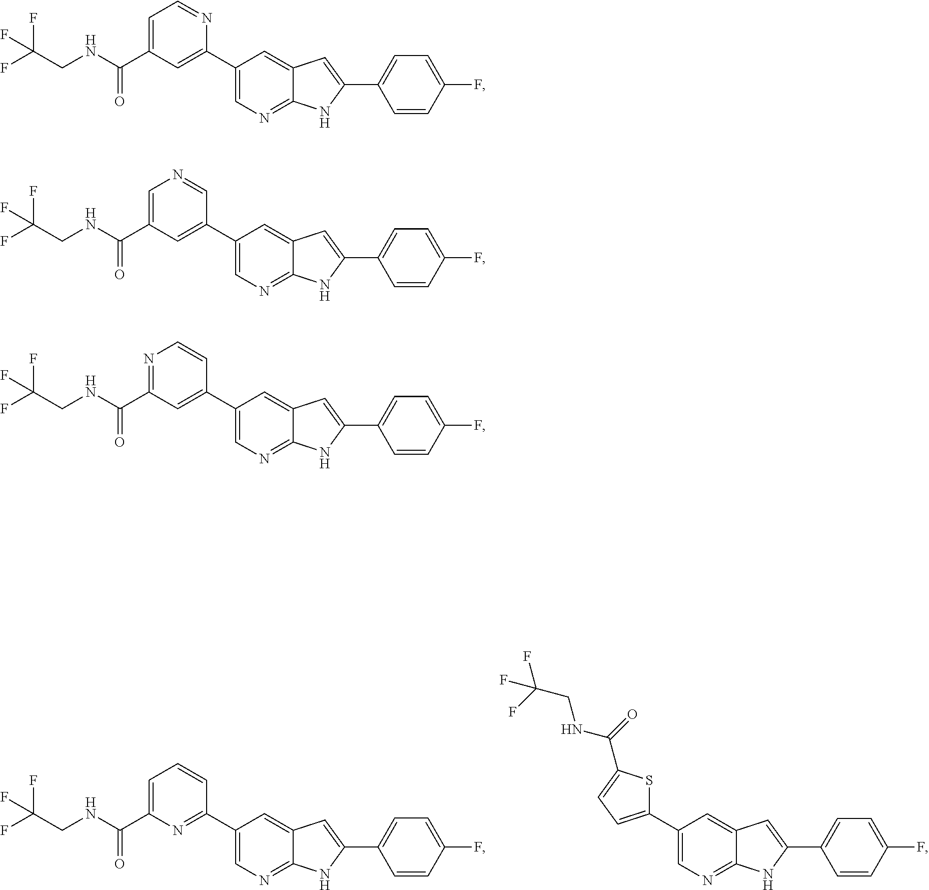 [SMILES: O=C(NCC(F)(F)F)c1ccnc(-c2cnc3[nH]c(-c4ccc(F)cc4)cc3c2)c1 ; O=C(NCC(F)(F)F)c1cncc(-c2cnc3[nH]c(-c4ccc(F)cc4)cc3c2)c1 ; O=C(NCC(F)(F)F)c1cc(-c2cnc3[nH]c(-c4ccc(F)cc4)cc3c2)ccn1 ; O=C(NCC(F)(F)F)c1cccc(-c2cnc3[nH]c(-c4ccc(F)cc4)cc3c2)n1 ; O=C(NCC(F)(F)F)c1ccc(-c2cnc3[nH]c(-c4ccc(F)cc4)cc3c2)s1] 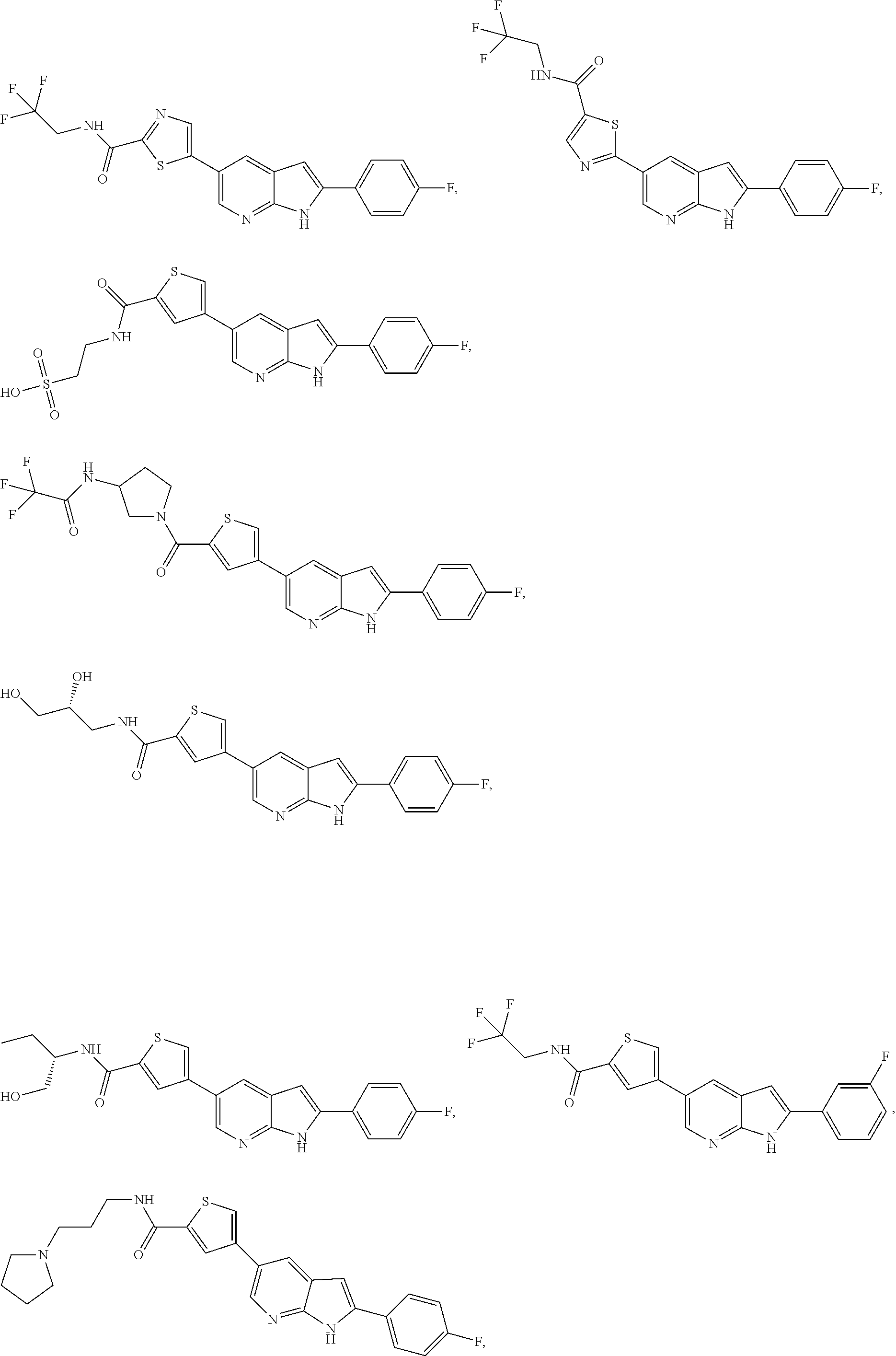 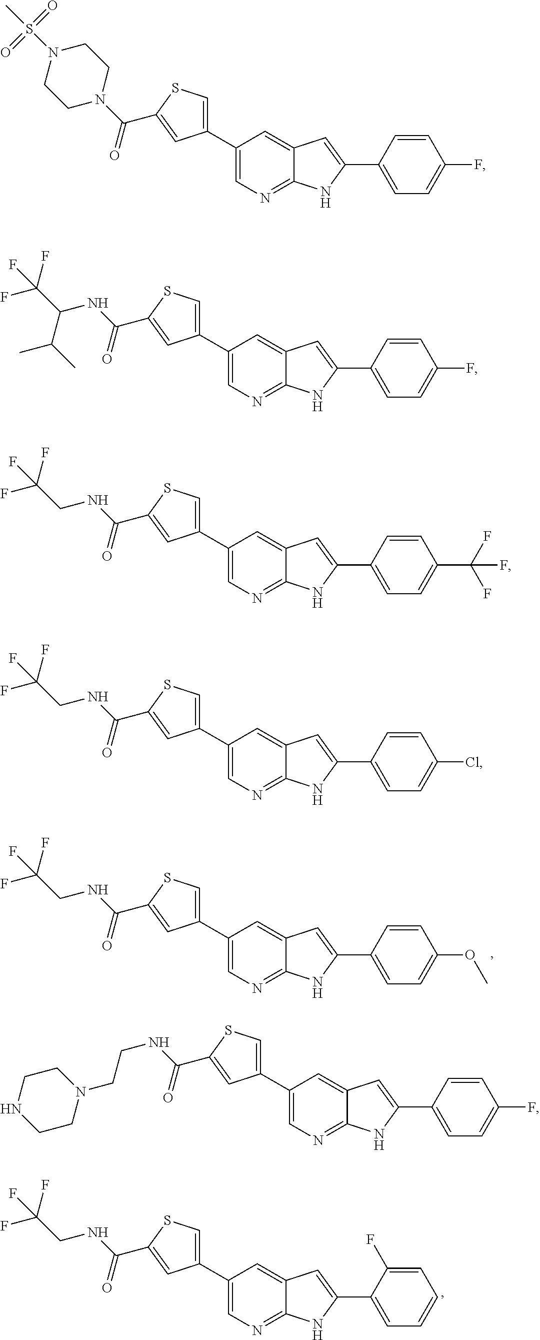 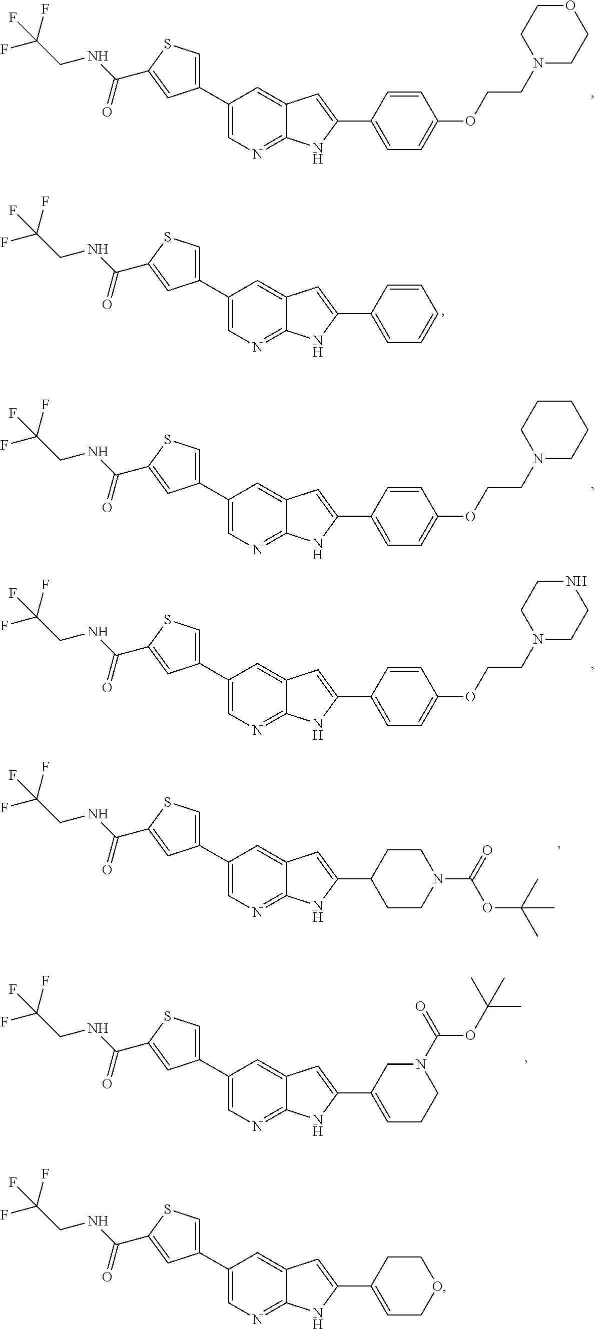 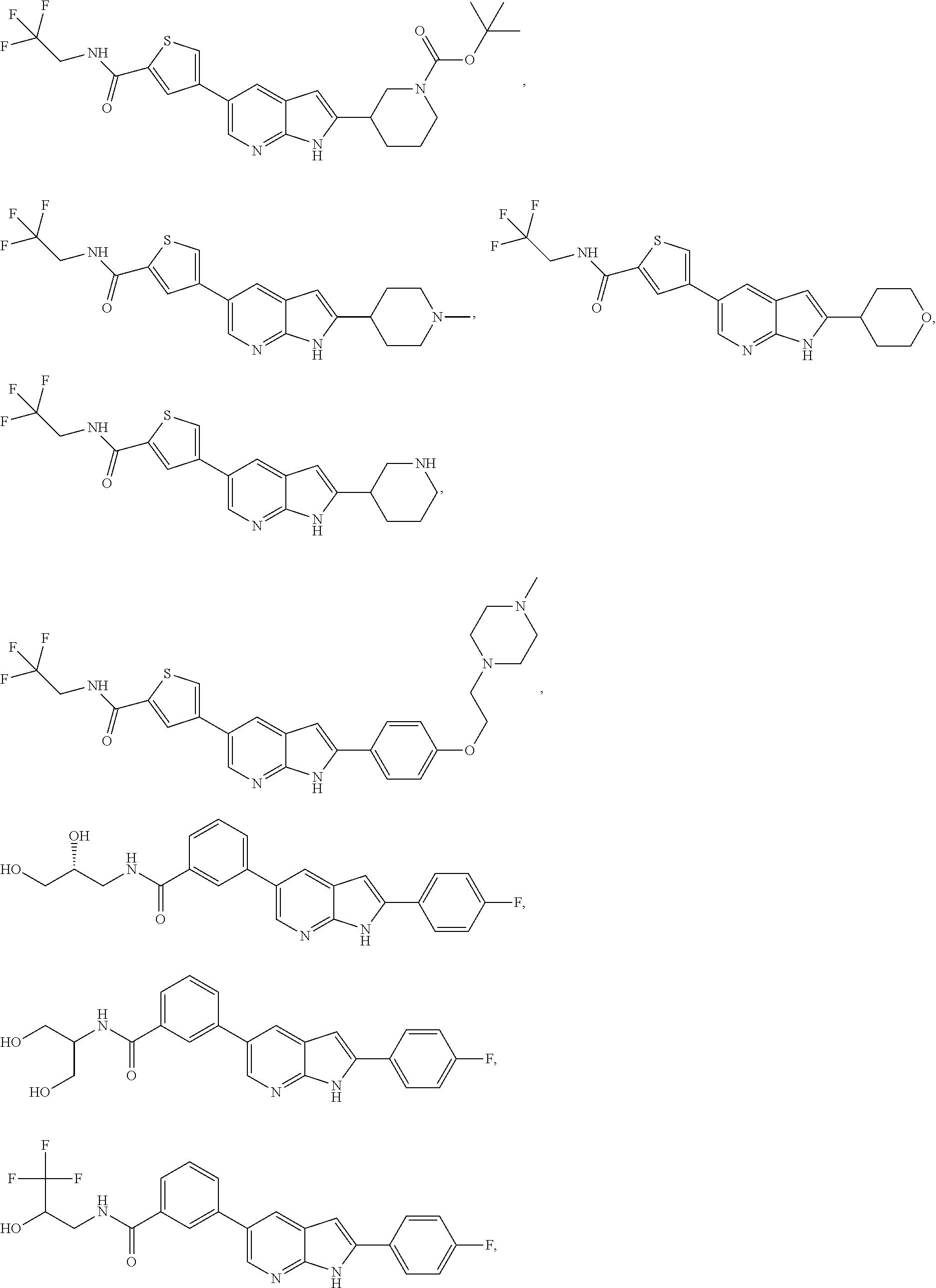 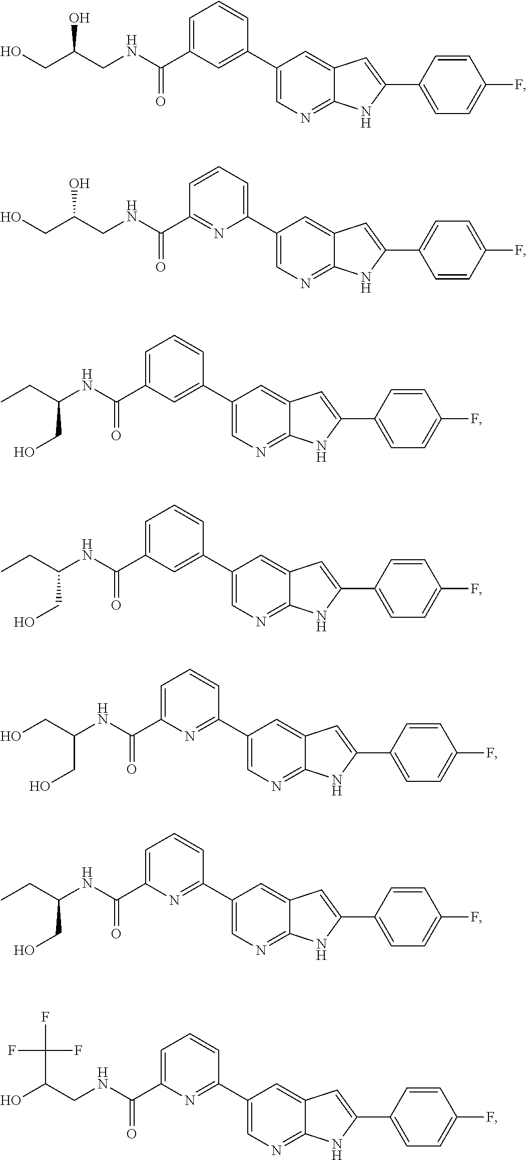 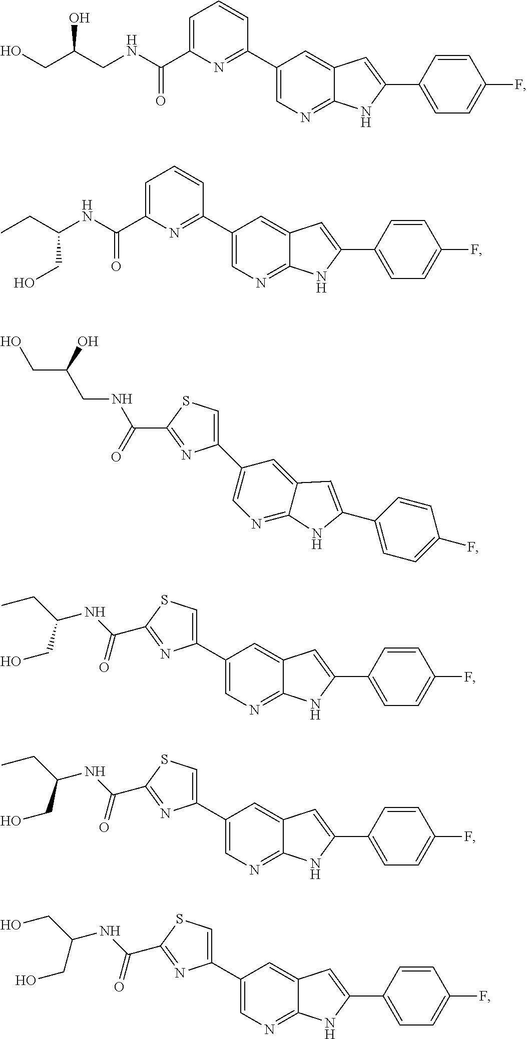 [SMILES: O=C(NC[C@H](O)CO)c1cccc(-c2cnc3[nH]c(-c4ccc(F)cc4)cc3c2)n1 ; CC[C@@H](CO)NC(=O)c1cccc(-c2cnc3[nH]c(-c4ccc(F)cc4)cc3c2)n1 ; O=C(NC[C@H](O)CO)c1nc(-c2cnc3[nH]c(-c4ccc(F)cc4)cc3c2)cs1 ; CC[C@@H](CO)NC(=O)c1nc(-c2cnc3[nH]c(-c4ccc(F)cc4)cc3c2)cs1 ; CC[C@H](CO)NC(=O)c1nc(-c2cnc3[nH]c(-c4ccc(F)cc4)cc3c2)cs1 ; O=C(NC(CO)CO)c1nc(-c2cnc3[nH]c(-c4ccc(F)cc4)cc3c2)cs1] 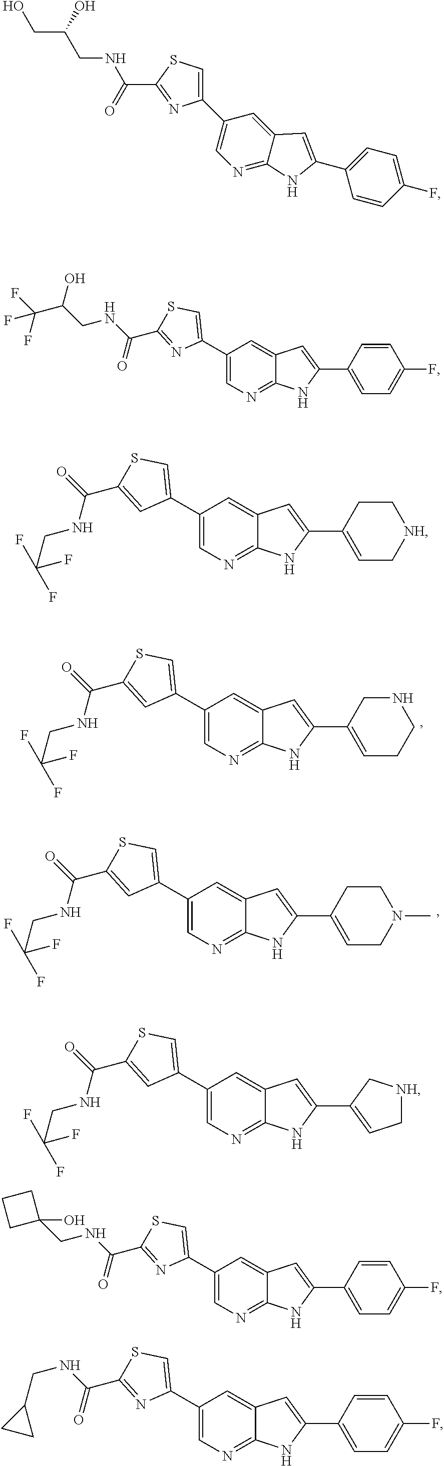 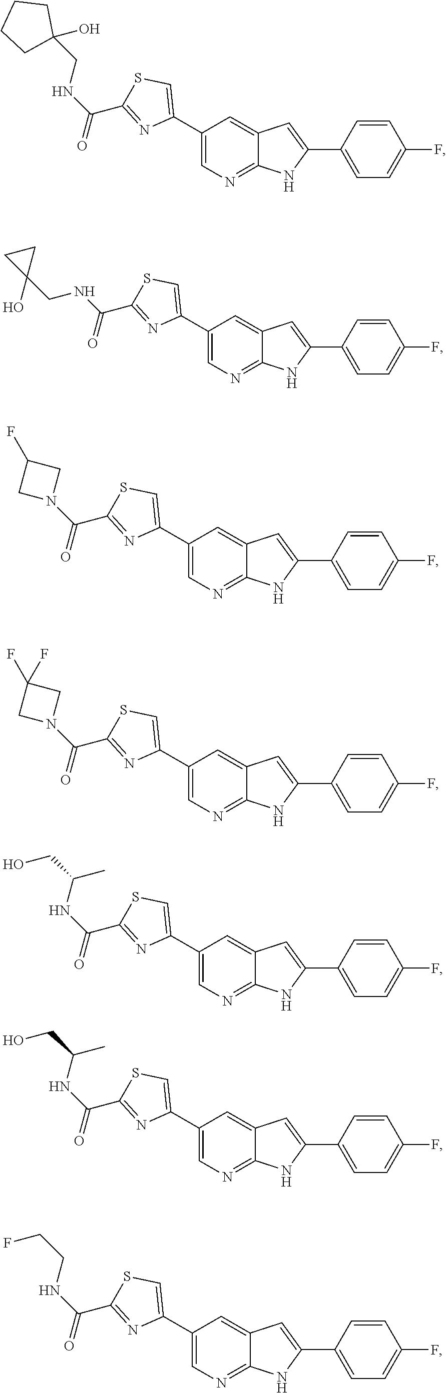 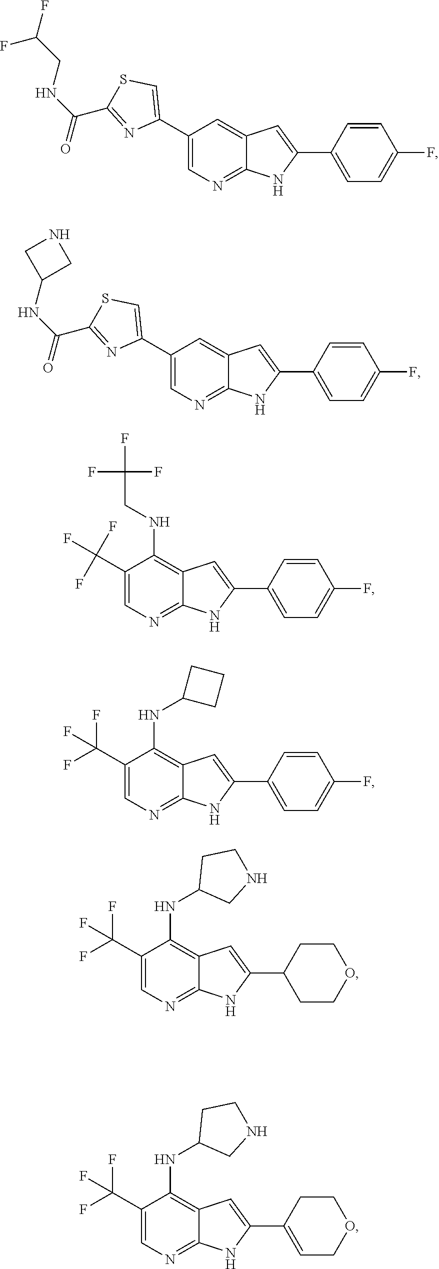 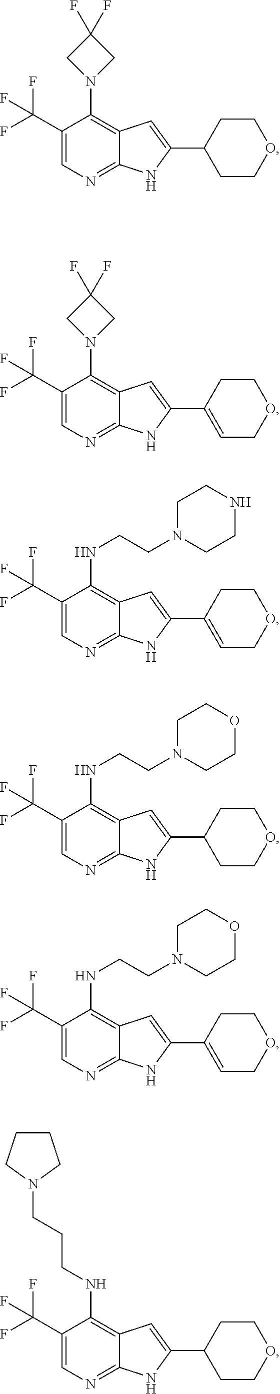 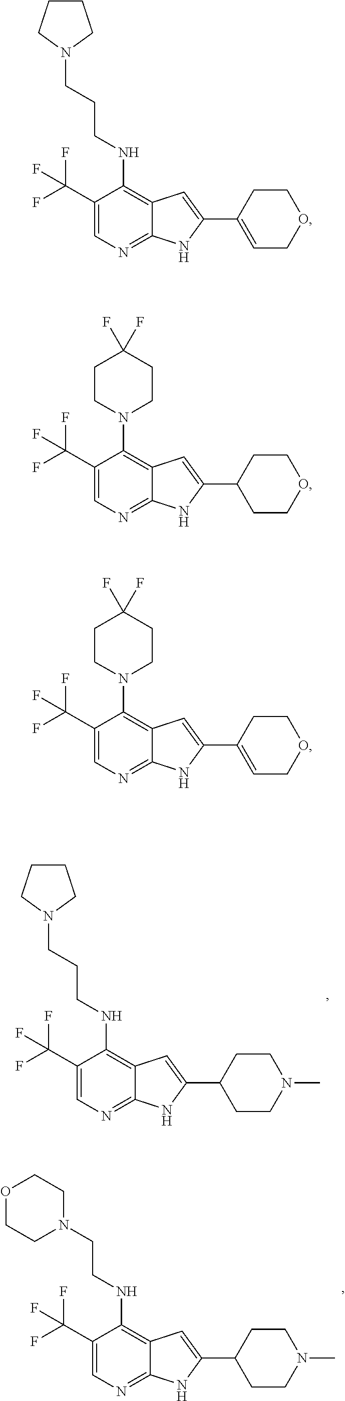 [SMILES: FC(F)(F)c1cnc2[nH]c(C3=CCOCC3)cc2c1NCCCN1CCCC1 ; FC1(F)CCN(c2c(C(F)(F)F)cnc3[nH]c(C4CCOCC4)cc23)CC1 ; FC1(F)CCN(c2c(C(F)(F)F)cnc3[nH]c(C4=CCOCC4)cc23)CC1 ; CN1CCC(c2cc3c(NCCCN4CCCC4)c(C(F)(F)F)cnc3[nH]2)CC1 ; CN1CCC(c2cc3c(NCCN4CCOCC4)c(C(F)(F)F)cnc3[nH]2)CC1] 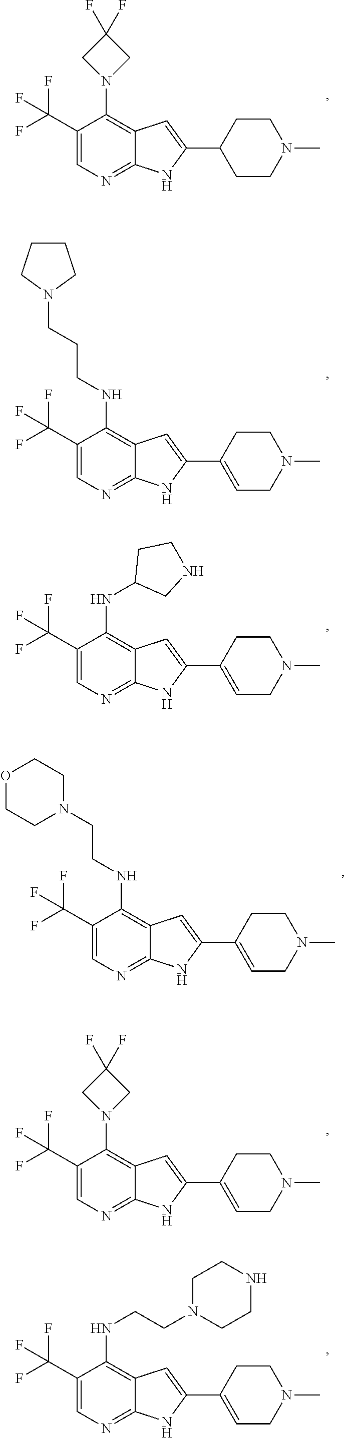 [SMILES: CN1CCC(c2cc3c(N4CC(F)(F)C4)c(C(F)(F)F)cnc3[nH]2)CC1 ; CN1CC=C(c2cc3c(NCCCN4CCCC4)c(C(F)(F)F)cnc3[nH]2)CC1 ; CN1CC=C(c2cc3c(NC4CCNC4)c(C(F)(F)F)cnc3[nH]2)CC1 ; CN1CC=C(c2cc3c(NCCN4CCOCC4)c(C(F)(F)F)cnc3[nH]2)CC1 ; CN1CC=C(c2cc3c(N4CC(F)(F)C4)c(C(F)(F)F)cnc3[nH]2)CC1 ; CN1CC=C(c2cc3c(NCCN4CCNCC4)c(C(F)(F)F)cnc3[nH]2)CC1] 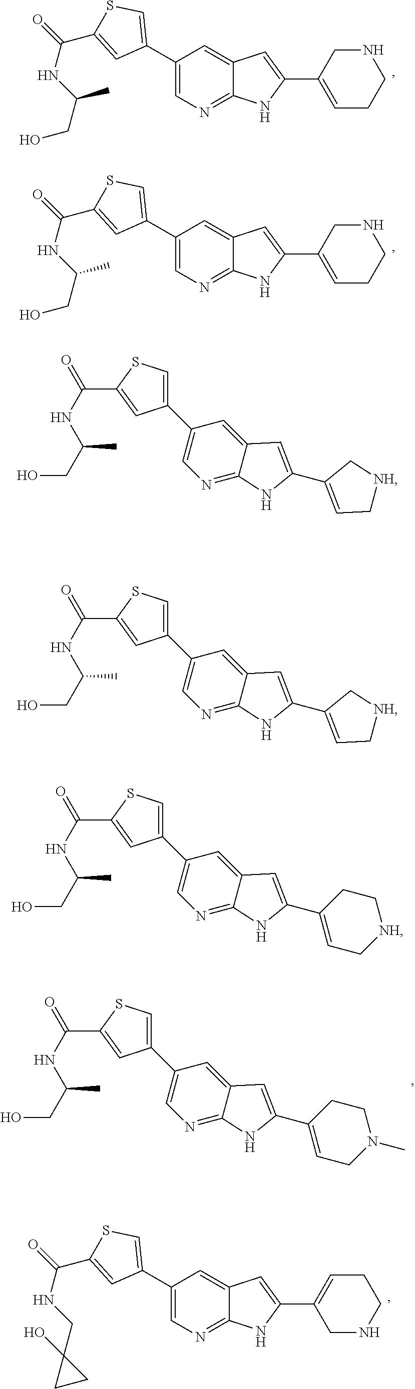 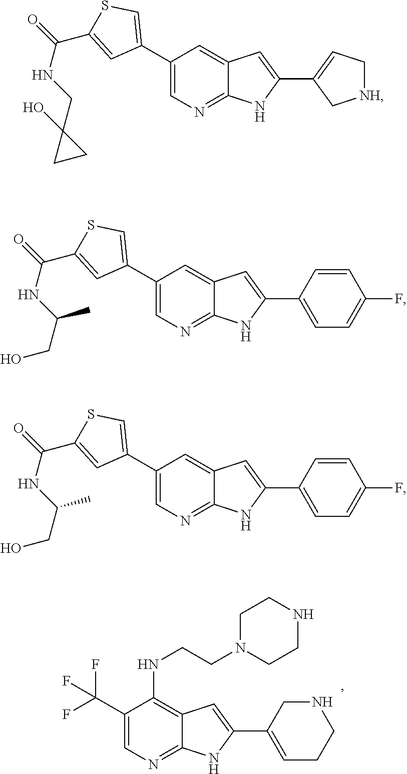 [SMILES: O=C(NCC1(O)CC1)c1cc(-c2cnc3[nH]c(C4=CCNC4)cc3c2)cs1 ; C[C@@H](CO)NC(=O)c1cc(-c2cnc3[nH]c(-c4ccc(F)cc4)cc3c2)cs1 ; C[C@H](CO)NC(=O)c1cc(-c2cnc3[nH]c(-c4ccc(F)cc4)cc3c2)cs1 ; FC(F)(F)c1cnc2[nH]c(C3=CCCNC3)cc2c1NCCN1CCNCC1] 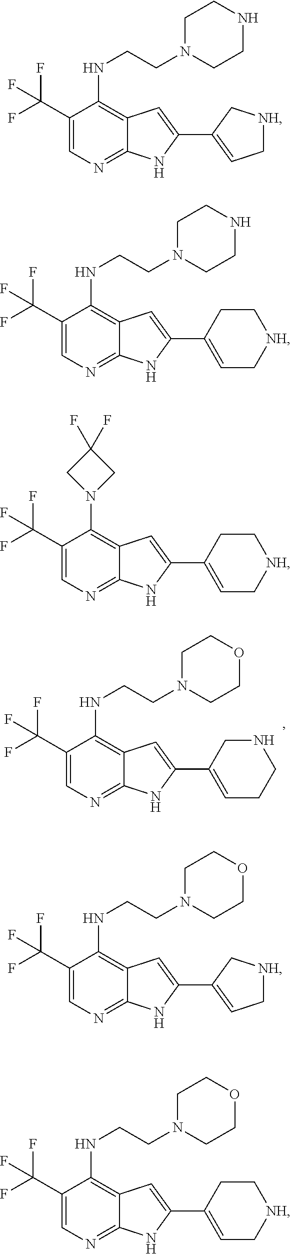 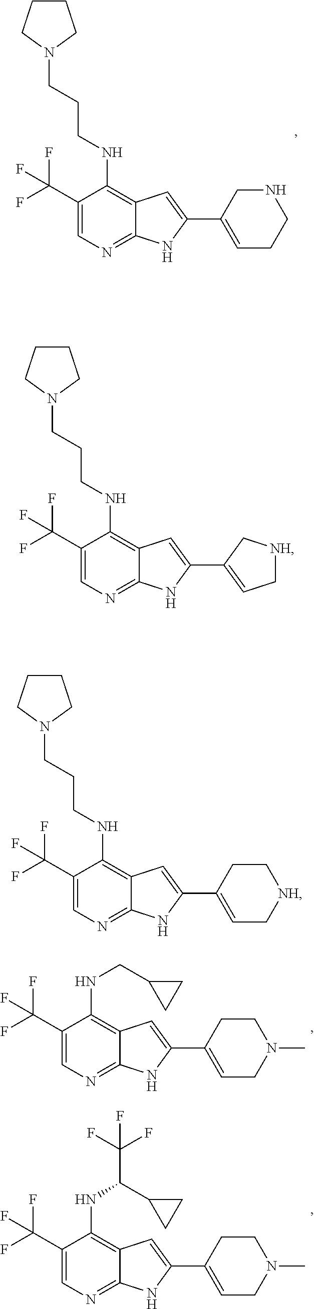 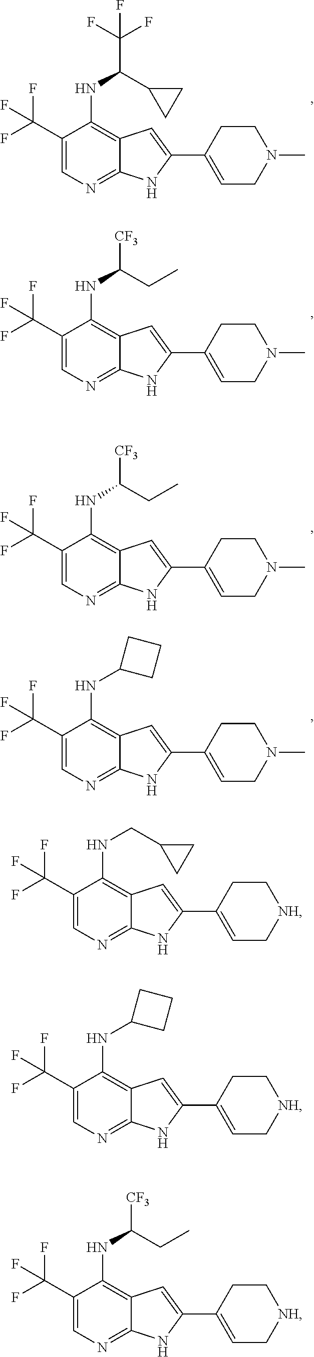 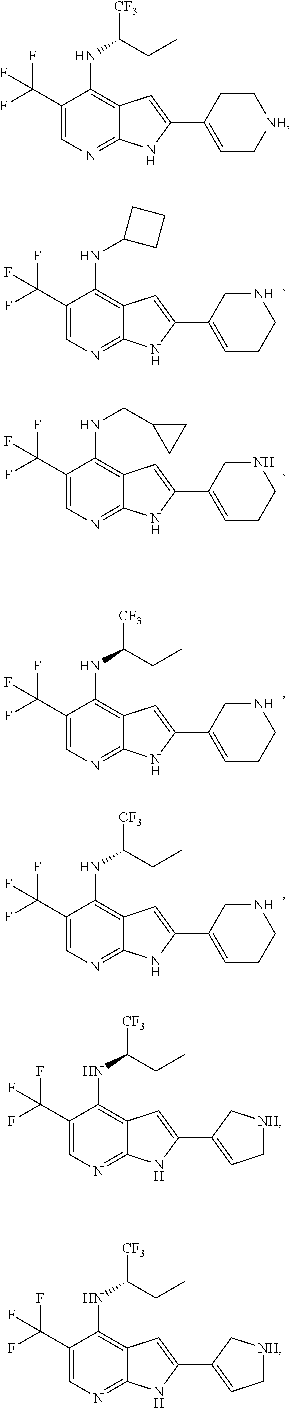 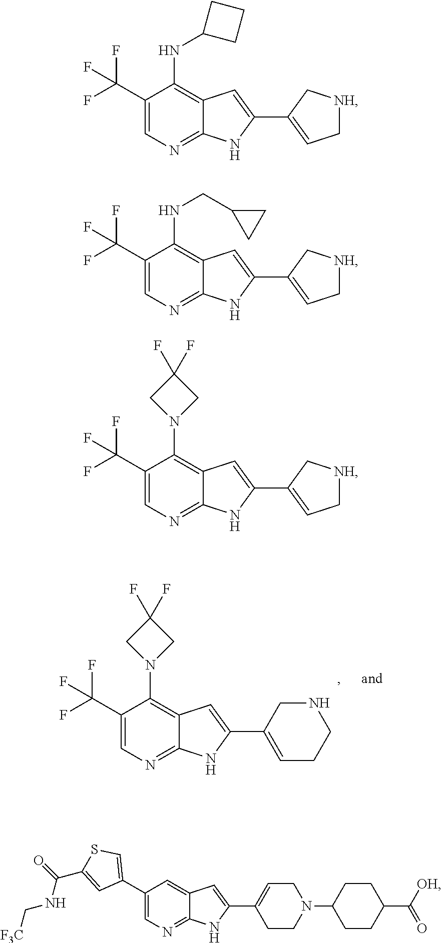
or a pharmaceutically acceptable salt, solvate, prodrug, or derivative thereof.

30. A pharmaceutical composition comprising a compound of claim 1, or a pharmaceutically acceptable salt, solvate, prodrug, or derivative thereof, and a pharmaceutically acceptable carrier or excipient.

31. A method of treating a disorder associated with abnormal cellular proliferation in a subject in need thereof comprising administering to the subject a therapeutically effective amount of a compound of claim 1, or a pharmaceutically acceptable salt, solvate, prodrug, or derivative thereof.

32. The method of claim 31, wherein the disorder comprises a cancer selected from lung cancer, pancreatic cancer, colorectal cancer, leukemias, lymphoma, melanoma, multiple myeloma, Ewing's sarcoma, osteosarcoma, gastric adenocarcinoma, cervical adenocarcinoma, and head and neck squamous cell carcinoma.

33. (canceled)
